SEQUENCE LISTING (13 sequences)

SEQ ID NO: 1 - DNA, 2037 bp, Homo sapiens
  SLC25A12
atggcggtcaaggtgcagacaactaagcgaggggatcctcatgagttaagaaacatatttctacagtatgccagtactgaggttgatggagagcgttatatgaccccagaagactttgttcagcgctatcttggactgtataatgatccaaatagtaacccaaagatcgtgcagctcttggcaggagtagctgatcaaaccaaggatgggttgatctcctatcaagagtttttggcatttgaatctgttttatgtgctccagattccatgttcatagtggctttccagttgtttgacaagagtggaaatggagaggtgacatttgaaaatgtcaaagaaatttttggacagactattattcatcatcatatcccttttaactgggattgtgaatttatccgactgcattttgggcataaccggaagaagcatcttaactacacagaattcacgcagtttctccaggagctgcaattggaacatgcaagacaagcctttgcactcaaagacaaaagcaaaagtggcatgatttctggtctggatttcagtgacatcatggttaccattagatctcacatgcttactccttttgtggaggagaacttagtttcagcagctggaggaagtatctcacaccaggttagcttctcctacttcaatgcatttaactcgttactgaataacatggagcttgttcgtaagatatatagcactctagctggcacaaggaaagatgttgaagtcacaaaggaggaatttgcccagagtgccatacgctatggacaagtcacaccactagaaattgatattctatatcagcttgcagacttatataatgcttcagggcgcttgactttggcagatattgagagaatagccccattggctgagggggccttaccttacaacctggcagaacttcagagacagcagtctcctgggttaggcaggcctatctggctccagattgccgagtctgcttacagattcactctgggctcagttgctggagctgtgggagccactgcagtgtatcctatagatctggtgaagacccgaatgcaaaaccagcgtggctctggctctgttgttggggagctaatgtacaaaaacagctttgactgttttaagaaagtcttgcgttatgagggcttctttggactctacaggggtctgataccacaacttataggggttgctccagaaaaggccattaaactgactgttaatgattttgttcgggacaaatttaccagaagagatggctctgttccacttccagcagaagttcttgctggaggctgtgctggaggctctcaggtcatttttaccaacccattggagatagtgaagattcgtctgcaagtagctggagagatcaccacgggacccagagtcagcgccctgaatgtgctccgggacttgggaatttttggtctgtataagggtgccaaagcgtgtttcctccgagacattcccttctctgcaatctattttcctgtttatgctcattgcaaactacttctggctgatgaaaatggacacgtgggaggtttaaatcttcttgcagctggagccatggcaggtgtcccagctgcatctctggtgacccctgctgatgtcatcaagacaagactgcaggtggctgcccgcgctggccagacgacatacagtggtgtcatcgactgtttcaggaagattctccgggaagaagggccctcagcattttggaaagggactgcagctcgagtgtttcgatcctctccccagtttggtgttaccttggtcacttatgaacttctccagcggtggttttacattgattttggaggcctcaaacccgctggttcagaaccaacacctaagtcacgcattgcagaccttcctcctgccaaccctgatcacatcggtggatacagactcgccacagccacgtttgcaggcatcgaaaacaaatttggcctttatctcccgaaatttaagtctcctagtgttgctgtggttcagccaaaggcagcagtggcagccactcagtga

SEQ ID NO: 2 - protein, 678 aa, Homo sapiens
  SLC25A12
MAVKVQTTKRGDPHELRNIFLQYASTEVDGERYMTPEDFVQRYLGLYNDPNSNPKIVQLLAGVADQTKDGLISYQEFLAFESVLCAPDSMFIVAFQLFDKSGNGEVTFENVKEIFGQTIIHHHIPFNWDCEFIRLHFGHNRKKHLNYTEFTQFLQELQLEHARQAFALKDKSKSGMISGLDFSDIMVTIRSHMLTPFVEENLVSAAGGSISHQVSFSYFNAFNSLLNNMELVRKIYSTLAGTRKDVEVTKEEFAQSAIRYGQVTPLEIDILYQLADLYNASGRLTLADIERIAPLAEGALPYNLAELQRQQSPGLGRPIWLQIAESAYRFTLGSVAGAVGATAVYPIDLVKTRMQNQRGSGSVVGELMYKNSFDCFKKVLRYEGFFGLYRGLIPQLIGVAPEKAIKLTVNDFVRDKFTRRDGSVPLPAEVLAGGCAGGSQVIFTNPLEIVKIRLQVAGEITTGPRVSALNVLRDLGIFGLYKGAKACFLRDIPFSAIYFPVYAHCKLLLADENGHVGGLNLLAAGAMAGVPAASLVTPADVIKTRLQVAARAGQTTYSGVIDCFRKILREEGPSAFWKGTAARVFRSSPQFGVTLVTYELLQRWFYIDFGGLKPAGSEPTPKSRIADLPPANPDHIGGYRLATATFAGIENKFGLYLPKFKSPSVAVVQPKAAVAATQ

SEQ ID NO: 3 - DNA, 2031 bp, Homo sapiens
  SLC25A13
atggcggccgccaaggtggctttaaccaagagagcagatccagctgagcttagaacaatatttttgaagtatgcaagcattgagaaaaacggtgaatttttcatgtcccccaatgactttgtcactcgatacttgaacatttttggagaaagccagcctaatccaaagactgtggaacttttaagtggagtggtggatcagaccaaagatggattaatatcttttcaagaatttgttgcctttgaatctgtcctgtgtgcccctgatgctttgtttatggtagcctttcagctgtttgacaaagctggcaaaggagaagtaacttttgaggatgttaagcaagtttttggacagaccacaattcatcaacatattccatttaactgggattcagaatttgtgcaactacattttggaaaagaaagaaaaagacacctgacatatgcggaatttactcagtttttattggaaatacaactggagcacgcaaagcaagcctttgtgcaacgggacaatgctaggactgggagagtcacagccatcgacttccgagacatcatggtcaccatccgcccccatgtcttgactccttttgtagaagaatgtctagtagctgctgctggaggtaccacatcccatcaagttagtttctcctattttaatggatttaattcgctccttaacaacatggaactcattagaaagatctatagcactctggctggcaccaggaaagatgttgaagtgactaaggaggagtttgttctggcagctcagaaatttggtcaggttacacccatggaagttgacatcttgtttcagttagcagatttatatgagccaaggggacgtatgaccttagcagacattgaacggattgctcctctggaagagggaactctgccctttaacttggctgaggcccagaggcagcagaaggcctcaggtgattcagctcgaccagttcttctacaagttgcagagtcggcctacaggtttggtctgggttctgttgctggagctgttggagccactgctgtgtatcctatcgatcttgtaaaaactcgaatgcagaaccaacgatcaactggctcttttgtgggagaactcatgtataaaaacagctttgactgttttaagaaagtgctacgctatgaaggcttctttggactgtatagaggtctgttgccacagttattgggagttgccccagagaaggccataaaacttacagtgaacgattttgtgagggataaatttatgcacaaagatggttcggtcccacttgcagcagaaattcttgctggaggctgcgctggaggctcccaggtgattttcacaaatcctttagaaatcgtcaagatccgtttgcaagtggcaggagaaatcaccactggtcctcgagtcagtgctctgtctgtcgtgcgggacctggggttttttgggatctacaagggtgccaaagcatgctttctgcgggacattcctttctcggccatctactttccgtgctatgctcatgtgaaggcttcctttgcaaatgaagatgggcaggttagcccaggaagcctgctcttagctggtgccatagctggtatgcctgcagcatctttagtgacccctgctgatgttatcaagacgagattacaggtggctgcccgggctggccaaaccacttacagcggagtgatagactgctttagaaagatactgcgtgaagaaggaccaaaagctctgtggaagggagctggtgctcgtgtatttcgatcctcaccccagtttggtgtaactttgctgacttacgaattgctacagcgatggttctacattgattttggaggagtaaaacccatgggatcagagccagttcctaaatccaggatcaacctgcctgccccgaatcctgatcacgttgggggctacaaactggcagttgctacatttgcagggattgaaaacaaatttggactttacctacctctcttcaagccatcagtatctacctcaaaggctattggtggaggcccatag

SEQ ID NO: 4 - protein, 676 aa, Homo sapiens
  SLC25A13
MAAAKVALTKRADPAELRTIFLKYASIEKNGEFFMSPNDFVTRYLNIFGESQPNPKTVELLSGVVDQTKDGLISFQEFVAFESVLCAPDALFMVAFQLFDKAGKGEVTFEDVKQVFGQTTIHQHIPFNWDSEFVQLHFGKERKRHLTYAEFTQFLLEIQLEHAKQAFVQRDNARTGRVTAIDFRDIMVTIRPHVLTPFVEECLVAAAGGTTSHQVSFSYFNGFNSLLNNMELIRKIYSTLAGTRKDVEVTKEEFVLAAQKFGQVTPMEVDILFQLADLYEPRGRMTLADIERIAPLEEGTLPFNLAEAQRQQKASGDSARPVLLQVAESAYRFGLGSVAGAVGATAVYPIDLVKTRMQNQRSTGSFVGELMYKNSFDCFKKVLRYEGFFGLYRGLLPQLLGVAPEKAIKLTVNDFVRDKFMHKDGSVPLAAEILAGGCAGGSQVIFTNPLEIVKIRLQVAGEITTGPRVSALSVVRDLGFFGIYKGAKACFLRDIPFSAIYFPCYAHVKASFANEDGQVSPGSLLLAGAIAGMPAASLVTPADVIKTRLQVAARAGQTTYSGVIDCFRKILREEGPKALWKGAGARVFRSSPQFGVTLLTYELLQRWFYIDFGGVKPMGSEPVPKSRINLPAPNPDHVGGYKLAVATFAGIENKFGLYLPLFKPSVSTSKAIGGGP

SEQ ID NO: 5 - DNA, 3876 bp, Drosophila <fruit fly, genus>
  CYFIP / Sra-1
atgacggagaagattacgctagccgacgcgctgtccaacgtggaggtgttggacgagctatccctgccggacgagcagccctgcatcgaggcgcagccctgctcgattatctacaaggcaaacttcgatacgaatttcgaggatcgcaatggatttgtcacgggtatcgccaagtacatcgaggaggccaccacccatgccaacttgaatgtgctcctggacgaggggcagaaacacgcagtaatgctctacacctggcgctgctgctcgcgcgccattccgcagcccaagtccaatgagcagccgaatcgcgtggagatctacgagaagacggtcgaggtgctagccccggaggtgaacaagctgctcaacttcatgtacttccaacgcaaggccatcgaggccttctccggcgaggtgaagcgcttgtgccacgccgagaagcgcaaggacttcgtgtccgaggcgtacctattgacgctaggcaagtttatcaacatgtttgccgtgctggacgagctgaagaacatgaagtccagcgtgaagaacgattactccacctacaggcgagctgcgcagttcctcaaagtgatgtcggactcgcacaccttgcaggagtcgcagaatctgtccatgttcctggccacgcagaacaagattcgcgacacggtcaaggacacgctggagaagatcgtgggctacgaggatctgctctcagatgtggtcaacatttgcgtgcatatgttcgagacaaagatgtacttgacccctgaggagaagcacatgctggtaaaagttatgggctttggcctgttccttatggacagcgatgcgtgcaatatcaacaaacttgatcaaaagaagaagatacggctggatcgcatcgatcgcatctttaagaacctggaagtggtgcccctcttcggcgacatgcagattgctcccttcaactacataaagcgcagcaagcacttcgattccagcaagtggccgctgtccagctcaaacgccatcagcccacaggcggatctaatggtgcatttgccgcagatacgcgaggatcacgtcaagtacatttcggagctggcgcgatacacaaacgaggtgaccaccaccgtcaaggagaatccctctgatgccgagaaccggattacggccgacctggctctgcgcggccttcaattgctgtccgagtggaccagtgtggtcaccgagttgtactcctggaagctcctgcatccgactgatcatcaccagaacaaggagtgccccgtggaggctgaggagtacgagcgggccacccgctacaattacacctcggaagagaagttcgcgctgatcgaggtcatcgccatgataaagggactgcaggtgctaatggcgcgcattgaaaccgtattgtgcgaggctatccggcgtaacatctattccgagctgcaggacttcgtgcagctttcgcttcgtgaaccgctgcgaaaggccgtaaaaaataagaaggatcttatccggagcatcatcatgtcggtgcgagagacatcggcggactggcaaaagggctatgagcccaccgacgatccagtggccaagggcaagaaggatcccgacggcggcttccgcatccaggtgcctcgcctcaatgtgggaccttcctccactcagctatacatggtgcgcaccatgctggagtctctgattgccgacaaaagcggcggcaagcgcactttgcgcaaggacatcgacggaaactgtttgctgcagatcgacacgttccacaagacatcgttctactggagctacttgctcaacttcagcgacacgcttcagaagtgctgcgatctctcgcagctgtggtatcgcgagttctacttggagatgaccatgggtcgtaaggttaacaagtgcttggtgcggcatcagcacaacgaggagtgcaaggatctgatcaccatggagaagcgcattcagtttccgattgaaatgtccatgccctggatcctaaccgatcacattctgcaaacgaaggagccctcaatgatggaatttgtcttgtatcccctggacttgtacaacgactcggcgtactacgcactcactgtcttccgaaagcagttcctgtacgacgaagtggaggccgaagtcaatctgtgcttcgatcagtttgtctacaagctgagcgagcagatctttgcccattacaagcaattggcgggcagcatcttcttggacaagcgattccgtttggagtgcgaggtactcggtttcaacttccagtcgtatccccgcaataatcgttatgagaccttgttaaaacaacgacatgtccaacttttgggccgttctattgacttgaacaaactgatcacacaacgcataaacgccaacatgcacaagagcattgagttggccatcagtcgtttcgagggcaacgatataaccggcattgtggaactcgaaggcctgctagaggcgaatcgaatttgccacaagttgctaagcaagtacttggccctggataactttgacggcatggtaaaggaggctaaccacaatgtactggcgccttacggcagaataacactgcacgtctttgtggaattgaactacgactttctggtcaattactgctataatgcagccacgaatcgtttcattcgaaccaaagtgaatctttcgtcgtcacaggctattcagcgtgagaagccgccacaaatgtcccattactatctgtggggttccaagcagctgaatgctgcctattccacacagtatggccagtacactggctttgtaggttcaccgcatttccatgccatgtgccgcctgctgggctatcagggcatagcagtcgtcatggacattatactgaaggacattgttaagccactgatccagggctcgctgctgcagttcaccaagacgctgatgattgccatgcccaagtcatgcaaactaccccgttgcgaatatggttcgcctggagtgctcagctactatcaggctcatcttacagacatcgtgcaatatccggacgcgaagacagagctatttcagtctttccgggagtttggcaacagcattatcttttgcctgctgatcgaacaggcgctctcgcaggaggaggtgtgcgatctactgcacgccgccctcttccagaacatctttcccagacctttttgcaaagagaatgaaaaaccggaggctaagcaaaagcgtcttgaagcacagtttgccaatctgcagatagtttcaaacgtggaaaagattggcactgcaaagcaagcaatgatagcacgagagggagatttgctgactcgtgagcgtctttgctgtggcctcagcatttttgaagttattctcaaccgcgtgaagagctatttggacgaccctgtgtggtgtggtcctccaccggccaacgggattatccacgtggatgagtgctcggagttccatcgcctttggtcggctctgcagttcgtttattgcattccagtcaggggaacggagtacaccatcgaggagctcttcggcgaaggtctgaactgggctggctgcgtcatgatcgtactgctgggacagcagcgtcgcttcgaggctctggacttttgctaccacattctgcgggtacagcgcgtcgatggcaaggatgaggacgtaaagggcattcaattgaagcgaatggtggatcggattcgtcggttccaagtattaaactcacaaatcttctccattctcaacaagtatctaaagggcggcgatggcgagggctcaaatgtggagcacgtgcggtgttttccaccgcctcagcatccctcggtcatatcgtcctcgtcgcactaccaggatccccagaagctgcggcagagcataaacaattga

SEQ ID NO: 6 - RNA, 21 nt, Artificial Sequence
  Aralar primer
tcctgggactcttttccgaat

SEQ ID NO: 7 - RNA, 19 nt, Artificial Sequence
  Aralar primer Rev
gcctggaactccgagaagg

SEQ ID NO: 8 - RNA, 20 nt, Artificial Sequence
  FLuc Primer
atctgcctcctggcttcaac

SEQ ID NO: 9 - RNA, 20 nt, Artificial Sequence
  FLuc Primer Rev
cggtagacccagagctgttc

SEQ ID NO: 10 - protein, 393 aa, Artificial Sequence
  Aralar1 CTD
LADIERIAPLAEGALPYNLAELQRQQSPGLGRPIWLQIAESAYRFTLGSVAGAVGATAVYPIDLVKTRMQNQRGSGSVVGELMYKNSFDCFKKVLRYEGFFGLYRGLIPQLIGVAPEKAIKLTVNDFVRDKFTRRDGSVPLPAEVLAGGCAGGSQVIFTNPLEIVKIRLQVAGEITTGPRVSALNVLRDLGIFGLYKGAKACFLRDIPFSAIYFPVYAHCKLLLADENGHVGGLNLLAAGAMAGVPAASLVTPADVIKTRLQVAARAGQTTYSGVIDCFRKILREEGPSAFWKGTAARVFRSSPQFGVTLVTYELLQRWFYIDFGGLKPAGSEPTPKSRIADLPPANPDHIGGYRLATATFAGIENKFGLYLPKFKSPSVAVVQPKAAVAATQ

SEQ ID NO: 11 - protein, 391 aa, Artificial Sequence
  Citrin CTD
TLADIERIAPLEEGTLPFNLAEAQRQQKASGDSARPVLLQVAESAYRFGLGSVAGAVGATAVYPIDLVKTRMQNQRSTGSFVGELMYKNSFDCFKKVLRYEGFFGLYRGLLPQLLGVAPEKAIKLTVNDFVRDKFMHKDGSVPLAAEILAGGCAGGSQVIFTNPLEIVKIRLQVAGEITTGPRVSALSVVRDLGFFGIYKGAKACFLRDIPFSAIYFPCYAHVKASFANEDGQVSPGSLLLAGAIAGMPAASLVTPADVIKTRLQVAARAGQTTYSGVIDCFRKILREEGPKALWKGAGARVFRSSPQFGVTLLTYELLQRWFYIDFGGVKPMGSEPVPKSRINLPAPNPDHVGGYKLAVATFAGIENKFGLYLPLFKPSVSTSKAIGGGP

SEQ ID NO: 12 - DNA, 3864 bp, Homo sapiens
  CYFIP1, coding sequence
atggggccgcaggggccggccgggggggtccccagcgggctctgcgcgccgacctgcagctgggacccgcgcctggaaccctcccgacgagcgcagcccaggatggcggcccaggtgactctggaggacgcgctgtccaacgtggacctcctggaggagctgcccctgcccgaccagcagccctgcatcgagcccccgccatcctcgctgctctaccagccaaatttcaacactaactttgaagacagaaatgcatttgttactggcatcgcaagatacattgaacaagccaccgtccactctagcatgaacgagatgctggaggagggccaagaatatgctgtcatgctgtacacctggaggagctgctcccgggccatcccacaggtgaaatgtaacgagcagcctaacagagtggaaatctacgagaaaaccgtggaggttctggagcctgaggtcacaaaactgatgaatttcatgtacttccagagaaatgccattgagcgtttctgcggggaagtgaggcgcctgtgccatgccgagaggaggaaggacttcgtgtcagaagcctacctgatcacactgggcaaattcatcaacatgttcgctgtgctggacgagctgaagaacatgaagtgcagtgtgaagaacgaccactcagcgtacaagagggccgctcagtttttacgtaaaatggcagatccacagtccatccaggaatcgcagaatctgtccatgttcctggccaatcataacaagatcacacagtctctgcagcagcagctcgaagtgatttctggctacgaagagctcctggcagatattgtgaatctgtgtgtggattactacgagaacaggatgtatttgacgcccagtgagaaacacatgcttctcaaagtcatgggatttggtctgtacctgatggatgggagtgtcagtaacatctataagttggatgccaagaaaagaataaacttatccaaaatcgacaagtacttcaagcaactccaggtggttccgctatttggggacatgcaaatagaactggcaagatatatcaagaccagcgcccactacgaggaaaataaatctcgatggacgtgcacatcctccggcagcagccctcagtacaacatctgcgagcagatgatccagatccgcgaggaccacatgcgcttcatttcggagctggcgcgctacagcaacagcgaggtggtcacgggctcgggccgccaggaggcccagaagacggacgcggagtaccgcaagctcttcgacctggcgctgcagggcctgcagctgttgtcgcagtggagcgcgcacgtgatggaagtgtattcctggaagcttgtgcaccccaccgacaagtactccaacaaggactgccccgacagcgctgaagagtacgagcgtgccacgcgctacaactacaccagcgaggagaagtttgccctagtggaggtgatcgccatgatcaaaggcctgcaggtgctgatgggcaggatggagagcgtgttcaaccacgccatccggcacaccgtctatgccgcactgcaggacttctcccaggtgacccttagggagccgctgcggcaggccatcaagaagaagaagaacgtcatccagagtgtcctgcaggccatcaggaagaccgtgtgtgactgggagacggggcatgagcccttcaatgacccagccttgcggggcgagaaggaccccaagagcggcttcgacataaaagtaccacgccgcgccgtgggaccctccagcactcagctttacatggtgagaaccatgctagagtccctcattgcagacaaaagtggttccaagaaaaccttgagaagtagccttgaggggcccaccatattggacatagaaaaatttcatcgagagtcattcttctacactcacttgataaatttcagtgaaacgctgcagcagtgctgtgacctttcgcagctgtggttccgagagttcttcctggagctgaccatgggcaggaggatccagttccccattgagatgtcgatgccctggatcctgacggaccacatcctggagaccaaggaggcatcgatgatggagtacgtgctctactccctggacctgtacaatgacagcgcccactacgcgctcaccaggttcaacaagcagttcctgtacgacgaaattgaggccgaggtgaatctatgttttgaccaatttgtttacaagctagcagaccagatatttgcctattataaggttatggcaggaagtttgcttcttgataaacggttacgatcagaatgcaagaatcagggagccacgatccacctcccgccgtctaaccgctacgagacgctgctgaagcagaggcatgtgcagctcctcggcagatcaatagacctcaatcgtctgatcacccagcgcgtctcagcagccatgtataagtccctagaactggcgattggacgatttgaaagtgaagatttgacctccatagttgagctggatggcctgttggaaatcaaccgcatgacccacaagctgctgagccggtacctgacgctggacggcttcgacgccatgttccgggaggccaaccacaacgtgtcagcgccctacgggaggatcaccctgcacgtcttctgggagctcaactatgacttcctgcccaactactgctacaacggctctaccaaccggtttgttcggacagtgttaccattttctcaggaatttcaaagagataagcagcctaatgcacagcctcagtatctgcatggatccaaggctttgaacttggcctactccagcatttacggcagctaccggaacttcgtgggacctccacactttcaagtcatctgccggcttctcggctaccagggtatcgccgtggtcatggaggagctgctgaaggtcgtcaagagcctgctgcaaggcacaatcctgcagtacgtgaagacgctgatggaggtgatgcccaagatctgccgcctgccccggcacgagtacggctctcctggtatcctggagttcttccaccaccagctgaaggacatcgtggagtacgcagagctgaagacggtgtgcttccagaacctgcgggaggtggggaacgccatcctcttctgcctgctcatcgagcagagcctgtctttagaagaagtgtgtgacctgctgcacgcggctcctttccagaacatcttgccgcgagtccatgtgaaagagggggagagacttgatgccaaaatgaaaagactagaatcaaagtacgccccgctgcatcttgtcccactgattgaaagactggggacccctcagcaaattgccatcgcaagagagggggacctgctgacaaaggagcgcctctgctgcggcctgtccatgtttgaggtcatcctgacacggatccggagctttctggatgaccccatctggcgcgggcctctgcccagcaatggggtcatgcatgtggacgagtgtgtggagtttcacagactgtggagtgccatgcagtttgtctactgcattcccgtggggacacacgagttcacagtcgagcagtgctttggtgatgggctacactgggctggctgtatgatcatcgtacttcttgggcagcagcggcgttttgctgtgctggatttctgctaccatctacttaaagtccagaaacatgatggcaaagatgagattattaaaaatgtgcctttgaagaagatggtggagagaattcgcaagttccagattctcaatgatgagatcatcaccatcctggataagtacctgaagtcaggcgacggggagggcacgccagtggagcatgtgcgctgcttccagccgcccatccaccagtccctcgccagcagctga

SEQ ID NO: 13 - protein, 1287 aa, Homo sapiens
  CYFIP1
MGPQGPAGGVPSGLCAPTCSWDPRLEPSRRAQPRMAAQVTLEDALSNVDLLEELPLPDQQPCIEPPPSSLLYQPNFNTNFEDRNAFVTGIARYIEQATVHSSMNEMLEEGQEYAVMLYTWRSCSRAIPQVKCNEQPNRVEIYEKTVEVLEPEVTKLMNFMYFQRNAIERFCGEVRRLCHAERRKDFVSEAYLITLGKFINMFAVLDELKNMKCSVKNDHSAYKRAAQFLRKMADPQSIQESQNLSMFLANHNKITQSLQQQLEVISGYEELLADIVNLCVDYYENRMYLTPSEKHMLLKVMGFGLYLMDGSVSNIYKLDAKKRINLSKIDKYFKQLQVVPLFGDMQIELARYIKTSAHYEENKSRWTCTSSGSSPQYNICEQMIQIREDHMRFISELARYSNSEVVTGSGRQEAQKTDAEYRKLFDLALQGLQLLSQWSAHVMEVYSWKLVHPTDKYSNKDCPDSAEEYERATRYNYTSEEKFALVEVIAMIKGLQVLMGRMESVFNHAIRHTVYAALQDFSQVTLREPLRQAIKKKKNVIQSVLQAIRKTVCDWETGHEPFNDPALRGEKDPKSGFDIKVPRRAVGPSSTQLYMVRTMLESLIADKSGSKKTLRSSLEGPTILDIEKFHRESFFYTHLINFSETLQQCCDLSQLWFREFFLELTMGRRIQFPIEMSMPWILTDHILETKEASMMEYVLYSLDLYNDSAHYALTRFNKQFLYDEIEAEVNLCFDQFVYKLADQIFAYYKVMAGSLLLDKRLRSECKNQGATIHLPPSNRYETLLKQRHVQLLGRSIDLNRLITQRVSAAMYKSLELAIGRFESEDLTSIVELDGLLEINRMTHKLLSRYLTLDGFDAMFREANHNVSAPYGRITLHVFWELNYDFLPNYCYNGSTNRFVRTVLPFSQEFQRDKQPNAQPQYLHGSKALNLAYSSIYGSYRNFVGPPHFQVICRLLGYQGIAVVMEELLKVVKSLLQGTILQYVKTLMEVMPKICRLPRHEYGSPGILEFFHHQLKDIVEYAELKTVCFQNLREVGNAILFCLLIEQSLSLEEVCDLLHAAPFQNILPRVHVKEGERLDAKMKRLESKYAPLHLVPLIERLGTPQQIAIAREGDLLTKERLCCGLSMFEVILTRIRSFLDDPIWRGPLPSNGVMHVDECVEFHRLWSAMQFVYCIPVGTHEFTVEQCFGDGLHWAGCMIIVLLGQQRRFAVLDFCYHLLKVQKHDGKDEIIKNVPLKKMVERIRKFQILNDEIITILDKYLKSGDGEGTPVEHVRCFQPPIHQSLASS

(19) United States
(12) Patent Application Publication
(10) Pub. No.: US 2024/0139205 A1
(43) Pub. Date: May 2, 2024

(54) MODULATORS OF ARALAR FOR TREATING NEUROLOGICAL DISORDERS

(71) Applicant: Universite de Lausanne, Lausanne (CH)

(72) Inventors: Claudia BAGNI, Blansko (CZ); Tilmann ACHSEL, Paudex (CH); Alexandros KANELLOPOULOS, Montreux (CH)

(21) Appl. No.: 17/905,590

(22) PCT Filed: Mar. 5, 2021
(86) PCT No.: PCT/EP2021/055694

§ 371 Date: Feb. 10, 2023

(30) Foreign Application Priority Data
Mar. 5, 2020 (EP) 20161220.7

(51) Int. Cl. A61K 31/5513; A61K 31/185; A61K 31/4184; A61K 38/13; A61K 45/06; A61P 25/00

(52) U.S. Cl. CPC A61K 31/5513; A61K 31/185; A61K 31/4184; A61K 38/13; A61K 45/06; A61P 25/00

(57) ABSTRACT

The present invention relates to compounds or pharmaceutical compositions for treating neurological disorders. In particular, the invention relates to a modulator of gamma-Aminobutyric acid (GABA) signaling for use in treating a neurological disorder in a subject with Cyfip1 haploinsufficiency. Furthermore, the present invention relates to a modulator of Aralar for use in treating a neurological disorder. The invention further relates to non-medical uses of the compounds or compositions of the invention. Furthermore, the invention relates to a method for identifying a modulator of Aralar and/or a neuroactive drug.

The present invention relates to compounds or pharmaceutical compositions for treating neurological disorders. In particular, the invention relates to a modulator of gamma-Aminobutyric acid (GABA) signaling for use in treating a neurological disorder in a subject with Cyfip1 haploinsufficiency. Furthermore, the present invention relates to a modulator of Aralar for use in treating a neurological disorder. Preferably, the compounds or compositions of the invention are used for treating a neurodevelopmental and/or psychiatric disorder. The invention further relates to the non-medical use of the compounds or compositions of the invention, in particular, for preventing, alleviating, reversing, and/or stopping at least one behavioral deficit. Furthermore, the invention relates to a method for identifying a modulator of Aralar and/or a neuroactive drug, wherein the method comprises (a) contacting Aralar and/or a cell comprising Aralar with a candidate drug; and (b) selecting said candidate drug as Aralar modulator and/or neuroactive drug when the GABA-transporting activity of said Aralar is altered.

Neurodevelopmental disorders are a group of disorders that affect the development of the nervous system, leading to abnormal brain function which may affect emotion, learning ability, self-control, sleep and memory among others. Several neurodevelopmental disorders, such as inter alia autism spectrum disorder (ASD) and schizophrenia (SCZ) can be also classified as psychiatric disorders which are characterized by a behavioral or mental pattern that causes significant distress or impairment of personal functioning.

The development of the nervous system is a complex and tightly regulated dynamic process eventually giving rise to an extremely complex system which includes the brain. Both, genetic and environmental disturbances may lead to pathologic alterations of the neuronal architecture, function or connectivity. A genetic disturbance may be a well-defined genetic variant such as an extended CGG triplet repeat within the Fragile X mental retardation 1 (FMR1) gene found in Fragile X syndrome (FXS), or a rather ill-defined combination of many gene variants whereof each variant is only loosely associated with the disorder, such as in schizophrenia and most forms of autism spectrum disorder. Many neurodevelopmental disorders, e.g. autism spectrum disorder, may be rather considered as multifactorial syndromes which are caused by a large variety of factors and factor combinations. In other words, a certain psychiatric phenotype may have different underlying causes and may be mediated by different mechanisms.

Schizophrenia (SCZ) is thought to have a heritability of about 80%, but candidate gene studies of schizophrenia have generally failed to find consistent associations, and the genetic loci associated with SCZ explain only a small fraction of the variation in the disease (van de Leemput (2016), Adv Genet. 96:99-141; Farrell (2015), Mol Psychiatry 20(5):555-62; Schulz (2016), Schizophrenia and Psychotic Spectrum Disorders, Oxford University Press). Furthermore, also environmental factors such as maternal infection during pregnancy, substance abuse and social factors are thought to play a role. The mechanisms underlying schizophrenia are very complex and thus very poorly understood (van Os (2009), Lancet. 374 (9690): 635-45). A focus has been put on dopamine signaling and glutamate signaling, but GABAergic interneurons are also thought to play a role (Marin (2012), Nature Reviews. Neuroscience. 13 (2): 107-20). Mainly, antipsychotic drugs which modulate the dopaminergic system are so far used for treating schizophrenia. However, the side-effects of those drugs are often severe and include, for example, Parkinson-like symptoms and the development of diabetes.

Autism spectrum disorder (ASD) is thought to have a similarly high heritability as schizophrenia (see e.g. Sandin (2017), JAMA 318(12):1182-1184), and the association of most gene variants with ASD is also weak (Lord (2018), Lancet. 392 (10146): 508-520; Werling (2018), Nature Genetics. 50 (5): 727-736). Only in <30% of cases of ASD a clear genetic cause can be found, for example the above mentioned CGG triplet repeat within the FMR1 gene, which is also linked to ASD and Rett syndrome, Neurofibromatosis, Down Syndrome, Angelman Syndrome among others. Within the ASDEU programme, where 631619 children were tested in 23 regions in 12 European countries from 2015 to 2018, it was found that the average estimated prevalence of autism is 12.2 per 1,000 children aged 7-9 years. However, these rates may be even higher, i.e. about 45 per 1,000 when all forms of autism spectrum disorders such as Fragile X Syndrome, Rett syndrome, Neurofibromatosis, Down Syndrome, Angelman Syndrome among others are included.

Despite the high prevalence, the mechanisms underlying ASD are not completely understood yet, in particular at the molecular level. It is thought that the activities of certain brain regions and their connectivity are altered in individuals with ASD. Furthermore, it has been suggested that ASD could be linked to mitochondrial diseases. There is very little medication available for the treatment of ASD and evidence that such medication is useful is rather weak (Accordino (2016), Expert Opinion on Pharmacotherapy. 17 (7): 937-52).

There have been further attempts to unravel the mechanisms underlying neurodevelopmental or psychiatric disorders such as ASD or SCZ, but a clear understanding is still missing. Converging lines of evidence support a role for mitochondria in brain dysfunctions such as autism spectrum disorder (ASD), schizophrenia (SCZ) and other disabilities featuring synaptic dysfunctions (Castora (2019), Prog Neuropsychopharmacol Biol Psychiatry 92, 83-108; De Rubeis (2014), Nature 515, 209-215; Gandal (2018), Science 359, 693-697; Garcia-Cazorla (2018), J Inherit Metab Dis 41, 909-910; Hollis (2017), Current opinion in neurobiology 45, 178-187. Mitochondrial dysfunction has been observed in ASD with a prevalence of approximately 5-8% (Ghanizadeh (2013), Mitochondrion 13, 515-519; Griffiths (2017), Oxidative medicine and cellular longevity 2017, 4314025; Legido (2013), Semin Pediatr Neurol 20, 163-175; Patowary (2017), Autism Res. 10(8):1338-1343; Rossignol (2012), Molecular psychiatry 17, 290-314; Toker (2015), Neuropsychiatr Dis Treat 11, 2441-2447; Valenti (2014), Neurosci Biobehav Rev 46 Pt 2, 202-217) and in SCZ (Akarsu (2014), J Mol Psychiatry 2, 6.; Taurines (2010), Eur Child Adolesc Psychiatry 19, 441-448). Moreover, mitochondrial genes have been reported to be differentially expressed in the cerebral cortex of ASD patients and strongly correlated with genes associated with synaptic transmission (Schwede (2018), J Neurodev Disord 10, 18). In agreement, a GWAS study showed a significant enrichment for ASD and SCZ-associated common variants in genes that regulate synapse & mitochondrial homeostasis (Gandal (2018), Science 359, 693-697). Whereas these studies suggest a potential association between energetic balance, synaptic transmission, and psychiatric disease, the mechanistic links between these processes remain entirely unclear. The absence of a mechanistic understanding precludes the identification of promising drug targets and the development of effective and safe therapies.

In a further line of evidence, Cyfip1 mutations, i.e. copy number variations (CNVs) and single nucleotide variants (SNVs) in the Cyfip1 gene have been associated with schizophrenia and ASD (Leblond (2012), PLoS Genet 8, e1002521; Stefansson (2014), Nature 505, 361-366; Tam (2010), Biochem Soc Trans 38, 445-451; Vanlerberghe (2015), Eur J Med Genet 58, 140-147; Wang (2015), Ann Hum Genet. 79(5):329-340; Zhao (2013), Schizophr Bull 39, 712-719; Williams (2019), bioRxiv, 722504; Angulo (2015), Journal of Endocrinological Investigation 38(12):1249-1263; van der Zwaag B (2010), Am J Med Genet B Neuropsychiatr Genet. 153B(4):960-6; Purcell (2014), Nature 506(7487):185-90; Butler (2017), J Intellect Disabil Res. 61(6):568-579; Peng (2018), CNS Neurosci Ther. 24(12):1196-1206; Toma (2014), Mol. Psychiatry, 19:784-790). Furthermore, genomic instability at the 151.2 BP1-BP2 locus, which encompasses four genes including the Cytoplasmic FMR1 interacting protein 1 (Cyfip1), has also recently emerged as a recognized syndrome (Cafferkey (2014), Am J Med Genet A 164A, 1916-1922; Cox (2015), International journal of molecular sciences 16, 4068-4082; De Wolf (2013), Am J Med Genet A 161A, 2846-2854; Nevado (2014), Genet Mol Biol 37, 210-219; Urraca (2013), Autism Res. 6, 268-279. Among the four implicated genes, converging evidence suggest that Cyfip1 is a key factor mediating risk for the BP1-2-deletion disorders (Das (2015), Molecular neuropsychiatry 1, 116-123; Nebel (2016), PloS one 11, e0148039; Vanlerberghe (2015), Eur J Med Genet 58, 140-147; Wang (2015), Ann Hum Genet. 79(5):329-340; Woo (2016), PloS one 11, e0158036; Yoon (2014), Cell stem cell 15, 79-91). Moreover, recent studies demonstrated that Cyfip1 haploinsufficiency in humans, mice and flies confers domain-specific cognitive impairments and behavioral deficits, i.e. ASD- and/or SCZ-like behaviors (Bachmann (2019), Translational psychiatry 9, 29; Bozdagi (2012), PloS one 7, e42422; Dominguez-Iturza (2019), Nature Communications 10, 3454; Silva (2019), Nature Communications 10, 3455; Woo (2019), Biol Psychiatry. 2019 Aug. 15; 86(4):306-314). A further recent study has shown that copy-number variations at the 15q11.2 BP1-BP2 locus are present in 0.5 to 1.0% of the human population, and the respective deletion is associated with a range of neurodevelopmental disorders (Silva (2020), bioRxiv preprint doi: https://doi.org/10.1101/2020.09.03.280859; version posted Sep. 3, 2020). However, the molecular mechanisms linking Cyfip1 deficiency with alterations in behavior and cognition are unknown. Although a relatively clear association of one gene with a complex disease such as ASD or SCZ is an important finding by itself, the absence of a mechanistic understanding precludes the development of targeted therapies for specific patient groups. Due to the complexity and various sub-forms of neurodevelopmental and/or psychiatric disorders such as ASD or SCZ, it is desirable to tailor the treatment regime to specific patient groups.

In summary, certain gene variants, brain regions, neuronal signaling and mitochondria have been implicated in the development and manifestation of neurological diseases, i.e. ASD and schizophrenia, but an understanding of the mechanistic links between those features is missing. This is mirrored in the very limited availability of effective and safe medication for the treatment of highly complex neurological disorders, i.e. for ASD and schizophrenia.

Thus, there is still a need for molecular targets associated with certain neurological disorders, means for treating neurological disorders and/or alleviating behavioral deficits, in particular in specific subject groups, and methods for identifying such means.

The technical problem is solved by the embodiments provided herein and as characterized in the claims.

### DETAILED DESCRIPTION

Accordingly, the invention relates to a modulator of gamma-Aminobutyric acid (GABA) signaling for use in treating a neurological disorder in a subject with Cyfip1 haploinsufficiency, preferably wherein said modulator enhances GABA signaling. Preferably, said modulator of GABA signaling is a modulator of Aralar, as described herein.

Thus, the present invention also relates to a modulator of Aralar for use in treating a neurological disorder.

The invention is, at least partly, based on the surprising discovery, as illustrated in the appended Examples, that sequestration of gamma-Aminobutyric acid (GABA) in the mitochondria of the brain regulates behavior. In particular, the inventors established a direct link how mitochondrial membrane polarization altered GABA signaling through Aralar, leading to deficits in behavior, in particular in subjects with Cyfip1 haploinsufficiency. So far, no mitochondrial GABA carrier has been identified in animals or humans. The inventors surprisingly found that Aralar is the major mitochondrial transporter which regulates the sequestration of GABA into mitochondria. Thus, the inventors further found a novel mechanism through which the GABA levels are altered, for example, in subjects with Cyfip1 haploinsufficiency. Aralar has been known as an aspartate/glutamate carrier, but its GABA-transporting activity has never been reported (Amoedo (2016), Biochim Biophys Acta 1863, 2394-2412; Palmieri (2013), Mol Aspects Med 34, 465-484). In fact, many mechanistic and behavioral experiments, as illustrated in the appended Examples, were required to detect this surprising novel function of Aralar. Further surprisingly, it was found that modulation, i.e. inhibition, of the Aralar activity by applying an exemplary small molecule (Pyridoxal 5′-phosphate hydrate), by decreasing the mitochondrial membrane potential through IDH inhibition (i.e. by ML309), or by genetically reducing Aralar levels normalized the behavior in an accepted animal model for autism spectrum disorder (ASD) and schizophrenia. The inventors thus found that Aralar is a molecular target for the treatment of ASD and/or SCZ and/or neurological disorders that are associated with altered/defective GABA homeostasis. The surprising identification of Aralar as mitochondrial GABA-transporter in the brain also enables drug screening/the screening of molecules, i.e. candidates for neuroactive drugs, which are tested for their ability to modulate the GABA-transporting activity of Aralar and/or a phenotype which is associated with the GABA-transporting activity of Aralar (i.e. GABA levels/localization).

Thus, the invention further relates a method for identifying a modulator of Aralar and/or a neuroactive drug, wherein the method comprises
(a) contacting Aralar and/or a cell comprising Aralar with a candidate drug; and
(b) selecting said candidate drug as Aralar modulator and/or neuroactive drug when the GABA-transporting activity of said Aralar is altered.

The inventive screening methods provided herein are useful for identifying novel modulators of Aralar and to test molecules for their potential use in the treatment of a neurological disorder (neuroactive drugs) as described herein, in particular a neurodevelopmental and/or psychiatric disorder such as inter alia ASD and/or SCZ. Said screening methods are not particularly limited to certain molecules as drug candidates. Drug candidates may have been, for example, never been used in therapy or have been proven useful in the treatment of a certain disease. Thus, the screening methods according to the invention allow identifying molecules as Aralar inhibitors/neuroactive drugs which can then be further tested for their use in the treatment of a neurological disorder according to the invention.

In the context of the invention, and as used herein, the term behavior (or equivalents thereof), refers, in particular, to social behavior and the level of repetitive behavior as described herein. A normal behavior thus refers to a normal social behavior/interaction and a low level of repetitive behavior (herein considered as absence of repetitive behavior). Furthermore, a normal behavior may further refer to a normal cognitive function/ability. As detailed out further below, behavioral deficits (deficits in behavior) comprise repetitive behavior, deficits in social behavior and/or cognitive deficits. Thus, abnormal/aberrant social behavior is also described herein by the term “deficit(s) in social behavior”. Furthermore, a high level of repetitive behavior is considered as a behavioral deficit and is described herein simply by the term “repetitive behavior”. Abnormal, i.e. reduced, cognitive function/ability is described herein by the term “cognitive deficit(s)”.

GABA, also termed gamma-aminobutyric acid or γ-aminobutyric acid, is the main inhibitory neurotransmitter in the developmentally mature mammalian central nervous system. Its principal role is reducing neuronal excitability throughout the nervous system. Neurons that produce GABA as their output are called GABAergic neurons. In vertebrates, GABA acts at inhibitory synapses in the brain by binding to specific transmembrane receptors in the plasma membrane of both pre- and postsynaptic neuronal processes. This binding causes the opening of ion channels to allow the flow of either negatively charged chloride ions into the cell or positively charged potassium ions out of the cell. This action results in a negative change in the transmembrane potential, usually causing hyperpolarization. Two general classes of GABA receptor are known: GABAA in which the receptor is part of a ligand-gated ion channel complex, and GABAB metabotropic receptors, which are G protein-coupled receptors that open or close ion channels via intermediaries (G proteins).

A term denoting a certain gene, mRNA, or protein may be used herein for denoting the gene, mRNA and/or protein of said gene/mRNA/protein. It has no particular meaning herein, if such a term is written in minor or capital letter or a mix of both, i.e. it is not restricted to a certain species by the format. If said term is written in italics, it refers in particular to the gene or the mRNA, but this is not a requirement for denoting a gene or mRNA. As an example, to illustrate this general principle, Aralar and ARALAR are used interchangeably herein and may refer to the Aralar gene, mRNA or protein of inter alia flies, mice and humans, whereas Aralar refers in particular to the Aralar gene or mRNA, also regardless of the species.

Aralar, as used herein, refers in vertebrates, i.e. mouse and human, to Aralar1 (also known as Slc25a12 or Agc1) and Aralar2 (also known as Slc25a13, Agc2 or Citrin), and in flies, i.e. Drosophila, it refers to Aralar1 (Fly gene ID 43616).

Human Aralar1 (Slc25a12; solute carrier family 25 member 12) has the gene ID 8604. In particular said human Aralar1 has the nucleotide sequence SEQ ID NO:1 and/or the protein sequence SEQ ID NO:2.

Human Aralar2 (Slc25a13; solute carrier family 25 member 13) has the gene ID 10165. In particular, said human Aralar2 has the nucleotide sequence SEQ ID NO:3 and/or the protein sequence SEQ ID NO:4.

Aralar is an integral membrane protein located in the inner mitochondrial membrane known as a mitochondrial aspartate-glutamate carrier (Age) which exports an aspartate from the mitochondria in exchange for a glutamate and a proton in the cytosol. Aralar further participates in the cellular redox homeostasis and calcium-mediated control of mitochondrial respiration via the malate-aspartate shuttle. The kinetic properties of Aralar have been extensively studied in proteoliposomes from the liver. In the context of the present invention, it has been surprisingly found that Aralar further regulates the transport of GABA from the cytosol into the mitochondria, in particular by making use of a proton gradient across the mitochondrial membrane. Thus, in the context of the present invention, Aralar is a GABA-transporter and/or has a GABA-transporting activity. Moreover, in the context of the present invention, the activity of Aralar refers, in particular, to the transport of GABA from the cytosol into mitochondria. The function as GABA-transporter and/or the GABA-transporting activity, i.e. the regulation of mitochondrial versus cytosolic/synaptic GABA levels by Aralar, may be direct or indirect. However, since Aralar is a mitochondrial transport protein (carrier), the GABA-transporting activity of Aralar is, in particular, direct, which means that Aralar transports GABA directly from the cytosol into the mitochondria.

The mitochondrion (plural mitochondria) is a double-membrane-bound organelle found in most eukaryotic organisms. Mitochondria generate most of the cell's supply of adenosine triphosphate (ATP), used as a source of chemical energy. In addition to supplying cellular energy, mitochondria are involved in other tasks, such as signaling and cellular differentiation. The mitochondrial compartments include the outer membrane, the intermembrane space, the inner membrane, and the cristae and matrix. The outer and inner membranes of mitochondria are composed of phospholipid bilayers and proteins. The two membranes have different properties. The outer membrane is freely permeable to small molecules, thus the concentrations of small molecules in the intermembrane space is the same as in the cytosol but the protein composition of this space is different from the protein composition of the cytosol. The inner mitochondrial membrane contains proteins with five types of functions, inter alia those that perform the redox reactions of oxidative phosphorylation, the ATP synthase and specific transport proteins that regulate metabolite passage into and out of the mitochondrial matrix. A dominant role for the mitochondria is the production of ATP. This involves the oxidization of major products of glucose: pyruvate, and NADH, which are produced in the cytosol. This type of cellular respiration known as aerobic respiration. Pyruvate molecules produced by glycolysis are actively transported across the inner mitochondrial membrane, and into the matrix where they can be oxidized and combined with coenzyme A to form C02, acetyl-CoA, and NADH. Acetyl-CoA is the only fuel to enter the TCA cycle (tricarboxylic acid cycle), also known as citric acid cycle or the Krebs cycle. The TCA cycle is a series of chemical reactions used by all aerobic organisms to release stored energy through the oxidation of acetyl-CoA into ATP and carbon dioxide. In the TCA cycle, all the intermediates (e.g. citrate, iso-citrate, alpha-ketoglutarate, succinate, fumarate, malate and oxaloacetate) are regenerated during each turn of the cycle. Adding more of any of these intermediates to the mitochondrion therefore means that the additional amount is retained within the cycle, increasing all the other intermediates. Hence, the addition of any one of them to the cycle has an anaplerotic effect, and its removal has a cataplerotic effect. This in turn increases or decreases the rate of ATP production by the mitochondrion. Specifically, the TCA cycle oxidizes the acetyl-CoA to carbon dioxide, and, in the process, produces reduced cofactors (three molecules of NADH and one molecule of FADH2) that are a source of electrons for the electron transport chain, and a molecule of GTP (that is readily converted to an ATP). Furthermore, reducing equivalents from the cytoplasm can be imported via the malate-aspartate shuttle system of antiporter proteins. The redox energy from NADH and FADH2 is transferred to oxygen in several steps via the electron transport chain. This energy is used to pump protons (H+) into the intermembrane space. As the proton concentration increases in the intermembrane space, a strong electrochemical gradient is established across the inner membrane. The protons can return to the matrix through the ATP synthase complex, and their potential energy is used predominantly to synthesize ATP from ADP and inorganic phosphate. Damage and subsequent dysfunction in mitochondria is an important factor in a range of human diseases due to their influence in cell metabolism. Mitochondrial disorders often present themselves as neurological disorders, including inter alia autism, schizophrenia, bipolar disorder, dementia, Alzheimer's disease, Parkinson's disease, epilepsy, stroke and chronic fatigue syndrome.

The terms “cytosol” and “cytoplasm” are used interchangeably herein and refer to the content of a cell inside the plasma membrane except the nucleus/nuclei and the mitochondria. Thus, the cytosol or cytoplasm, as used herein, comprises inter alia the aqueous phase of a cell including the dissolved molecules, the cell skeleton, ribosomes, all organelles except mitochondria, vesicles and phase-separated condensates. A vesicle is a structure within or outside a cell, consisting of liquid or cytoplasm enclosed by a lipid bilayer. Vesicles form naturally during the processes of secretion (exocytosis), uptake (endocytosis) and transport of materials within the plasma membrane. Synaptic vesicles occur in neurons and store various neurotransmitters, such as inter alia GABA, that are released at the synapse. Synaptic vesicles are essential for propagating nerve impulses between neurons and are constantly recreated by the cell. Synaptic vesicles typically have the shape of a sphere of about 40 nm in diameter and consist largely of phospholipids and proteins, i.e. transport proteins involved in neurotransmitter uptake, and trafficking proteins that participate in synaptic vesicle exocytosis, endocytosis, and recycling.

It is evident that the cytosol or mitochondria of the brain, as used herein, refers to the cytosol or mitochondria, respectively, of brain cells, i.e. neurons.

In humans, Slc25a12 (Aralar1) and Slc25a13 (Aralar2) are both expressed in the brain, but Slc25a12 shows higher expression than Slc25a13. In mouse, Slc25a12 is strongly expressed in the brain, whereas Slc25a13 is very poorly expressed in the brain. Slc25a12 is central to neuronal physiology and also plays a role in glutamate-induced excitotoxicity. Mutations in the Slc25a12 gene cause early infantile epileptic encephalopathy 39 (EIEE39), whereas Slc25a13 is linked to neonatal intrahepatic cholestasis caused by citrin deficiency (NICCD). Aralar is overexpressed in several types of human tumors.

In the context of treating a neurological disorder according to the invention, the Aralar is preferably human Aralar because preferably a human subject is treated. Preferably, said human Aralar is Slc25a12, at least because Slc25a12 is highly expressed in the human brain and associated with neuronal physiology and a rare neurological disease. The same applies to the inventive non-medical uses of the modulator of Aralar as provided herein.

In the context of the inventive screening methods provided herein, Aralar may be human Aralar or Aralar of a non-human animal such as inter alia a mouse or a fly.

As used herein, Aralar may also refer to a functional fragment thereof, such as inter alia a fragment according to the sequence SEQ ID NO:10 or SEQ ID NO:11, wherein the functional fragment functions as GABA-transporter.

The skilled person has no difficulties in identifying an orthologue of a gene/protein in different species, for example, by interrogating well known databases such as inter alia Ensembl, Pubmed and/or Genome Browser. In particular, using those databases it is possible to identify an Aralar sequence of another species, such as inter alia, a mouse, a rat, a chimpanzee, a monkey, a fish, a frog, or a fly, which is orthologous to the human Aralar protein sequence. Furthermore, an Aralar protein sequence that is orthologous to the human Aralar protein sequence may be identified/validated by determining the sequence identity to the human Aralar protein sequence as described herein.

In particular, as used herein in the context of the inventive screening methods provided herein, Aralar refers to
(i) a human Aralar protein sequence according to SEQ ID NO:2 or SEQ ID NO:4, preferably SEQ ID NO:2;
(ii) a nucleotide sequence encoding human Aralar according to SEQ ID NO:1 or SEQ ID NO:3, preferably SEQ ID NO:1;
(iii) an Aralar protein sequence which is orthologous to said human Aralar protein sequence in (i);
(iv) a nucleotide sequence encoding Aralar which is orthologous to said nucleotide sequence encoding human Aralar in (ii);
(v) a protein sequence encoding a functional fragment of human Aralar with the sequence according to SEQ ID NO:10 or SEQ ID NO:11, preferably SEQ ID NO:10;
(vi) a protein sequence encoding a functional fragment of Aralar which is homologous/orthologous to said protein sequence encoding a functional fragment of human Aralar;
(vii) a protein sequence with at least 50%, 60%, 70%, 80% or 90%, preferably at least 90%, 95%, 98% or 99%, preferably 95%, 96%, 97%, 98%, or 99% sequence identity to the protein sequence of (i), (iii), (v) or (vi) or
(viii) a nucleotide sequence encoding a protein with the sequence according to (vii).

In certain embodiments, in particular in the context of the cell-based and/or in vitro assay-based screening methods provided herein, Aralar refers to the human Aralar protein sequence according to SEQ ID NO:2 or SEQ ID NO:4, preferably SEQ ID NO:2, and/or a protein sequence with at least 50%, 60%, 70%, 80% or 90%, preferably at least 90%, 95%, 98% or 99%, preferably 95%, 96%, 97%, 98%, or 99% sequence identity to said human Aralar protein sequence.

With regard to the functional fragments of Aralar, further reference is made to Palmieri (2001), EMBO J. 20(18):5060-9.

In any case, it may be validated that the Aralar protein used in the inventive screening methods provided herein has a GABA-transporting activity, for example, by employing a known (direct or indirect) modulator of GABA (such as inter alia Pyridoxal 5′-phosphate, ML309 or oligomycin) and a corresponding assay as described herein, for example, an assay employed in a screening method of the invention or an assay described herein for evaluating whether a compound/molecule is an inhibitor or activator of Aralar.

As used herein, the term “sequence identity” is used to describe the sequence relationships between two or more amino acid sequences, proteins (or fragments thereof), or polypeptides (or fragments thereof). The term can be understood in the context of and in conjunction with the terms including: (a) reference sequence, (b) comparison window, (c) sequence identity, (d) percentage of sequence identity, and (e) substantial identity or “homologous”.

A “reference sequence” is a defined sequence used as a basis for sequence comparison. A reference sequence may be a subset of or the entirety of a specified sequence.

A “comparison window” includes reference to a contiguous and specified segment of a amino acid sequence/polypeptide sequence/protein sequence, wherein the amino acid sequence/polypeptide sequence/protein sequence may be compared to a reference sequence. The portion of the amino acid sequence/polypeptide sequence/protein sequence in the comparison window may comprise additions, substitutions, or deletions (i.e., gaps) compared to the reference sequence (which does not comprise additions, substitutions, or deletions) for optimal alignment of the two sequences. Generally, the comparison window may be at least about 3 amino acid residues in length, and optionally can be about 3, 4, 5, 6, 7, 8, 9, 11, 13, 16, or 33 amino acid residues in length or longer. Those of skill in the art understand that to avoid a misleadingly high similarity to a reference sequence due to inclusion of gaps in the polynucleotide or polypeptide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wisc., USA; the CLUSTAL program is well described by Higgins and Sharp (1988) Gene 73: 237-244; Corpet et al. (1988) Nucleic Acids Research 16:881-90; Huang, et al. (1992) Computer Applications in the Biosciences, 8:1-6; and Pearson, et al. (1994) Methods in Molecular Biology, 24:7-331. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs, or their successors, using default parameters. Altschul et al. (1997) Nucleic Acids Res, 2:3389-3402. It is to be understood that default settings of these parameters can be readily changed as needed in the future. Evidently, for comparison of amino acid sequences/protein sequences/polypeptide sequences, an algorithm/program directed to the alignment of amino acid sequences/protein sequences/polypeptide sequences should be used, i.e. BLASTP. As those ordinary skilled in the art will understand, BLAST searches assume that proteins or nucleic acids can be modeled as random sequences. However, many real proteins and nucleic acids comprise regions of nonrandom sequences which may be homopolymeric tracts, short-period repeats, or regions enriched in one or more amino acids or nucleic acids. Such low-complexity regions may be aligned between unrelated proteins even though other regions of the protein or nucleic acid are entirely dissimilar. A number of low-complexity filter programs can be employed to reduce such low-complexity alignments. For example, the SEG (Wooten et al. (1993) Comput. Chem. 17:149-163) and XNU (Claverie et al. (1993) Comput. Chem. 17:191-1) low-complexity filters can be employed alone or in combination.

“Sequence identity” in the context of two polypeptide/protein sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

“Percentage of sequence identity” means the value determined by comparing two optimally aligned sequences over a comparison window, wherein the portion of the amino acid/peptide/protein sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

As detailed out in the appended Examples, the inventors provide for the first time a mechanistic link between Aralar and behavior associated with autism spectrum disorder. It has been notably found in the context of the present invention that in an accepted fly model for ASD and SCZ (Drosophila Cyfip^{85.1}/+ showing behavioral deficits; Example 3), GABA levels are sequestered in the mitochondria of the brain (Example 7) due to the action of Aralar (Example 9), and that direct and indirect inhibition of Aralar normalized the behavior of those flies (Examples 5 and 10). The establishment of a causative role of GABA sequestration by Aralar leading to behavioral deficits in an animal having a mutation in the Cyfip1 gene which is associated with autism spectrum disorder in humans, and said animal showing behavioral deficits which are reminiscent of ASD, is a surprising finding and a highly useful contribution to the art, especially for the development of therapies for the treatment of neurological disorders, in particular ASD or SCZ. The person skilled in the art understands that a purely correlative, suggestive and entirely uncertain link between Aralar and autism as has been described in the prior art is, especially in the absence of any mechanistic understanding, not very useful for developing such therapies.

The terms “disorder” and “disease” are used herein interchangeably and refer to a pathophysiological response to external or internal factors, a disruption of normal or regular functions in the body or a part of the body and/or an abnormal state of health that interferes with the usual activities or feeling of wellbeing. As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

A neurological disorder, as used herein, refers to a disorder of the nervous system, preferably a disorder of the central nervous system, in particular the brain. Preferably, herein and in the context of the invention, the neurological disorder is a neurodevelopmental disorder, a psychiatric disorder, a disorder associated with synaptic dysfunction and/or a disorder associated with behavioral deficits.

A neurodevelopmental disorder is a disorder which affects the development of the nervous system, leading to abnormal brain function which may affect emotion, learning ability, self-control, and memory. A neurodevelopmental disorder is, in particular, an intellectual disability/mental retardation/disability; a learning disorder such as inter alia Dyslexia or Dyscalculia; an autism spectrum disorder, such as inter alia Asperger's syndrome or Autistic Disorder; a motor disorder such as inter alia developmental coordination disorder and stereotypic movement disorder; a tic disorder such as inter alia Tourette's syndrome; a traumatic brain injury such as inter alia cerebral palsy; a communication, speech and language disorder; a predominantly genetic disorders, such as inter alia fragile-X syndrome, Down syndrome, attention deficit hyperactivity disorder, schizophrenia, schizotypal disorder and hypogonadotropic hypogonadal syndromes; and/or a disorder due to a neurotoxicant such as inter alia fetal alcohol spectrum disorder, Minamata disease and a behavioral disorder caused by heavy metals, dioxin, PBDEs and PCBs, medications and/or illegal drugs.

A psychiatric disorder, also called mental disorder or mental illness is a behavioral or mental pattern that causes significant distress or impairment of personal functioning. Such features may be persistent, relapsing and remitting, or occur as a single episode. A psychiatric disorder is, in particular, an anxiety disorder such as inter alia a specific phobia, generalized anxiety disorder, social anxiety disorder, panic disorder, obsessive-compulsive disorder and post-traumatic stress disorder, a mood disorder such as inter alia unipolar depression, or bipolar disorder, a psychotic disorder such as inter alia schizophrenia and delusional disorder, a personality disorder, an eating disorder such as inter alia anorexia nervosa or bulimia nervosa, a sleep disorder, an impulse control disorder such as inter alia kleptomania or pyromania, a behavioral addiction such as inter alia gambling addiction, a substance use disorder, a dissociative disorder such as inter alia depersonalization disorder or dissociative identity disorder, a cognitive disorder such as inter alia delirium and mild and major neurocognitive disorder/dementia, a developmental disorders such as inter alia autism spectrum disorder, oppositional defiant disorder and conduct disorder, antisocial personality disorder and attention deficit hyperactivity disorder, and/or a factitious disorder such as inter alia Munchausen syndrome. Preferably, in the context of the invention a psychiatric disorder is schizophrenia, autism-spectrum disorder and/or obsessive-compulsive disorder.

A disorder associated with synaptic dysfunction, as used herein, comprises disabilities featuring synaptic dysfunctions and synaptopathies. The term “disabilities featuring synaptic dysfunctions” includes diseases/disorders that are associated and/or caused by a mutation of gene encoding a synaptic protein, such as inter alia Episodic and Paroxysmal syndromes, Systemic atrophies primarily affecting the central nervous system, Cerebral palsy and other paralytic syndromes, Extrapyramidal and movement disorders, and/or degenerative diseases of the nervous system, in particular inter alia autism, epilepsy, Parkinson's disease, Alzheimer's disease, leigh syndrome, Huntington's disease, ataxia, amyotrophic lateral sclerosis (Grant (2012), Curr Opin Neurobiol. 22(3):522-9; Bayes (2011), Nat Neurosci. 14(1):19-21), diseases/disorders that are associated and/or caused by altered synaptic structure and/or function (synaptopathies) such as inter alia fragile X syndrome (FXS), autism spectrum disorders (ASDs), Schizophrenia (SCZ) and other intellectual disabilities (IDs) (Bagni (2019), Neuron 101(6):1070-1088; Wang (2018), Genome Med. 10(1):9), and diseases/disorders that are associated and/or caused by an aberrant synapse physiology such as inter alia autism, Down syndrome, startle disease, and epilepsy, and neurodegenerative disorders (i.e. Alzheimer and Parkinson disease) (Lepeta (2016), J Neurochem. 138(6):785-805).

Furthermore, a synaptopathy is a disease of the brain, spinal cord or peripheral nervous system relating to the dysfunction of synapses. In particular, a synaptopathy is a channelopathy such as inter alia episodic ataxia; an autoimmune synaptopathy such as inter alia Myasthenia gravis; autism spectrum disorder; schizophrenia; and/or a neurodegenerative disorder such as inter alia Alzheimer's disease. In the context of the invention, a disorder associated with synaptic dysfunction preferably comprises the dysfunction of a GABAergic synapse, in particular in the central nervous system, preferably the brain.

As used herein in the context of the invention, behavioral deficits (deficits in behavior) refer to (i) repetitive behavior, (ii) deficits in social behavior, and/or (iii) cognitive deficits as described in the following. The terms “behavioral deficits” and “deficits in behavior” are used interchangeably herein. In certain embodiments, behavioral deficits refer to (i) repetitive behavior and/or (ii) deficits in social behavior.

Repetitive behavior, as used herein, refers, in particular, to restricted, repetitive patterns of behavior, interests, or activities, for example inter alia the following: stereotyped or repetitive motor movements, use of objects, or speech (e.g., simple motor stereotypes, lining up toys or flipping objects, echolalia, idiosyncratic phrases); insistence on sameness, inflexible adherence to routines, or ritualized patterns of verbal or nonverbal behavior (e.g., extreme distress at small changes, difficulties with transitions, rigid thinking patterns, greeting rituals, need to take same route or eat same food every day; restricted, fixated interests that are abnormal in intensity or focus (e.g., strong attachment to or preoccupation with unusual objects, excessively circumscribed or perseverative interests); and/or hyper- or hyporeactivity to sensory input or unusual interest in sensory aspects of the environment (e.g., apparent indifference to pain/temperature, adverse response to specific sounds or textures, excessive smelling or touching of objects, visual fascination with lights or movement).

Deficits in social behavior, as used herein, are, in particular, difficulties creating connections with other people, social isolation, abnormal responses to sensations, reduced social competence, lack of desire to form relationships, problems with friendships or romantic relationships, atypical patterns of behavior and communication, poor nonverbal communication skills such as lack of eye contact and meaningful gestures and facial expressions, repetitive or rigid language, lack of social awareness, reduced social engagement, speaking in lesson-like monologues, a decreased ability to understand reality, false beliefs, unclear or confused thinking, hearing voices that do not exist, reduced emotional expression, lack of motivation, little emotion inability to experience pleasure, deficits in social-emotional reciprocity (ranging, for example, from abnormal social approach and failure of normal back-and-forth conversation; to reduced sharing of interests, emotions, or affect; to failure to initiate or respond to social interactions), deficits in nonverbal communicative behaviors used for social interaction (ranging, for example, from poorly integrated verbal and nonverbal communication; to abnormalities in eye contact and body language or deficits in understanding and use of gestures; to a total lack of facial expressions and nonverbal communication), and/or deficits in developing, maintaining, and understanding relationships (for example, difficulties adjusting behavior to suit various social contexts).

Cognitive deficits, as used herein, are, in particular, impaired theory of mind, impaired joint attention, strange speech, poverty of speech, impaired memory (i.e. working memory, long-term memory, episodic memory and/or verbal declarative memory, impaired semantic processing, reduced attention, impaired learning. Theory of mind is the ability to attribute mental states, e.g. beliefs, intents, desires, emotions, knowledge to oneself, and to others, and to understand that others have beliefs, desires, intentions, and perspectives that are different from one's own. Theory of mind is crucial for everyday human social interactions and is used when analyzing, judging, and inferring others' behaviors. Joint attention is the shared focus of two individuals on an object. It is achieved when one individual alerts another to an object by means of eye-gazing, pointing or other verbal or non-verbal indications.

Preferably, herein and in the context of the invention, i.e. in the context of the inventive medical uses of a modulator of GABA signaling such as an Aralar modulator (i.e. of an Aralar inhibitor) as provided herein, the neurological disorder is a neurodevelopmental disorder, a psychiatric disorder and/or a disorder that is associated with synaptic dysfunction, wherein said neurological disorder is accompanied by behavioral deficits. Preferably, said behavioral deficits comprise at least one, preferably at least 2, preferably at least 4, preferably at least 8 of the group consisting of: repetitive behavior; reduced social competence; atypical patterns of behavior and communication; poor nonverbal communication skills such as lack of eye contact and meaningful gestures and facial expressions; repetitive or rigid language and/or strange or poor speech; a decreased ability to understand reality; reduced emotional expression; lack of motivation; impaired theory of mind; impaired memory; and/or impaired learning. More preferably, said behavioral deficits comprise (i) reduced social competence and/or (ii) repetitive behavior.

Preferably, herein and in the context of the invention, i.e. in the context of the inventive medical uses of a modulator of GABA signaling such as an Aralar modulator (i.e. of an Aralar inhibitor) as provided herein, the neurological disorder is a neurodevelopmental disorder and/or a psychiatric disorder that is associated with a synaptic dysfunction. Preferably, said synaptic dysfunction involves a GABAergic synapse, preferably in the brain. Even more preferably, the neurological disorder according to the invention is said neurodevelopmental disorder and/or psychiatric disorder which is accompanied by said behavioral deficits and associated with said synaptic dysfunction. In particular, said neurodevelopmental disorder and/or psychiatric disorder which is accompanied by said behavioral deficits and associated with said synaptic dysfunction, is ASD, SCZ, FXS, obsessive-compulsive disorder, and/or a disability featuring synaptic dysfunctions, preferably wherein the disability featuring synaptic dysfunctions is Schizophrenia, Epilepsy, Down Syndrome, Angelman syndrome, Alzheimer's disease and/or Parkinson's disease.

Most preferably, the neurological disorder according to the invention, i.e. in the context of the inventive medical uses of a modulator of GABA signaling such as an Aralar modulator (i.e. of an Aralar inhibitor) as provided herein, is autism spectrum disorder, schizophrenia and/or obsessive-compulsive disorder, in particular autism spectrum disorder. Preferably, said autism spectrum disorder is fragile X syndrome or is associated with fragile X syndrome. As illustrated in the appended Example 12, decreasing Aralar levels or the activity of Aralar ameliorates or normalizes the deficits in social behavior of a fragile X syndrome fly model.

Autism spectrum disorder (ASD), also known as autism spectrum, is a range of mental disorders of the neurodevelopmental type. It includes autism and Asperger syndrome. Individuals on the spectrum often experience difficulties with social communication and interaction and restricted, repetitive patterns of behavior, interests, or activities. According to a redefinition by the DSM-5, autism spectrum disorders encompass the previous diagnoses of autism, Asperger syndrome, pervasive developmental disorder not otherwise specified (PDD-NOS), and childhood disintegrative disorder. Typical symptoms of ASD comprise problems with communication and/or social interaction, restricted interests and repetitive behavior.

Fragile X syndrome (FXS) is a genetic disorder which is often accompanied by a mild to moderate intellectual disability. About a third of those affected have features of autism such as problems with social interactions and delayed speech. FXS is typically caused by an expansion of the CGG triplet repeat (>200 repeats) within the Fragile X mental retardation 1 (FMR1) gene on the X chromosome. The mutation results in the absence or mutated fragile X mental retardation protein (FMRP), which is required for normal brain development and proper neuronal connections. Fragile X syndrome is the most frequent monogenetic form of autism. Even if FXS and autism are not co-diagnosed, subjects with FXS have a high incidence of ASD (Bagni (2019) Neuron, 101(6):1070-1088).

Schizophrenia is a mental illness characterized by abnormal behavior, strange speech, and a decreased ability to understand reality. Other symptoms may include false beliefs, unclear or confused thinking, hearing voices that do not exist, reduced social engagement and emotional expression, and lack of motivation. Schizophrenia if often accompanied by social withdrawal, sloppy dress and poor hygiene, and/or loss of motivation and judgment. Also, symptoms of paranoia may occur, as well as difficulties in working and long-term memory. Furthermore, deficits in cognitive abilities are a core feature of schizophrenia.

As illustrated in the appended Examples, the GABA signaling is altered in Cyfip/Cyfip1 haploinsufficient (Cyfip^{85.1}/+) flies that are used as a model for ASD and/or SCZ, and also in Cyfip1 haploinsufficient (Cyfip1^{+/-}) mice. In particular, the inventors surprisingly found that the deficits in behavior of Cyfip^{85.1}/+ flies are mediated by GABAergic neurons (Example 6), and that the augmentation of GABA levels/signaling, i.e. in brain neurotransmission, surprisingly normalized said deficits in behavior (Example 8). In addition, it was surprisingly found that augmentation of GABA signaling by Diazepam, a GABA-A positive modulator, normalized deficits in social behavior also in Cyfip1^{+/-} mice. Thus, in the context of the present invention, a direct link between altered GABA signaling and deficits in behavior, in particular in subjects with Cyfip1 haploinsufficiency has been established. Moreover, the deficits in behavior observed in flies and mice relate to deficits in behavior observed in patients with ASD and schizophrenia.

The behavioral assays with flies and mice described in the appended Examples are suitable for screening, testing and/or validating the efficacy of a candidate drug/molecule for use as a neuroactive drug (modulator of Aralar), i.e. for treating a neurological disorder, such as inter alia ASD, SCZ or Obsessive-Compulsive Disorder (OCD), as provided herein. In particular, as illustrated in Examples 2 and 3 (and Example 14), the deficits in behavior of the Cyfip^{85.1}/+ ASD/SCZ fly model are remarkably similar to the symptoms, i.e. the deficits in behavior, of human patients suffering from neurodevelopmental and/or psychiatric disorders such as ASD, SCZ or OCD. Patients suffering from a neurological disorder as described herein, i.e. ASD, SCZ or OCD-patients, i.e. ASD-patients, may experience difficulties with social communication, may have reduced social competence, may show repetitive behavior, may show atypical patterns of behavior and communication, may have a lack of desire to form relationships and/or may have problems with friendships or romantic relationships. In particular, reduced social competence, difficulties with social communication and repetitive behavior are a hallmark of ASD. Furthermore, a repetitive behavior (i.e. related to grooming in flies), such as inter alia hand washing or cleaning, are also observed in humans suffering from OCD.

As illustrated in the appended Examples, Cyfip^{85.1}/+ flies, which have been shown in the context of the present invention to have reduced GABA in synaptic vesicles due to an increased activity of Aralar, showed reduced social interactions in a food competition assay (i.e. reduced social competence), reduced courtship behavior (i.e. a lack of desire to form relationships), increased grooming behavior (i.e. repetitive behavior), and altered social space behavior (i.e. difficulties with social communication and/or atypical patterns of behavior). Furthermore, Cyfip1^{+/-} mice showed a reduced preference for another mouse over an object in the three-chamber test and thus, a reduced social preference/sociability.

Thus, particularly surprising, the inventors not only established a direct link between altered GABA signaling and deficits in behavior, but they also found a mechanism through which the GABA levels are altered. As illustrated in Examples 7 and 9, the GABA levels accumulate in the mitochondria of Cyfip^{85.1}/+ flies due to Aralar which the inventors have identified as mitochondrial GABA-transporter. Moreover, inhibition of Aralar normalized the just mentioned behavioral deficits of Cyfip^{85.1}/+ flies (Example 10). Furthermore, it was found that inhibition of isocitrate dehydrogenase which has been established in the context of the present invention as indirect inhibitor of Aralar, also normalized the social behavior of Cyfip^{85.1}/+ flies (Example 5). Thus, the inventors have further established a direct link between altered GABA levels, in particular in the brain, and Aralar. Thus, it is plausible that the modulation of Aralar, i.e. by an Aralar-inhibitor, may be useful for treating a neurological disorder as described herein, i.e. a neurodevelopmental and/or psychiatric disorder such as inter alia ASD, SCZ or OCD. Furthermore, it is well plausible that the modulation of Aralar may be useful for treating disorders, in particular neurological disorders as described herein, in subjects with altered GABA signaling, and/or altered GABA levels, in particular in the brain, in particular in the cytosol, synaptic vesicles and/or synapses.

As illustrated in the appended Examples, the inventors have further surprisingly found that the GABA levels were reduced in neurons and the urine of Cyfip haploinsufficient mice. Moreover, Cyfip haploinsufficient mice showed similar deficits in social behavior as Cyfip haploinsufficient flies and said deficits could be rescued by augmentation of GABA signaling (i.e. by Diazepam) in flies and mice. This strongly suggests that the findings with flies, i.e. with respect to the activity of Aralar and GABA localization, may be transferable to mammals including humans. Furthermore, this suggests that Aralar modulators/neuroactive drugs which are identified/obtained by the inventive screening methods provided herein, i.e. said screening methods which assess the behavior of a non-human animal, may be useful for treating a neurological disorder as described herein. Furthermore, it has been surprisingly found by the inventors that suppression of Aralar in another ASD/FXS fly model (Fmr1 mutants) also rescued the deficits in social behavior. This further suggests that a modulator, i.e. an inhibitor, of Aralar may be used for treating a broad range of neurological disorders as provided herein, and not be limited to certain genetic mutations. However, it is evident from the data that modulation of GABA signaling, e.g. by modulation of Aralar activity and/or mitochondrial membrane potential, as described herein, is particularly useful in subjects with Cyfip1 haploinsufficiency.

The treatment of a subject, as used herein, may refer to the treatment of a human or an animal, i.e. a domestic animal or a pet such as inter alia cattle, a dog or a monkey. However, in particular, and most preferably, as used herein, the treatment of a subject refers to the treatment of a human patient. Thus, the subject to be treated, i.e. the patient, as used herein, is preferably a subject, preferably a human, suffering from a condition, in particular a neurological disorder. Thus, said subjects to be treated/patients need a therapy and/or treatment, and may be also called a subject in need. In other words, a subject suffering from a condition, in particular a neurological disorder according to the invention, may be treated by administering a modulator of Aralar according to the invention.

Preferably herein and in the context of the invention, the neurological disorder according to the invention is treated in a subject with Cyfip1 haploinsufficiency.

Further preferably herein, the neurological disorder according to the invention is treated in a subject having altered GABA levels. In other words, a subject having altered GABA levels, in particular in the brain, may be treated by administering a modulator of Aralar according to the invention.

The GABA levels in said subject may be altered in the mitochondria, the cytosol, synaptic vesicles and/or synapses, in particular in the brain, and/or in the cerebrospinal fluid, in the blood and/or in the urine of said subject. The GABA levels are considered to be altered if they significantly differ from the GABA levels in the respective location/tissue/fluid of a control group. It is well known in the art how to determine a suitable control group and how to determine a significant difference in GABA levels. For example, the subjects of a suitable control group may be matched inter alia in age and sex to the subject suspected to have altered GABA levels, but the subjects of said control group do inter alia not have a neurological disorder or behavioral deficits as described herein. It is evident that the administration of a modulator of Aralar according to the invention to a subject is particularly useful when said subject has altered GABA levels as described herein. Thus, the GABA levels are preferably measured in a subject which is treated with a modulator of Aralar according to the invention or which is a candidate for treatment with said modulator and said GABA levels are compared to a reference level which is based on a control group as described above.

It is well known in the art, how altered GABA levels, i.e. due to Aralar activity and associated with behavioral/cognitive deficits and/or a neurological disease, can be determined, in particular in humans. As illustrated in the appended Examples, sequestration of GABA levels in the mitochondria of brain cells by Aralar, i.e. in neurons, leads to decreased GABA signaling in GABAergic synapses. Thus, an increased GABA level in the mitochondria in the brain is a good indicator of altered Aralar activity but mitochondrial GABA levels in the brain of patients may be rather difficult to measure. A decreased GABA level in the cytosol/synaptic vesicles of brain cells, i.e. neurons, is also a suitable indicator of Aralar activity as shown in the appended Examples and is a particularly useful indicator of the GABA signaling activity (which in turn regulates the behavior).

Methods for determining the cytosolic GABA levels in the brain of humans are readily available, especially magnetic resonance spectroscopy (MRS; Horder (2018), Transl Psychiatry. 8(1):106; Puts (2017), Autism Res. 10(4):608-619). It is well known that MRS is a non-invasive imaging technique which may be applied to patients. Furthermore, reference is made to Cousijn (2014) PNAS. 111(25):9301-6 which shows that the GABA signal in the brain arises mostly or almost entirely from the large cytoplasmic/cytosolic GABA pool in GABAergic neurons. Furthermore, GABA levels may be measured in the cerebrospinal fluid (CSF, see e.g. Orhan (2018), Mol Psychiatry. 23(5):1244-1250) and blood, plasma (blood plasma) and urine (e.g. Cohen (2002), Med Hypotheses. 59(6):757-8). Taking CSF, blood or urine samples and determining the GABA levels only requires routine laboratory methods. For example, the GABA levels in said samples may be determined inter alia using a GABA ELISA as described herein and in the appended Examples. Particularly practical is the determination of GABA levels in the urine, as this is absolutely non-invasive. Furthermore, the inventors found that GABA levels in the brain and in the urine were correlated and moreover that in Cyfip1^{+/-} mice, GABA levels were both reduced in primary neurons and in the urine compared to wild-type mice (Example 11).

Thus, herein, the GABA levels of a subject are in particular measured in the cytosol of the brain of said subject, preferably by magnetic resonance imaging, and/or in the in the urine of said subject. In particular, as a cost-effective and simple diagnostic, said GABA levels may be measured in the urine of said subject.

In preferred embodiments, the neurological disorder according to the invention is treated in a subject having altered GABA levels compared to a reference level, wherein the GABA level is measured in the cytosol of the brain and/or the urine of said subject. In other words, the GABA level may be determined in the cytosol of said subject, preferably in the brain, and/or in the urine, and the modulator of Aralar according to the invention is administered to the subject if said cytosolic GABA level and/or urine GABA level is increased or decreased compared to a reference level. The reference level of GABA, as used herein, refers to the normal GABA level in the respective location of a control group as described above. Thus, the modulator of Aralar according to the invention is administered to a subject suffering from a neurological disease according to the invention, when the GABA level of said subject is altered, in particular as measured in the cytosol of the brain and/or the urine of said subject.

In certain embodiments, the GABA level may be determined in the mitochondria and/or cytosol of said subject, preferably in the brain, and the modulator of Aralar according to the invention is administered to the subject if said mitochondrial and/or cytosolic GABA level is increased or decreased compared to a reference level.

In particular, the modulator of Aralar according to the invention normalizes the GABA level and/or location, preferably in the brain.

The term “normalizing”, as used herein, refers to the establishment or re-establishment of the normal condition, wherein the normal condition is the respective condition of the control group as described herein. Thus, normalizing the GABA level and/or location refers to the (re-)establishment of a normal GABA level (reference level) in at least one relevant location according to the invention, preferably in a subject having altered GABA levels as described herein. In particular, said relevant location is the mitochondria, cytosol, synaptic vesicles, synapses, in particular in the brain, and/or the cerebrospinal fluid, blood, plasma and/or urine. Preferably, said location is the location wherein the GABA levels are preferably measured as described above, hence the cytosol of the brain and/or the urine, in particular the urine.

Modulation of Aralar according to the invention is particularly useful as this allows normalizing the GABA levels in different locations without necessarily altering the total GABA levels. Thus, said modulation of Aralar may be particularly useful, when an alteration of total GABA levels is undesired. Moreover, normalizing the location of GABA, and thereby the levels in different locations, may be particularly beneficial for (re-)establishing homeostasis of healthy/normal GABA metabolism and signaling.

Furthermore, the invention relates to a modulator of Aralar for use in normalizing the GABA level and/or location in a subject, preferably in the brain of said subject. Preferably, said normalization of the GABA level and/or location occurs in a patient suffering from a neurological disease according to the invention. Normalizing the GABA level and/or location in the brain refers in particular to the GABA level in the cytosol, synaptic vesicles and/or synapses of said subject. However, said GABA level may be also measured in the urine of said subject.

As further illustrated in the appended Examples, an altered GABA levels/location due to Aralar may be driven by an energy metabolism, i.e. an altered mitochondrial activity and membrane potential in the brain. Thus, the neurological disorder according to the invention may be particularly treated in a subject having an altered mitochondrial activity and/or membrane potential, in particular in the brain. The mitochondrial activity and membrane potential may be measured, for example, inter alia, with a voltage-sensitive, positron emission tomography (PET; Da Silva (2018), Sci Rep. 2018. 8(1):6216; Momcilovic (2019), Nature. 575(7782):380-384). As regards determining whether said mitochondrial activity and membrane potential is altered, a comparison with a control group may be done as described above in the context of GABA levels.

If a subject has decreased GABA levels in the cytosol, synaptic vesicles, synapses, in particular in the brain, and/or the cerebrospinal fluid, blood, plasma and/or urine, and/or said subject has an increased mitochondrial activity and membrane potential, in particular in the brain, the modulator of Aralar according to the invention, i.e. in the context of the medical/non-medical uses as provided herein, is an inhibitor of Aralar as described further below.

If a subject has increased GABA levels in the cytosol, synaptic vesicles, synapses, in particular in the brain, and/or the cerebrospinal fluid, blood, plasma and/or urine, and/or said subject has an decreased mitochondrial activity and membrane potential, in particular in the brain, the modulator of Aralar according to the invention, i.e. in the context of the medical/non-medical uses as provided herein, is an activator of Aralar as described further below.

As explained in further detail below in the context of Aralar inhibitors and activators, and as shown in the Examples, homeostasis of GABA levels is critical for normal behavior. Thus, determining the GABA levels in a subject, i.e. a patient suffering from a neurological disorder, as described herein, is, at least in certain cases, an important step in a method of treating said neurological disorder or an important prerequisite, because it allows to choose the right means for treating said patient, i.e. an inhibitor or an activator of Aralar as described herein.

In principle, the same logic applies to the treatment of a neurological disorder in a subject having altered GABA levels and/or an altered mitochondrial activity/membrane potential, as for the treatment of a neurological disorder associated with altered GABA levels and/or an altered mitochondrial activity/membrane potential as described below in the context of Aralar inhibitors and activators. For example, an inhibitor of Aralar may be used for the treatment of a neurological disorder which is associated with decreased GABA levels in the cytosol in the brain, in a subject having decreased GABA levels as measured in the urine of said subject. Said neurological disorder may be, for example, inter alia ASD and/or schizophrenia, said subject may thus be a patient which suffers from a certain form of said neurological disorder, and said inhibitor of Aralar may be a particularly useful for treating said neurological disorder in said patient. However, this example is not meant to limit the scope of the invention but is to be regarded as a particular embodiment which further highlights the relationship between the modulator of Aralar, the neurological disease and the subject which is to be treated.

Haploinsufficiency refers to a model of dominant gene action in which a single copy of the standard (most common wild-type) allele in combination with at least one mutant allele in place of the respective further standard allele(s) is insufficient to produce the standard phenotype. In diploid organisms, the presence of one standard allele and one mutant allele is also called a heterozygous genotype. Haploinsufficiency may arise from a loss-of-function mutation in the mutant allele, such that it produces little or no gene product, which is often a protein. Although single copy of the standard allele still produces the standard amount of product, the total product is insufficient to produce the standard phenotype. Such a genotype may result in a non- or sub-standard, deleterious and/or disease phenotype. Haploinsufficiency is the standard explanation for dominant deleterious alleles. Haploinsufficiency can occur through a number of ways, for example inter alia by a mutation such as an insertion, deletion and/or alteration of at least one nucleotide which may lead to a premature stop codon and/or a frame-shift of the coding sequence. A gene may be further described as haploinsufficient if a mutation in that gene causes a loss of function and if the loss-of-function phenotype is inherited in a dominant manner relative to the wild-type allele.

As already mentioned above, mutations in the Cyfip1 gene have been associated with schizophrenia and ASD, and Cyfip1 haploinsufficiency in humans, mice and flies may confer domain-specific cognitive impairments and behavioral deficits. Furthermore, Cyfip1 has a dual role in the brain, regulating local protein synthesis via binding eIF4E and controlling actin remodelling as part of the hetero-pentameric Wave Regulatory Complex (WRC). In particular, the coding sequence of Cyfip/Cyfip1 in flies, i.e. Drosophila, which is also known as “specifically Rac1-associated protein 1” (Sra-1) refers to SEQ ID NO:5. Cyfip^{85.1}/+ flies are heterozygous mutant flies, wherein one allele of Cyfip lacks two-third of the coding region as described in Schenck (2003), Neuron 38, 887-898. As further illustrated in the appended Examples, Cyfip^{85.1}/+ flies have a Cyfip1 haploinsufficiency because, although comprising one wild-type allele of Cyfip, they show deficits in behavior, and hence not the standard phenotype (as the controls).

In the context of flies, the terms Cyfip and Cyfip1, or Cyfip and Cyfip1, are used interchangeably, and refer to the orthologue of human Cyfip1. Human Cyfip1 refers to cytoplasmic FMR1 interacting protein 1 with Gene ID: 23191 (SEQ ID NO:12 and 13). In flies, there is only one CYFIP gene which is similarly related to human CYFIP1 and CYFIP2 (67% identity at the protein level) (Schenck (2001) PNAS 17; 98(15):8844-9; Schenck (2003), Neuron 38(6):887-98). The skilled person has no difficulties in identifying an orthologue of a gene/protein in different species, i.e. in mouse, for example, by interrogating well known databases such as inter alia Ensembl, Pubmed and/or Genome Browser.

Since most studies illustrated in the appended Examples have been carried out in Drosophila flies that were Cyfip haploinsufficient (Cyfip^{85.1}/+) or Cyfip1 haploinsufficient mice, it can be safely assumed that the modulation of GABA signaling, in particular, the modulation of Aralar is particularly effective in a subject with Cyfip1 haploinsufficiency. The terms “Cyfip^{85.1}/+ flies” and “Cyfip^{85.1}/+ mutant flies” are used interchangeably herein.

Thus, in preferred embodiments, the neurological disorder according to the invention is treated in a subject with Cyfip1 haploinsufficiency.

Hence, molecules which have been already used for the treatment of a neurological disorder according to the invention, but which particularly modulate GABA signaling, e.g. the activity of Aralar, the level and/or location of GABA within a cell, and/or the behavior of a non-human animal, may be particularly useful for treating said neurological disorder in a subject with altered GABA levels, altered mitochondrial activity/membrane potential and/or Cyfip1 haploinsufficiency.

In certain embodiments of the invention, the neurological disorder is treated in a subject with altered GABA levels, altered mitochondrial activity and/or membrane potential, and/or Cyfip1 haploinsufficiency, wherein the modulator of Aralar normalizes the GABA level and/or location, preferably in the brain.

Thus, the invention also relates to a modulator of gamma-Aminobutyric acid (GABA) signaling for use in treating a neurological disorder in a subject with Cyfip1 haploinsufficiency. For example, the subject with a Cyfip1 haploinsufficiency may comprise a copy number variation (CNV), i.e. a deletion, at the 15q11.2 BP1-BP2 locus.

Therefore, the invention further relates to a modulator of GABA signaling for use in treating a neurological disorder in a human subject with a copy number variation (CNV) at the 15q11.2 BP1-BP2 locus, in particular wherein said CNV is a deletion.

As illustrated in the appended Examples, subjects with a Cyfip1 haploinsufficiency may have an altered, i.e. increased, mitochondrial activity and/or membrane potential.

Thus, the invention also relates to a modulator of gamma-Aminobutyric acid (GABA) signaling for use in treating a neurological disorder in a subject having an altered mitochondrial activity and/or membrane potential. Preferably, said subject has an increased mitochondrial activity and/or membrane potential, in particular in the brain.

As regards the treatment of a neurological disorder in a human subject with a deletion at the 15q11.2 BP1-BP2 locus, or in a subject having an increased mitochondrial activity and/or membrane potential, the same applies as is described herein in the context of a modulator of gamma-Aminobutyric acid (GABA) signaling for use in treating a neurological disorder in a subject with Cyfip1 haploinsufficiency, i.e. with respect to the neurological disorder and/or the modulator of GABA signaling.

The modulator of GABA signaling according to the invention may be comprised in a pharmaceutical composition.

A modulator of GABA signaling, as used herein, may be GABA itself, a GABA analogue, a GABA receptor agonist, a positive allosteric modulator of a GABA receptor, a GABA reuptake inhibitor and/or a GABA transaminase inhibitor. Furthermore, the modulator of GABA signaling may enhance GABA release, i.e. from a pre-synaptic neuron, and/or increase the availability of GABA in the synaptic cleft.

Usually, for treating a neurological disorder in a subject with Cyfip1 haploinsufficiency and/or an increased mitochondrial activity/membrane potential, GABA signaling must be increased. Thus, the modulator of GABA signaling according to the invention enhances GABA signaling. Enhancement of GABA signaling may be achieved, for example, by increasing extracellular GABA levels within synapse, e.g. by a GABA reuptake inhibitor, by addition of GABA, a GABA agonist, and/or by agonists/positive allosteric modulators of GABA receptors. In preferred embodiments, the modulator of GABA signaling is selected from Diazepam, GABA, valproic acid and diaminobutyric acid and/or a modulator (i.e. an inhibitor) of Aralar as described herein, wherein diaminobutyric acid is preferably DL-2,4-diaminobutyric acid. In a particular embodiment, said modulator of GABA signaling is a positive modulator (agonist) of the GABA-A receptor, preferably Diazepam.

As mentioned above, the modulator of GABA signaling, as used herein and in the context of the invention, may be a modulator of Aralar, preferably an inhibitor of Aralar. In particular, a modulator of GABA signaling which is an inhibitor of Aralar, increases GABA levels in the cytosol and/or synaptic vesicles and/or decreases GABA levels in the mitochondria of a cell containing GABA and Aralar. As described herein, the Aralar activity is usually elevated when the mitochondrial polarization is increased. Thus, said modulator of GABA signaling may modulate mitochondrial polarization and Aralar activity. In particular, said modulator of GABA signaling may decrease the mitochondrial polarization, thereby inhibit Aralar activity, and thus enhance GABA signaling. Thus, said modulator of GABA signaling may indirectly modulate GABA signaling by modulating the mitochondrial respiratory capacity, the respiratory chain, and/or an enzyme of the TCA cycle, e.g. the catalytic activity of said enzyme. Preferably, the enzyme of the TCA cycle is inhibited, preferably by an inhibitor of isocitrate dehydrogenase (IDH) and/or an inhibitor of α-ketoglutarate hydroxylase, as described herein. Thus, the modulator of GABA signaling according to the invention may comprise an inhibitor of isocitrate dehydrogenase (IDH) and/or an inhibitor of α-ketoglutarate hydroxylase, preferably ML309, as described herein, e.g. in the context of Aralar modulators.

As illustrated in the appended Examples, further drug screening with Cyfip haploinsufficient flies corroborated the surprising finding that compounds which normalize the mitochondrial membrane potential can normalize or improve the social behavior of subjects with Cyfip1 haploinsufficiency by enhancing GABA signaling via Aralar inhibition.

Thus, in further preferred embodiments, the modulator of GABA signaling comprises Diazepam, GABA, valproic acid, diaminobutyric acid, Acetohexamide, Chlorpropamide, Melatonin, Trimetazidine dihydrochloride, Ciprofibrate, and/or Aripiprazole.

Thus, the invention further relates to a pharmaceutical composition comprising Acetohexamide, Chlorpropamide, Cyclosporin A, Melatonin, Trimetazidine dihydrochloride, Ciprofibrate and/or Aripiprazole for use in treating a neurological disorder in a subject, preferably in a subject with Cyfip1 haploinsufficiency.

Furthermore, the invention relates to a pharmaceutical composition comprising Acetohexamide or Cyclosporin A, for example Acetohexamide, for use in treating a neurological disorder in a subject, preferably in a subject with Cyfip1 haploinsufficiency.

Diazepam is a positive allosteric modulator of the GABA-A receptor. Valproic acid is a GABA analogue and/or a GABA transaminase inhibitor. Diaminobutyric acid (DABA) is a GABA transaminase inhibitor and/or a GABA reuptake inhibitor (Beart (1977), Neurosci Lett. 5(3-4):193-8; Leal (2004), J Neurobiol. 61(2):189-208; Leal (2002), J Neurobiol. 50(3):245-61. Acetohexamide or Chlorpropamide have been shown to indirectly increase GABA release (Amoroso (1990), Science, 16). Furthermore, Acetohexamide or Chlorpropamide decrease the mitochondrial membrane potential (Skalska (2005), Br J Pharmacol. 145(6)). Hence, according to the surprising findings described herein, Acetohexamide or Chlorpropamide may indirectly reduce Aralar activity and normalize the GABA levels in a subject with Cyfip1 haploinsufficiency.

Melatonin has been shown to modulate GABA signaling (Cheng (2012), J Pharmacol Sci. 119(2)). Furthermore, Melatonin maintains an optimal mitochondrial membrane potential. Hence, according to the surprising findings described herein, Melatonin may indirectly affect Aralar activity and the GABA levels in a subject with Cyfip1 haploinsufficiency. Trimetazidine dihydrochloride balances the mitochondrial membrane potential (Naunyn-Schmiedebe's Archives of Pharmacology, vol. 386, no. 3, pp. 205-215, 2013; Dedkova (2013), J Mol Cell Cardiol. 59). Thus, according to the surprising findings described herein, Trimetazidine dihydrochloride may indirectly affect Aralar activity and the GABA levels in a subject with Cyfip1 haploinsufficiency.

Aripiprazole decreases the mitochondrial membrane potential (Cikánková (2019), Naunyn Schmiedebergs Arch Pharmacol. 392(10)). Hence, according to the surprising findings described herein, Aripiprazole may indirectly reduce Aralar activity and normalize the GABA levels in a subject with Cyfip1 haploinsufficiency.

Ciprofibrate, which is a PPARα agonist, may up-regulate GABAergic genes (Ferguson (2014), Neuropharmacology 86). Hence, Ciprofibrate may modulate GABA signaling by modulating the transcription or translation of genes related to GABA.

It is thus evident that Acetohexamide, Chlorpropamide, Cyclosporin A, Melatonin, Trimetazidine dihydrochloride, Ciprofibrate and/or Aripiprazole are modulators of GABA signaling and/or modulators of Aralar according to the invention.

A GABA analogue is a compound which is an analogue or derivative of GABA. A GABA analogue may be, for example, inter alia Butyric acid, Valeric acid, Isovaleric acid, Isovaleramide, Valproic acid, Valpromide, Valnoctamide, 3-Hydroxybutanal, γ-hydroxybutyric acid, Aceburic acid, γ-hydroxybutyric acid lactone, γ-hydroxybutyraldehyde, γ-hydroxyvaleric acid, γ-valerolactone, γ-hydroxycrotonic acid, γ-crotonolactone, 3-hydroxycyclopent-1-enecarboxylic acid, γ-hydroxy-γ-methylpentanoic acid, β-hydroxy-GABA, β-isobutyl-GABA, Phenibut, Baclofen, Tolibut, Phaclofen, Saclofen, Arecaidine, Gabaculine, Gabapentin, Gabapentin enacarbil, Gaboxadol, Guvacine, Isoguvacine, Isonipecotic acid, Muscimol, Nipecotic acid, L-Glutamine, N-Isonicotinoyl-GABA, Picamilon, Progabide, Tolgabide, 1,4-Butanediol, 3-Methyl-GABA, AABA/homoalanine, β-aminobutyric acid, δ-aminopentanoic acid, γ-aminobutanamide, Gabazine, γ-aminopentanoic acid, dimethyl 3-phenylglutamate hydrochloride, Glutamic acid, Homotaurine, Hopantenic acid, Isovaline, Lesogaberan, N-Anisoyl-GABA, NCS-382, Piracetam, Pivagabine and/or Vigabatrin.

A GABA receptor agonist is a drug that is an agonist for one or more of the GABA receptors, for example, inter alia a Barbiturate (in high dose), Bamaluzole, GABA, Gabamide, GABOB, Gaboxadol, Ibotenic acid, Isoguvacine, Isonipecotic acid, Muscimol, Phenibut, Picamilon, Progabide, Propofol, Quisqualamine, SL 75102, Thiomuscimol, Topiramate, Zolpidem, Eszopiclone, 1,4-Butanediol, Baclofen, gamma-Butyrolactone (GBL), gamma-Hydroxybutyric acid, gamma-Hydroxyvaleric acid (GHV), gamma-Valerolactone, Lesogaberan, Tolgabide, CACA, CAMP, and/or N4-Chloroacetylcytosine arabinoside.

A positive allosteric modulator of a GABA receptor is, in particular, a positive allosteric modulator that increases the activity of the GABAA receptor protein in the vertebrate central nervous system, for example, inter alia a benzodiazepine, diazepam (Valium), alprazolam (Xanax), zolpidem (Ambien), a barbiturate, adinazolam, allopregnanolone, tetrahydrodeoxycorticosterone, and/or tetrahydroprogesterone.

A GABA reuptake inhibitor is a drug which inhibits the reuptake of GABA from the synapse into the pre-synaptic neuron or glia cells by blocking the action of GABA transporters located in the cell membrane. Specifically, the GABA transporter 1 (GAT1) is located on the presynaptic membrane and the GAT2/3 transporters are located on the membrane of the glia cells. The reuptake of GABA leads to increased extracellular concentrations of GABA and therefore an increase in GABAergic neurotransmission. A GABA reuptake inhibitor is, for example, inter alia diaminobutyric acid (DABA), CI-966, Deramciclane, Gabaculine, Guvacine, Nipecotic acid, NNC 05-2090 NNC-711, SKF-89976A, SNAP-5114 Tiagabine (Gabitril) and/or Hyperforin.

A GABA transaminase inhibitor is an enzyme inhibitor that acts upon GABA transaminase. Inhibition of GABA transaminase enzymes reduces the degradation of GABA, leading to increased neuronal GABA concentrations. A GABA transaminase inhibitor is for example, inter alia valproic acid, diaminobutyric acid (DABA), vigabatrin, phenylethylidenehydrazine, ethanolamine-O-sulfate (EOS), and L-cycloserine.

In preferred embodiments, the modulator of GABA signaling is a modulator of Aralar as described herein.

Herein, the level of a molecule, for example Aralar or GABA, refers, in particular, to the concentration of said molecule and/or the relative abundance of said molecule compared on another condition e.g. a control group as described above.

Herein and in the context of the invention, a modulator of Aralar may directly or indirectly alter the activity of Aralar, in particular for use in treating a neurological disorder as provided herein, but also in the context of the screening methods provided herein. Said alteration of the activity of Aralar may be direct. The activity of Aralar encompasses the level of the Aralar protein, in particular in the inner mitochondrial membrane, and the activity of said Aralar protein. Preferably herein, the activity of Aralar refers to the activity of the Aralar protein, in particular to its activity as mitochondrial GABA transporter.

Direct alteration of the Aralar activity may occur at the level of the Aralar gene, mRNA, and/or protein, in particular by binding of the modulator to said Aralar gene, mRNA, and/or protein. A modulator which binds to the Aralar gene (including the regulatory sequences) is for example, inter alia a transcription factor or a chromatin modifying enzyme or an agonist thereof, or a CRISPR enzyme, i.e. Cas9, or said CRISPR enzyme fused to an activator or repressor of gene expression. A modulator that binds to the Aralar mRNA, is for example, inter alia an siRNA, microRNA, an antisense RNA or an RNA binding protein. A modulator which binds to the Aralar protein is, for example, inter alia, a small molecule, an antibody, a monobody, a protein (e.g. a protease) or a peptide. In particular, said modulator which binds to the Aralar protein modulates its function as a transporter, i.e. as a GABA transporter.

In certain embodiments, the modulator of Aralar directly alters the activity of Aralar.

Indirect alteration of the Aralar activity may, for example, inter alia affect the transcription or translation of Aralar, or the activity of the Aralar protein. For example, a modulator which affects the level or activity of one or more factors involved in the transcription of Aralar such as inter alia a transcription factor or a chromatin modifying enzyme, may thus indirectly modulate the protein level of Aralar and hence modulate the GABA transport between mitochondria and cytosol. Furthermore, modulating the forces which drive the GABA transport mediated by Aralar, may thus also indirectly alter the activity of Aralar. Aralar transporters have thus far been shown to function by exchanging an aspartate on the mitochondrial matrix for a glutamate plus a proton on the cytosolic side, with the pH gradient across the inner mitochondrial membrane forming the bioenergetic driving force for solute exchange (Monne (2019), Int J Mol Sci. 20(18)). In the context of the present invention, and as illustrated in the appended Examples, is has been found that Aralar also needs a proton gradient for the import of GABA into mitochondria (FIG. 12). Thus, the chemical driving force appears to be said pH gradient which in turn is a function of fuel availability, oxidative phosphorylation and activity of the TCA cycle (citric acid cycle). As illustrated in the appended Example 5, Cyfip^{85.1}/+ flies showed increased mitochondrial activity, in particular an increase in mitochondrial respiratory capacity through the respiratory chain, an increased mitochondrial membrane potential and an upregulation of NADH-producing enzymes, specifically the NAD-dependent isocitrate dehydrogenase (IDH) and the α-ketoglutarate hydroxylase, as well as increased isocitrate, succinate and NADH levels. Furthermore, as illustrated in the appended Example 5, inhibition of isocitrate dehydrogenase by the selective inhibitor ML309 normalized the deficient social behavior of Cyfip^{85.1}/+ flies. Thus, a modulator of the mitochondrial respiratory capacity, the respiratory chain, or an enzyme of the TCA cycle (e.g. a modulator of the catalytic activity of such an enzyme) may be an indirect modulator of the Aralar activity. In particular, such a modulator may be an inhibitor of isocitrate dehydrogenase and/or an inhibitor of α-ketoglutarate hydroxylase. Preferably, in the context of the invention and as provided herein, the inhibitor of isocitrate dehydrogenase is selected from the group consisting of the following: ML309 (2-(N-[2-(benzimidazol-1-yl)acetyl]-3-fluoroanilino)-N-cyclopentyl-2-(2-methylphenyl)acetamide; Ivosidenib (Tibsovo; (2S)—N-[(1S)-1-(2-chlorophenyl)-2-[(3,3-difluorocyclobutyl)amino]-2-oxoethyl]-1-(4-cyanopyridin-2-yl)-N-(5-fluoropyridin-3-yl)-5-oxopyrrolidine-2-carboxamide; Enasidenib (Idhifa; 2-methyl-1-[[4-[6-(trifluoromethyl)pyridin-2-yl]-6-[[2-(trifluoromethyl)pyridin-4-yl]amino]-1,3,5-triazin-2-yl]amino]propan-2-ol); Vorasidenib (AG-881; 6-(6-chloropyridin-2-yl)-2-N,4-N-bis[(2R)-1,1,1-trifluoropropan-2-yl]-1,3,5-triazine-2,4-diamine); AGI-6780 (1-[5-(cyclopropylsulfamoyl)-2-thiophen-3-ylphenyl]-3-[3-(trifluoromethyl)phenyl]urea); GSK864 ((7S)-1-[(4-fluorophenyl)methyl]-3-N-(4-methoxy-3,5-dimethylphenyl)-7-methyl-5-(1H-pyrrole-2-carbonyl)-4,6-dihydropyrazolo[4,3-c]pyridine-3,7-dicarboxamide); DS-1001b (see WO2016052697A1); BAY-1436032 (3-[2-[4-(Trifluoromethoxy)anilino]-1-[(1R,5R)-3,3,5-trimethylcyclohexyl]benzimidazol-5-yl]propanoic acid; Enasidenib mesylate (methanesulfonic acid;2-methyl-1-[[4-[6-(trifluoromethyl)pyridin-2-yl]-6-[[2-(trifluoromethyl)pyridin-4-yl]amino]-1,3,5-triazin-2-yl]amino]propan-2-ol); and Olutasidenib (5-[[(1S)-1-(6-chloro-2-oxo-1H-quinolin-3-yl)ethyl]amino]-1-methyl-6-oxopyridine-2-carbonitrile).

Preferably, in the context of the invention and as provided herein, the inhibitor of α-ketoglutarate hydroxylase is selected from the group consisting of the following: AA6 ((S)-2-[(2,6-dichlorobenzoyl) amino] succinic acid); Devimistat (CPI-613; 6,8-bis(benzylsulfanyl)octanoic acid; Succinyl phosphonate (4-oxo-4-phosphonobutanoic acid); Thiamine pyrophosphate (2-[3-[(4-amino-2-methylpyrimidin-5-yl)methyl]-4-methyl-1,3-thiazol-3-ium-5-yl]ethyl phosphono hydrogen phosphate;chloride). Preferably, said IDH inhibitor is ML309.

Said isocitrate dehydrogenase or inhibitor α-ketoglutarate hydroxylase, or other small molecule inhibitors described herein, may be used in a modified format which does not substantially alter their activity, e.g. in a complex with a pharmaceutically acceptable salt or without such a salt, as a hydrated form or non-hydrated form and/or with hydrochloride or without hydrochloride.

A preferred modulator of Aralar, i.e. a modulator that indirectly modulates the activity of Aralar according to the invention, is a modulator of isocitrate dehydrogenase, in particular an inhibitor of isocitrate dehydrogenase, preferably ML309, preferably ML309 hydrochloride. Preferably, said isocitrate dehydrogenase is IDH1 or IDH2. As indicated above, ML309 (hydrochloride) refers to 2-(N-[2-(benzimidazol-1-yl)acetyl]-3-fluoroanilino)-N-cyclopentyl-2-(2-methylphenyl)acetamide;hydrochloride.

The activity and/or inhibition of isocitrate dehydrogenase or inhibitor α-ketoglutarate hydroxylase can be easily determined by methods known in the art and as illustrated herein. For example, the levels of the substrates and/or metabolites of these enzymes (e.g. isocitrate, 2-oxoglutarate or succinate) can be readily determined, e.g. as illustrated in the appended Examples and FIG. 4.

Thus, the person skilled in the art has no difficulties in verifying and/or establishing compounds as isocitrate dehydrogenase inhibitors or α-ketoglutarate hydroxylase inhibitors, which is merely a routine exercise. Furthermore, many suitable inhibitors of isocitrate dehydrogenase or α-ketoglutarate hydroxylase are already available, as described herein.

In one embodiment, the modulator of the mitochondrial respiratory capacity, the respiratory chain, or an enzyme of the TCA cycle is rotenone, i.e. at a concentration which is not toxic for humans.

Thus, in certain embodiments, the modulator of Aralar directly alters the activity of Aralar or the nucleic acid encoding said Aralar, in particular by binding thereto, and/or indirectly controls the level and/or activity of Aralar.

A modulator of a molecule, as used herein may be an inhibitor or an activator of said molecule. Said molecule is typically a nucleic acid or a protein, preferably a protein. For example, a modulator of Aralar may be an inhibitor of Aralar or an activator of Aralar. Preferably, said modulator of Aralar is an inhibitor of Aralar.

Said modulator modulates the function of said molecule. Herein, said molecule is to be understood as the entirety of all molecules of the same type, e.g. the same protein including all modifications of said protein. Thus, the function of said molecule may be modulated by altering the level of said molecule and/or the activity of said molecule. The level and/or activity of said molecule may also depend on the location of said molecule. Thus, the function of said molecule may be modulated, by altering the level, location and/or activity, e.g. inter alia the enzymatic activity, binding activity or signaling activity of said molecule.

An inhibitor of a molecule inhibits the function of said molecule, in particular said inhibitor may decrease the level of said molecule, inhibit the activity of said molecule and/or inhibit the activation of said molecule. In particular, an inhibitor of Aralar inhibits the function of Aralar. Thus, said inhibitor of Aralar may decrease the level of Aralar, inhibit the activity of Aralar and/or inhibit the activation of Aralar. In preferred embodiments, i.e. in the context of the medical/non-medical uses according to the invention, the inhibitor of Aralar inhibits the transport activity of Aralar, especially the GABA transport into mitochondria. In particular, an inhibitor of Aralar may inhibit the activity of Aralar, i.e. the Aralar-mediated GABA transport into mitochondria. Preferably, said inhibitor decreases the mitochondrial membrane potential and/or decreases the activity of the TCA cycle. In particular, said inhibitor may be an inhibitor of isocitrate dehydrogenase and/or an inhibitor of α-ketoglutarate hydroxylase, as provided herein.

Inhibition of the GABA transport activity of Aralar may be due to decreasing the mitochondrial membrane potential.

Preferably herein, i.e. in the context of the medical and non-medical uses, the modulator of GABA signaling enhances GABA signaling, and/or a modulator of Aralar is an inhibitor of Aralar as provided herein.

Thus, an enhancer of GABA signaling, e.g. an inhibitor of Aralar according to the invention, i.e. an inhibitor as provided herein and/or an inhibitor of Aralar as identified by the inventive screening methods provided herein, may be used for treating a neurological disorder as described above in the context of neurological disorders. Preferably, said neurological disorder is a neurodevelopmental disorder, a psychiatric disorder and/or a disorder that is associated with synaptic dysfunction, wherein said neurological disorder is accompanied by behavioral deficits. Preferably, said behavioral deficits comprise at least one, preferably at least 2, preferably at least 4, preferably at least 8 of the group consisting of: reduced social competence; atypical patterns of behavior and communication; poor nonverbal communication skills such as lack of eye contact and meaningful gestures and facial expressions; repetitive or rigid language and/or strange or poor speech; a decreased ability to understand reality; reduced emotional expression; lack of motivation; impaired theory of mind; impaired memory; and/or impaired learning. More preferably, said deficits comprise (i) reduced social competence and/or (ii) repetitive behavior.

In one embodiment, an enhancer of GABA signaling, e.g. an inhibitor of Aralar according to the invention, is used for treating a neurodevelopmental disorder and/or a psychiatric disorder that is associated with a synaptic dysfunction. Preferably, said synaptic dysfunction involves a GABAergic synapse, preferably in the brain.

In a preferred embodiment, enhancer of GABA signaling, e.g. an inhibitor of Aralar according to the invention, is used for treating a neurodevelopmental disorder and/or psychiatric disorder which is accompanied by said at least one, preferably at least 2, preferably at least 4, preferably at least 8 behavioral deficits, preferably (i) reduced social competence and/or (ii) repetitive behavior, wherein said disorder is further associated with said synaptic dysfunction. In particular, said neurodevelopmental disorder and/or psychiatric disorder which is accompanied by said behavioral deficits and associated with said synaptic dysfunction, is ASD, SCZ, FXS and/or a disability featuring synaptic dysfunctions, preferably wherein the disability featuring synaptic dysfunctions is Schizophrenia, Epilepsy, Down Syndrome, Angelman syndrome, Alzheimer's disease and/or Parkinson's disease.

In a very preferred embodiment, enhancer of GABA signaling, e.g. an inhibitor of Aralar according to the invention, is used for treating autism spectrum disorder, schizophrenia and/or obsessive-compulsive disorder, in particular autism spectrum disorder. Preferably, said autism spectrum disorder is fragile X syndrome or is associated with fragile X syndrome.

An activator of a molecule enhances the function of said molecule, in particular said activator may increase the level of said molecule, enhance the activity of said molecule and/or promote the activation of said molecule. Of note, enhancing the activity of an inactive molecule is to be understood as at least activating said molecule. In particular, an activator of Aralar enhances the function of Aralar. Thus, said activator of Aralar may increase the level of Aralar, enhance the activity of Aralar and/or promote the activation of Aralar. In particular, the activator of Aralar may activate and/or enhance the transport activity of Aralar, especially the GABA transport into mitochondria.

Since the level, location and/or activity of the molecule which is to be modulated can be determined as described herein, the inhibitory or activating action of a modulator of said molecule can be also readily determined, for example by comparing to a negative control which does not comprise said modulator. Suitable assays for determining the activity of Aralar are described in the appended Examples, and further below in the context of the screening methods according to the invention. Furthermore, assays for determining the mitochondrial respiratory capacity, the activity of the respiratory chain, or the activity of an enzyme of the TCA cycle are described in the appended Examples. However, determining the level, location and/or activity of a molecule, e.g. of Aralar, GABA, or a protein or metabolite involved in a mitochondrial process, is not limited to the assays described herein, but the skilled person is well aware of further suitable assays.

In the context of the invention, the function of Aralar which is modulated, is preferably at least the transport of GABA. In particular, the GABA transport into mitochondria can be determined by measuring GABA levels in the mitochondria and/or the cytosol.

In particular, an inhibitor of Aralar decreases the mitochondrial GABA level and/or increases the cytosolic GABA level, i.e. relative to a control. Preferably, said inhibitor increases the ratio of the cytosolic GABA level over the mitochondrial GABA level.

In particular, an activator of Aralar increases the mitochondrial GABA level and/or decreases the cytosolic GABA level, i.e. relative to a control. Preferably, said activator decreases the ratio of the cytosolic GABA level over the mitochondrial GABA level.

In the context of the invention, and as illustrated in the appended Examples (i.e. FIG. 7c), the cytosolic GABA level is preferably the GABA level in cellular vesicles, in particular in synaptic vesicles.

The inhibitor of Aralar may be selected from at least one of the groups consisting of (i) a small molecule drug; (ii) a peptide; (iii) a nucleic acid molecule inhibiting Aralar translation, transcription, expression and/or activity; (iv) an antibody specifically binding to Aralar; and (v) a protein/peptide promoting the degradation of Aralar. Said antibody specifically binding to Aralar may be also a nanobody (single-domain antibody). In preferred embodiments, the inhibitor of Aralar is a small molecule drug. In certain embodiments, said inhibitor of (i) or said small molecule drug is at least one selected from the group consisting of pyridoxal 5′-phosphate, bathophenanthroline, mersalyl, chebulinic acid, suramin, HgCl₂, p-chloromercuriphenylsulfonate, and diethyl pyrocarbonate. Reference is made to Amoedo (2016), Biochim Biophys Acta 1863, 2394-2412, and Palmieri (2001), EMBO J. 20(18):5060-9). In certain embodiments, said inhibitor of (i) or said small molecule drug is at least one selected from the group consisting of pyridoxal 5′-phosphate, bathophenanthroline, mersalyl, chebulinic acid and suramin. In other preferred embodiments, said inhibitor of (i) or said small molecule drug is at least one selected from the group consisting of bathophenanthroline, mersalyl, chebulinic acid and suramin. In certain embodiments, said inhibitor of (i) or said small molecule drug is at least one selected from the group consisting of ML309, Ivosidenib, Enasidenib, AGI-6780, GSK864, DS-1001b, BAY-1436032, Enasidenib mesylate and Olutasidenib, preferably ML309. In certain embodiments, said inhibitor of (i) or said small molecule drug is at least one selected from the group consisting of AA6, Devimistat, Succinyl phosphonate and Thiamine pyrophosphate.

In one embodiment of the invention, the modulator of Aralar for use in treating a neurological disorder according to the invention is an inhibitor of Aralar as provided herein, wherein said inhibitor is pyridoxal 5′-phosphate.

In one embodiment of the invention, the modulator of Aralar for use in treating a neurological disorder according to the invention is an inhibitor of Aralar, wherein said inhibitor is bathophenanthroline.

In one embodiment of the invention, the modulator of Aralar for use in treating a neurological disorder according to the invention is an inhibitor of Aralar as provided herein, wherein said inhibitor is mersalyl.

In one embodiment of the invention, the modulator of Aralar for use in treating a neurological disorder according to the invention is an inhibitor of Aralar as provided herein, wherein said inhibitor is chebulinic acid.

In one embodiment of the invention, the modulator of Aralar for use in treating a neurological disorder according to the invention is an inhibitor of Aralar as provided herein, wherein said inhibitor is suramin.

In particular, pyridoxal 5′-phosphate, bathophenanthroline, mersalyl, chebulinic acid, suramin, HgCl₂, p-chloromercuriphenylsulfonate, and diethyl pyrocarbonate refer to the following compounds:
1) Pyridoxal phosphate (Molecular Formula: C8H10NO6P; IUPAC Name: 4-formyl-5-hydroxy-6-methylpyridin-3 yl) methyl dihydrogen phosphate; CAS Number: 853645-22-4);
2) Bathophenanthroline (Molecular Formula: C24H16N2; IUPAC Name: 4,7-diphenyl-1,10-phenanthroline; CAS Number: 1662-01-7);
3) Mersalyl (Molecular Formula: C13H17HgNNaO6; IUPAC Name: sodium;[3-[[2-(carboxylatomethoxy)benzoyl]amino]-2-methoxypropyl]mercury;hydrate; CAS Number: 492-18-2);
4) Chebulinic acid (Molecular Formula: C41H32O27; IUPAC Name: 2-[(4R,5S,7R,8R,11S,12S,13S,21S)-13,17,18-trihydroxy-2,10,14-trioxo-5,21-bis[(3,4,5-trihydroxybenzoyl)oxy]-7-[(3,4,5-trihydroxybenzoyl)oxymethyl]-3,6,9,15-tetraoxatetracyclo[10.7.1.14,8.016,20]henicosa-1(19),16(20),17-trien-11-yl]acetic acid; CAS Number: 18942-26-2);
5) Suramin (Molecular Formula: C51H40N6O23S6; IUPAC Name: 8-[[4-methyl-3-[[3-[[3-[[2-methyl-5-[(4,6,8-trisulfonaphthalen-1yl)carbamoyl]phenyl]carbamoyl]phenyl]carbamoylamino]benzoyl]amino]benzoyl]amino]naphthalene-1,3,5-trisulfonic acid; CAS Number: 145-63-1);
6) HgCl₂ (IUPAC Name: dichloromercury; CAS Number: 7487-94-7)
7) p-chloromercuriphenylsulfonate (Molecular Formula: C6H5ClHgO3S; IUPAC Name: chloro-(4-sulfophenyl)mercury; CAS Number: 554-77-8);
8) Diethyl pyrocarbonate (Molecular Formula: C6H1005; IUPAC Name: ethoxycarbonyl ethyl carbonate; CAS Number: 1609-47-8).

Said compounds (1-8) also refer to the following chemical structures:

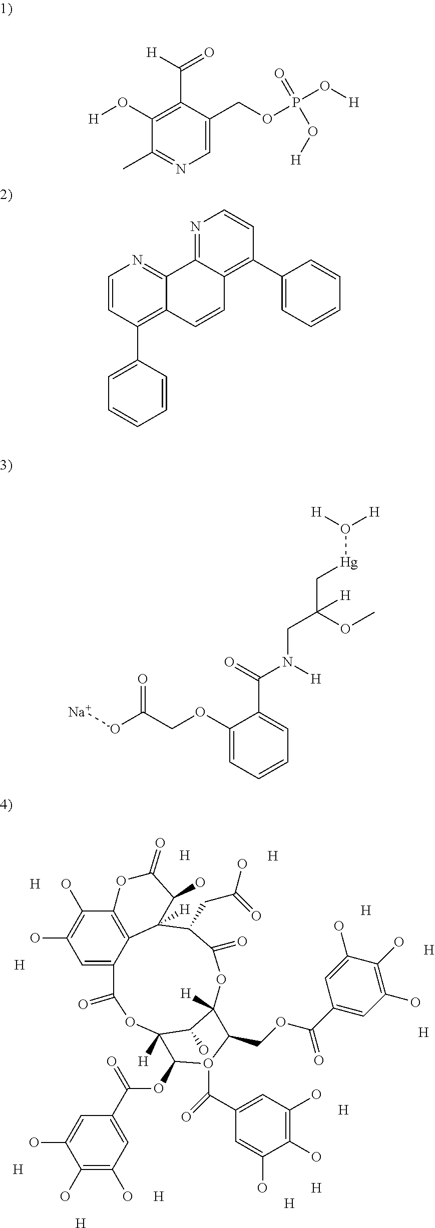

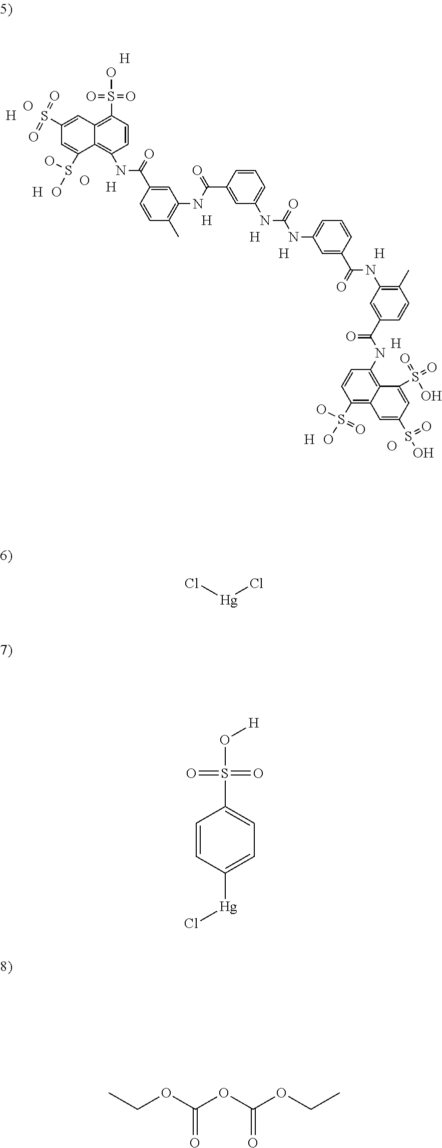

In certain embodiments, said inhibitor of (i) or said small molecule drug comprises at least one aryl group.

In preferred embodiments of the invention, the enhancer of GABA signaling, e.g. the modulator of Aralar, for use in treating a neurological disorder according to the invention is an inhibitor of Aralar as provided herein, wherein said inhibitor is a modulator of the mitochondrial respiratory capacity, the respiratory chain, or an enzyme of the TCA cycle, and wherein said inhibitor decreases the mitochondrial membrane potential, and preferably wherein said inhibitor is not toxic to humans at an effective dose. Said modulator of the mitochondrial respiratory capacity, the respiratory chain, or an enzyme of the TCA cycle may be an inhibitor of isocitrate dehydrogenase and/or an inhibitor of α-ketoglutarate hydroxylase as provided herein. In particular, said IDH inhibitor reduces the activity of the TCA cycle and/or the proton gradient over the inner mitochondrial membrane which reduces the activity of Aralar. Preferably, said inhibitor is an inhibitor of isocitrate dehydrogenase, preferably ML309. In particular, said isocitrate dehydrogenase may be IDH2 or IDH3 (IDH3A/B). Human IDH2 refers to NCBI Entrez Gene ID 3418; Human IDH3A refers to NCBI Entrez Gene ID 3419; and Human IDH3B refers to NCBI Entrez Gene ID 3420.

In a very preferred embodiment of the invention, the modulator of Aralar for use in treating a neurological disorder according to the invention is an inhibitor of Aralar as provided herein, wherein said inhibitor is an inhibitor of isocitrate dehydrogenase, preferably ML309.

Preferably, said inhibitor of option (ii) of the inhibitor of Aralar described above and/or said peptide, binds to Aralar and thereby blocks the GABA transporting activity of said Aralar.

Preferably, said inhibitor of option iii) of the inhibitor of Aralar described above and/or said nucleic acid molecule inhibiting Aralar translation, transcription, expression and/or activity is an antisense molecule, i.e. an antisense RNA, or an interfering RNA, i.e. a siRNA. In particular, said nucleic acid molecule comprises 10 to 50, preferably 10 to 30, preferably 15 to 25, preferably 18 to 22 nucleotides as consecutive nucleotide sequence, wherein said nucleotide sequence is complementary to a nucleotide sequence comprised in the Aralar mRNA. The term “complementary” means that said nucleotide sequence binds strongly enough to the Aralar mRNA that said Aralar mRNA is degraded and/or the translation thereof is suppressed. Typically, at least 90%, preferably at least 95%, preferably all of the nucleotide sequence of said nucleic acid molecule are complementary to said nucleotide sequence comprised in the Aralar mRNA. In particular, said Aralar mRNA comprises SEQ ID NO:1 or SEQ ID NO:3 with the difference that the thymidines (“T”) are uridines (“U”).

Preferably, said inhibitor of option v) of the inhibitor of Aralar described above and/or said protein/peptide is a protease which promotes the degradation of Aralar, and/or a protein which marks Aralar for degradation. Preferably, said protease is specific for Aralar. In particular, said Aralar-specific protease does not promote the degradation of other proteins in a cell, i.e. a brain cell and/or neuron.

In particular, a suitable inhibitor of Aralar according to the invention is the small molecule drug pyridoxal 5′-phosphate, preferably pyridoxal 5′-phosphate hydrate and/or the IDH inhibitor ML309.

Pyridoxal 5′-phosphate (PLP) is a vitamin B6 phosphate, in particular the active form of vitamin B6 serving as a coenzyme for the synthesis of inter alia amino acids and neurotransmitters.

In certain embodiments, the modulator of Aralar according to the invention is an inhibitor of Aralar, wherein said inhibitor increases GABA levels in the cytosol/synaptic vesicles and/or decreases GABA levels in the mitochondria of a cell containing GABA and Aralar.

In certain embodiments, the modulator of Aralar according to the invention is an inhibitor of Aralar, wherein said inhibitor is used for treating the neurological disorder in a subject having decreased GABA levels and/or an increased mitochondrial activity/membrane potential.

As illustrated in the appended Examples, the activity of Aralar to transport GABA into the mitochondria can be increased by increasing the mitochondrial membrane potential with oligomycin.

Thus, in certain embodiments, i.e. in the context of the medical and/or non-medical uses according to the invention, an activator of Aralar increases the mitochondrial membrane potential. In particular, said Aralar-activator may be an inhibitor of complex V-ATP synthase, preferably oligomycin.

Furthermore, an activator of Aralar may be, for example inter alia a CRISPR enzyme, i.e. Cas9, zinc-finger transcription factor (ZFN) or a transcription activator-like effector (TALE) which is fused to an activator of gene expression, such as inter alia VP64 or VP64-p65-Rta. In particular, said CRISPR enzyme, ZNF or TALE binds to the enhancer and/or promoter of the Aralar gene and/or to the enhancer and/or promoter of at least one transcription factor which enhances the transcription of Aralar. Preferably, said CRISPR enzyme, ZNF or TALE binds to the enhancer and/or promoter of the Aralar gene.

As illustrated in the Examples, the homeostasis of GABA levels in the brain, which is in particular regulated by the activity of Aralar, is crucial for normal behavior. Whereas in Cyfip^{85.1}/+ mutant flies, inhibition of Aralar (Example 10), reduction of Aralar levels (Example 9), elevation of GABA levels (Example 8) or impairment of the TCA cycle (Example 5, i.e. the reduction of IDH levels) thereby indirectly reducing the activity of Aralar and enhancing GABA signaling, normalized the behavior of said flies, the effects were opposite in wild-type flies. For example, treatment of wild type flies with oligomycin (inhibitor of complex V-ATP synthase) which indirectly increases Aralar activity by increasing the mitochondrial membrane potential mimicked behavioral deficits of the Cyfip^{85.1}/+ mutant flies. Moreover, in wild-type flies, the same reduction of Aralar levels (Example 9), elevation of GABA levels (Example 8) or impairment of the TCA cycle (Example 5) caused behavioral deficits in flies which were similar to Cyfip^{85.1}/+ mutant flies. In other words, the same intervention or medication may be beneficial for one patient group and harmful for another patient group. These data further demonstrate that an enhancer of GABA signaling, e.g. an inhibitor of Aralar, according to the invention, is particularly useful for treating a neurological disorder as described herein (i.e. a neurodevelopmental and/or psychiatric disorder such as ASD or schizophrenia), in a subject with Cyfip1 haploinsufficiency.

Furthermore, inhibition of Aralar may be particularly useful for the treatment of those neurological disorders described herein which are associated with decreased GABA levels in synaptic vesicles, synapses, cerebrospinal fluid, blood, plasma and/or urine, and/or an increased mitochondrial activity and/or mitochondrial membrane potential, in particular in the brain. Autism spectrum disorder and schizophrenia are particularly prominent examples of this kind of neurological disorders, but it is well plausible that inhibition of Aralar is also useful for further neurological disorders which are associated with decreased GABA levels in synaptic vesicles, synapses, cerebrospinal fluid, blood, plasma and/or urine, and/or an increased mitochondrial activity and/or mitochondrial membrane potential.

Thus, in certain embodiments of the invention, the modulator of Aralar for use in treating a neurological disorder is an inhibitor of Aralar as described herein and said neurological disorder is associated with decreased GABA levels in synaptic vesicles, synapses, cerebrospinal fluid, blood, plasma and/or urine, and/or an increased mitochondrial activity and/or mitochondrial membrane potential, in particular in the brain.

In contrast, inhibition of GABA signaling, e.g. activation of Aralar, may be particularly useful for the treatment of those neurological disorders described herein which are associated with increased GABA levels in the cytoplasm, synaptic vesicles, synapses, cerebrospinal fluid, blood, plasma and/or urine, and/or a decreased mitochondrial activity and/or mitochondrial membrane potential, in particular in the brain. In particular, said neurological disorder (which is associated with increased GABA levels in the cytoplasm) may be GABA-transaminase deficiency or succinic semialdehyde dehydrogenase (SSADH) deficiency (Malaspina (2016), Neurochem Int. 99:72-84).

Thus, in certain embodiments of the invention, the modulator of Aralar for use in treating a neurological disorder is an activator of Aralar as described herein and said neurological disorder is associated with increased GABA levels in synaptic vesicles, synapses, cerebrospinal fluid, blood, plasma and/or urine, and/or a decreased mitochondrial activity and/or mitochondrial membrane potential, in particular in the brain.

Cytosolic GABA levels in the brain, in particular in synaptic vesicles, may be determined as described above, for example, inter alia by simple magnetic resonance spectrometry (MRS) or two-dimensional protons MRS (Horder (2018), Transl Psychiatry. 8(1):106; Puts (2017), Autism Res. 10(4):608-619). It is also well known in the art how to determine the GABA concentration in cerebrospinal fluid (e.g. Orhan (2018), Mol Psychiatry. 23(5):1244-1250) and blood, urine or plasma (e.g. Cohen (2002), Med Hypotheses. 59(6):757-8). The mitochondrial activity and membrane potential may be measured, for example, inter alia, with a voltage-sensitive, positron emission tomography (PET; Da Silva (2018), Sci Rep. 2018. 8(1):6216; Momcilovic (2019), Nature. 575(7782):380-384).

The invention further relates to a method for normalizing the GABA level and/or location as described herein in a subject comprising administering to said subject a modulator of Aralar. Preferably, said subject suffers from a neurological disorder, preferably a neurodevelopmental disorder as described herein. In principle, the same applies as is described herein in the context of a modulator of Aralar for use in normalizing the GABA level and/or location in a subject, preferably in the brain of said subject.

Furthermore, the invention relates to a method for treating a neurological disorder, preferably a neurodevelopmental disorder in a subject, comprising administering to said subject a modulator of Aralar. In principle, the same applies as is described herein in the context of a modulator of Aralar for use in treating a neurological disorder.

The invention also relates to a method for treating a neurological disorder in a subject with Cyfip1 haploinsufficiency, comprising administering to said subject a modulator of GABA signaling as described herein. Preferably, said modulator is a modulator of Aralar. In principle, the same applies as is described herein in the context of a modulator of gamma-Aminobutyric acid (GABA) signaling for use in treating a neurological disorder in a subject with Cyfip1 haploinsufficiency.

Furthermore, the invention relates to a method for treating a neurological disorder in a subject having an altered, preferably increased, mitochondrial activity and/or membrane potential, comprising administering to said subject a modulator of GABA signaling as described herein. Preferably, said modulator is a modulator of Aralar. In principle, the same applies as is described herein in the context of a modulator of gamma-Aminobutyric acid (GABA) signaling for use in treating a neurological disorder in a subject having an altered, preferably increased, mitochondrial activity and/or membrane potential.

In one aspect, the invention relates to a modulator of Aralar as provided herein for use as a medicament. In particular, said modulator of Aralar is an inhibitor of Aralar.

Thus, in a further aspect, the invention relates to a pharmaceutical composition comprising a modulator of Aralar as provided herein. In particular, said modulator of Aralar is an inhibitor of Aralar.

A modulator of Aralar according to the invention may be also used for non-therapeutic/non-medical purposes, in particular for preventing, alleviating, reversing, and/or stopping at least one deficit in behavior as described above in the context of neurological disorders.

In particular, when used for a non-medical purpose, the modulator of Aralar is administered to or taken in by a subject that is not suffering from a neurological disorder, i.e. not from a neurodevelopmental and/or psychiatric disorder, as described herein. Said subject may have certain traits of a subject suffering from such a neurological disorder but those traits do not form a proper clinical picture/are not pathologic, i.e. the there are less of such traits and/or the traits are milder. For example, a human which is not suffering from a psychiatric disorder may have difficulties in creating connections with other people and desire improving its social competence, or he/she may have difficulties with reading, writing, working memory and/or mathematics.

Thus, the invention further relates to the non-medical use of a modulator of Aralar for preventing, alleviating, reversing, and/or stopping at least one, preferably at least 2, preferably at least 3, preferably at least 6, preferably at least 10, behavioral deficit(s).

In particular, said at least 10 deficits comprise difficulties creating connections with other people, problems with friendships or romantic relationships, reduced social competence, lack of social awareness, unfocused thinking, reduced social engagement, lack of motivation, reduced attention, impaired learning, and impaired memory. In particular, said at least 6 deficits comprise difficulties creating connections with other people, reduced social competence, unfocused thinking, lack of motivation, impaired learning, and impaired memory, e.g. impaired working memory. In particular, said at least 3 deficits comprise reduced social competence, lack of motivation, and impaired memory. In particular, said at least 2 deficits comprise (i) reduced social competence and (ii) lack of motivation. In particular, said at least one deficit comprises reduced social competence. Said reduced social competence comprises inter alia difficulties creating connections with other people, problems with friendships or romantic relationships, lack of social awareness, reduced social engagement and/or a lack of motivation. Furthermore, said deficits may comprise deficits in reading, writing and/or mathematics such as deficits in grammatical and/or mathematical reasoning and/or working memory.

Furthermore, the invention also relates to the non-medical use of a modulator of gamma-Aminobutyric acid (GABA) signaling for preventing, alleviating, reversing, and/or stopping at least one behavioral deficit in a subject with Cyfip1 haploinsufficiency, as just described above in the context of the non-medical use of an Aralar modulator. In particular, said subject is not suffering from a neurological disorder; see also Woo (2019), Biol Psychiatry. 2019 Aug. 15; 86(4):306-314).

Said modulator of GABA signaling may comprise Diazepam, GABA, valproic acid, diaminobutyric acid, Acetohexamide, Chlorpropamide, Melatonin, Trimetazidine dihydrochloride, Ciprofibrate, Aripiprazole, an inhibitor of isocitrate dehydrogenase (IDH) such as ML309, and/or an inhibitor of α-ketoglutarate hydroxylase.

The modulator of GABA signaling, e.g. a modulator of Aralar, for a non-medical use described above is preferably an enhancer of GABA signaling, e.g. an inhibitor of said Aralar, according to the invention. The administration and/or dose of said modulator for a non-medical use may differ from it use in a method of treatment. In particular, the dose of said modulator may be lower for a non-medical use as compared to the use in a method of treatment.

Said modulator of GABA signaling, e.g. a modulator of Aralar, may be comprised in a food product, a food for special medical purposes and/or a medical food. Furthermore, said modulator of GABA signaling, e.g. a modulator of Aralar, may be administered to a subject through a nasal drug delivery device.

A food for special medical purposes, as used herein, refers to food products for the dietary management (under medical supervision), of individuals who suffer from certain diseases, disorders or medical conditions. These foods are intended for the exclusive or partial feeding of people whose nutritional requirements cannot be met by normal foods. A medical food, as used herein, refers to a food which is formulated to be consumed or through enteral administration under the supervision of a physician and which is intended for the specific dietary management of a disease or condition for which distinctive nutritional requirements, based on recognized scientific principles, are established by medical evaluation. A food product refers to any substance consumed to provide nutritional support for an organism. A food product may be of biological origin, and contain essential nutrients, such as carbohydrates, fats, proteins, vitamins, or minerals and/or consist essentially of an aqueous solution. A food product is ingested by a subject and assimilated by the subject's cells to provide energy, maintain life, or stimulate growth. A food product can be taken in by a subject or administered to a subject. Preferably, a food product is eaten or drunk.

It is further contemplated in the context of the present invention, that a modulator of GABA signaling, e.g. a modulator of Aralar, may be used for controlling pests, i.e. insects, in the environment or fields (but typically not in human or animal bodies) by altering the behavior of said pests, in particular by reducing the social behavior and/or mating behavior of said pests. As described herein, homeostasis of GABA signaling is important for normal behavior. Thus, the application of an inhibitor or activator of Aralar as provided herein may disturb the behavior of pests. In particular, disturbance of the mating/courtship behavior may be useful for decimate pest populations in the environment, such as inter alia populations of mosquitoes which transmit diseases such as inter alia malaria or dengue fever.

As described above, modulators, i.e. inhibitors and activators, or Aralar are well known in the art. Moreover, the skilled person can readily determine whether a molecule is a modulator, i.e. an inhibitor or activator, of Aralar.

A suitable way to determine whether a molecule is a modulator of Aralar is determining the mitochondrial and/or cytosolic GABA levels within a cell, preferably within a cell of the neural lineage, preferably a neuron, preferably a GABAergic neuron. Said cell may be any cell containing GABA and Aralar. This means that GABA and Aralar molecules must be present in said cell, preferably at a level which is comparable to a cell of the neural lineage, preferably a neuron, preferably a GABAergic neuron. Because Aralar must be present at the protein level, this may be verified by determining Aralar expression at the protein level in said cell. However, since Aralar expression at the protein level requires expression of Aralar at the mRNA level, the presence of Aralar may be also determined at the mRNA level/level of gene expression. Said GABA and Aralar may be introduced into a cell artificially, for example by injection or lipofection. Said cell may have been genetically modified to express Aralar and the enzymes required for synthesis of GABA, i.e. the enzyme glutamate decarboxylase (GAD). Preferably, the cell for determining GABA levels/location naturally contains Aralar and GABA. Said cell containing Aralar and GABA may further have an insertion, deletion, premature stop codon, and/or frame-shift mutation within the Cyfip1 gene and or is derived from a subject with Cyfip1 haploinsufficiency and/or a subject with impaired behavior. As regards the behavior, the same applies as is described below in the context of normalizing the behavior of the non-human animal. Preferably, the cell for determining GABA levels/location is maintained in vitro or ex vivo in culture. The terms “in vitro” and “ex vivo” both refer to culturing cells outside of a living organism, i.e. in a culture dish/well. Furthermore, “in vitro” rather refers to the culturing of cell lines, whereas “ex vivo” rather refers to culturing of primary cells, i.e. cells/tissues which have been freshly obtained from a subject. Preferably, the cell for determining GABA levels/location, i.e. when cultured in vitro, is a human cell, a mouse cell or an insect cell, preferably a human cell. It is well known in the art, how to culture suitable cells, in particular neurons, for determining GABA levels/location. For example, a method for culturing GABAergic neurons is described in Turko (2019) J Vis Exp. 148. Said referenced document is incorporated in its entirety herein.

Furthermore, it is well known, how to measure GABA levels at subcellular resolution, i.e. to distinguish mitochondria and the cytosol or synaptic vesicles. For example, inter alia immunochemistry (immunostaining) can be employed, in particular by simultaneously staining GABA, a mitochondrial marker, and a marker for synaptic vesicles. Suitable mitochondrial markers are, for example, inter alia VDAC/Porin, ATP5B and TOM20. Suitable markers for synaptic vesicles are, for example, inter alia synaptophysin and synaptotagmin. Mitochondrial markers or synaptic vesicles may be also introduced into the cells by means of vectors expressing detectable fusion-proteins locating to either mitochondria or synaptic vesicles (Newman (2016), Cell Logist. 6(4):e1247939). Detectable fusion-proteins may comprise fluorescent proteins such as inter alia GFP or mCherry. Furthermore, small molecules are readily available to specifically stain mitochondria, e.g. inter alia mitotrackers, for example, inter alia 1H,5H,11H,15H-Xantheno[2,3,4-ij:5,6,7-i′j′]diquinolizin-18-ium, 9-[4-(chloromethyl)phenyl]-2,3,6,7,12,13,16,17-octahydro-, chloride (also known as Invitrogen™ MitoTracker™ Red CMXRos). Furthermore, FM-dyes/styryl dyes may be used for staining synaptic vesicles (Lazarenko (2018), Bio Protoc. 8(2)). Specific antibodies for staining GABA are also well known in the art, for example, inter alia rabbit polyclonal anti-GABA antibody Cat #A2052 from Sigma-Aldrich/Merck which detects at least fly, mouse and human GABA. Specifically, bound GABA antibodies may be detected, for example, by use of fluorescent dyes or a chromogenic enzyme such as inter alia horse radish peroxidase or alkaline phosphatase, either when those dyes or enzymes are conjugated to said GABA antibodies or by using a secondary antibody (conjugated to such a dye or enzyme) directed against the GABA antibodies. Furthermore, genetically encoded GABA sensors have been developed which may be used for determining GABA levels/localization (i.e. in mitochondria) in living cells (Marvin (2019) Nat Methods, 16(8):763-770). Microscopy methods and image quantification tools (e.g. comprising inter alia an image background subtraction method) are readily available in the art to quantify GABA levels in the mitochondria and the cytosol, i.e. the synaptic vesicles.

Also, a suitable way to determine whether a molecule is a modulator of Aralar is determining the mitochondrial and/or cytosolic GABA levels in the brain of a non-human animal, preferably a fly, preferably Drosophila. For example, as also illustrated in the appended Examples, the (potential) modulator of Aralar may be mixed with the food with which said non-human animal is fed. An immunochemistry assay as described above, and/or as illustrated in the appended Example 7, may be used for determining the mitochondrial and/or cytosolic GABA levels in the brain, in particular in sections of the brain. Furthermore, GABA levels in either mitochondria or the cytosol/synaptic vesicles may be measured by first fractionating the cell material, for example, inter alia the brain of said non-human animal or the cultured cells as described above, to enrich for mitochondria, cytosol, synaptic vesicles, or cytosol/synaptic vesicles, e.g. as described in Depner (2014), Nature protocols 9, 2796-2808, and then perform a GABA ELISA Enzyme immunoassay, e.g. as described in GABA ELISA, REF ID59301, IBL international, 2012-01-20), and/or as illustrated in the appended Example 7. Said referenced documents are incorporated in their entirety herein. Furthermore, the GABA levels may be measured in the fractionated cell material by HPLC-MS. A suitable protocol for performing HPLC-MS (HILIC-MS/MS; HILIC-HRMS), i.e. on fly brain extracts, is described in the appended Examples (Example 7).

In brief, the person skilled in the art has a variety of well-known assays at hand and has no difficulties in determining GABA levels separately in mitochondria and the cytosol or synaptic vesicles of cells, in particular in brain cells and/or cells of the neural lineage/neurons. Thus, the skilled person can easily determine the ratio of the cytosolic GABA level over the mitochondrial GABA level. As illustrated in the appended Example 9, a reduction of the Aralar level—which is similar to the inhibition of Aralar and/or an overall reduced Aralar activity—leads to an increased ratio of the cytosolic GABA level over the mitochondrial GABA level. Thus, determining the ratio of the cytosolic GABA level over the mitochondrial GABA level is a highly suitable way to determine the activity of Aralar. Hence, the person skilled in the art has no difficulties in determining whether a certain molecule/drug/compound/composition/pharmaceutical is a modulator of Aralar, i.e. if it is an inhibitor or an activator of said Aralar.

A cell of the neural lineage, as used herein, refers to a neuron or any cell which has the capacity to develop into a neuron but does not have the capacity, at least without forced transdifferentiation (e.g. by genetic manipulation), to develop into a cell of another lineage, i.e. the endodermal or mesodermal cell lineage. Preferably, said cell of the neural lineage comprises synaptic vesicles and/or a neurite, preferably an axon. Preferably, said cell of the neural lineage comprising synaptic vesicles and/or a neurite is a neuron.

A neuron, also called a nerve cell, is an electrically excitable cell that communicates with other cells via specialized connections called synapses. Neurons are a major component of the brain. A typical neuron consists of a cell body (soma), dendrites, and a single axon. The axon and dendrites are filaments that extrude from the soma. Dendrites typically branch profusely and extend a few hundred micrometers from the soma. The axon leaves the soma at a swelling called the axon hillock and may branch. At the end of an axon branch is the axon terminal, where the neuron can transmit a signal across the synapse to another cell. In some cases, neurons may lack dendrites or have no axon. The term neurite is used to describe either a dendrite or an axon, particularly when the cell is not a terminally differentiated neuron. A synapse is a structure that permits a neuron to pass an electrical or chemical signal to another neuron or to the target effector cell. Specifically, at a synapse, the plasma membrane of the signal-passing neuron (the presynaptic neuron) comes into close apposition with the membrane of the target (postsynaptic) cell. In many synapses, the presynaptic part is located on an axon and the postsynaptic part is located on a dendrite or soma. However, synapses can connect an axon to another axon or a dendrite to another dendrite. Astrocytes also exchange information with the synaptic neurons, responding to synaptic activity and, in turn, regulating neurotransmission. As used herein, the synapse is preferably a chemical synapse. In a chemical synapse, electrical activity in the presynaptic neuron is converted (via the activation of voltage-gated calcium channels) into the release of a chemical called a neurotransmitter that binds to receptors located in the plasma membrane of the postsynaptic cell. The neurotransmitter may initiate inter alia an electrical response may either excite or inhibit the postsynaptic neuron. Chemical synapses can be classified according to the neurotransmitter released: inter alia glutamatergic (often excitatory) or GABAergic (often inhibitory). Because of the complexity of receptor signal transduction, chemical synapses can have complex effects on the postsynaptic cell. As used herein, the synapse is very preferably a GABAergic synapse. A GABAergic neuron, as used herein, is a neuron which produces GABA and/or comprises GABA-containing synaptic vesicles.

Thus, determining the ratio of the cytosolic GABA level over the mitochondrial GABA level is a highly suitable way to determine the activity of Aralar. Hence, the person skilled in the art has no difficulties in determining whether a certain molecule/drug/compound/composition/pharmaceutical is a modulator of Aralar, i.e. if it is an inhibitor or an activator of said Aralar.

As already indicated above, the surprising identification of Aralar as mitochondrial GABA-transporter in the brain may be exploited to identify molecules, i.e. neuroactive drugs, which modulate the activity of Aralar. As described above, in particular the ratio of the cytosolic GABA level over the mitochondrial GABA level in cells, i.e. in brain cells and/or cells of the neural lineage/neurons is a phenotype which is associated with the activity of Aralar as a mitochondrial GABA-transporter (GABA-transporting activity of Aralar). As also already mentioned above, a variety of well-known assays are available to determine GABA levels separately in mitochondria and the cytosol or synaptic vesicles of cells. Nonetheless, it must be pointed out that the use of Aralar as a potential drug target, i.e. for treating a neurological disorder as described herein, became only apparent upon the surprising identification of Aralar as mitochondrial GABA-transporter in the brain by the inventors. There was no indication in the prior art that modulators of Aralar would modulate the sequestration of GABA into mitochondria, thereby modulating GABA signaling and the behavior.

Thus, in one aspect, the invention relates to a method for identifying a modulator of Aralar, wherein the method comprises
(a) contacting Aralar and/or a cell comprising Aralar with a candidate drug; and
(b) selecting said candidate drug as Aralar modulator when the GABA-transporting activity of said Aralar is altered.

As described herein, i.e. in the context of the screening methods of the invention, the GABA-transporting activity of Aralar may be determined by measuring the GABA level and/or location. In particular, the GABA level refers to the GABA level at a certain subcellular location/compartment, i.e. the mitochondria and/or cytosol/synaptic vesicles (or mimics thereof) as described herein. In the context of the screening methods of the invention, a modulator of Aralar may be considered as a neuroactive drug as provided herein. Similarly, in the context of the screening methods of the invention, a neuroactive drug is preferably a modulator of Aralar.

The inventive screening methods provided herein may be cell- and animal-free and/or comprise a cell, a cell extract and/or a non-human animal as provided herein. Furthermore, the inventive screening methods provided herein may be based on an in vitro assay and/or an in vivo assay. In particular, the inventive screening methods provided herein comprise Aralar and GABA. Thus, the in vitro assay, the cell extract, the cell and/or the non-human animal used in a screening method according to the invention comprises, in particular, GABA and Aralar.

The inventive in vitro assay may be, in particular, cell-free and/or comprise a cell extract as described herein. However, the inventive in vitro assay may further comprise a cell as provided herein in the context of the screening methods of the invention. The inventive in vivo assay comprises a non-human animal (test animal) as provided herein. As further described below, the inventive screening methods comprising a non-human animal as provided herein are neither intended nor suitable for the treatment of the non-human animal by surgery or therapy. Moreover, said inventive screening methods are neither intended nor suitable for diagnosing a disease/disorder in said non-human animal. In the context of the inventive screening methods provided herein, the cell may be comprised in the in vitro assay and/or in the in vivo assay (in the non-human animal).

In one aspect, the invention relates to a method for identifying a neuroactive drug, wherein the method comprises
(a) contacting Aralar and/or a cell comprising Aralar with a candidate drug; and
(b) selecting said candidate drug as Aralar modulator when the GABA-transporting activity of said Aralar is altered.

Furthermore, in one aspect, the invention relates to a method for identifying a modulator of Aralar and/or a neuroactive drug, wherein the method comprises
(a) contacting (i) an in vitro assay containing GABA and Aralar and/or (ii) a cell and/or cell extract containing GABA and Aralar, with a candidate drug; and
(b) selecting said candidate drug as modulator of Aralar and/or a neuroactive drug, when (i) the GABA-transporting activity of Aralar is altered and/or (ii) the level and/or location of GABA within said cell and/or cell extract is altered, respectively.

In particular, said method may further comprise a step between steps (a) and (b) a step of monitoring the level and/or location of GABA within said in vitro assay, monitoring the level and/or location of GABA within said cell and/or cell extract Preferably, said method further comprises in step (a) contacting a non-human animal containing GABA and Aralar with a candidate drug, and in step (b) selecting said candidate drug as modulator of Aralar and/or neuroactive drug when the behavior of said non-human animal is altered, and preferably between steps (a) and (b) a step of monitoring the behavior of said non-human animal.

In one aspect, the invention relates to a method for identifying a modulator of Aralar, wherein the method comprises
(a) contacting (i) an in vitro assay containing GABA and Aralar, (ii) a cell and/or cell extract containing GABA and Aralar and/or (iii) a non-human animal containing GABA and Aralar, with a candidate drug; and
(b) selecting said candidate drug as modulator of Aralar, when (i) the GABA-transporting activity of Aralar is altered, (ii) the level and/or location of GABA within said cell and/or cell extract is altered, and/or (iii) the behavior of said non-human animal is altered, respectively.

In a further aspect, the invention relates to a method for identifying a neuroactive drug, wherein the method comprises
(a) contacting (i) an in vitro assay containing GABA and Aralar, (ii) a cell and/or cell extract containing GABA and Aralar and/or (iii) a non-human animal containing GABA and Aralar, with a candidate drug; and
(b) selecting said candidate drug as neuroactive drug, when (i) the GABA-transporting activity of Aralar is altered, (ii) the level and/or location of GABA within said cell and/or cell extract is altered, and/or (iii) the behavior of said non-human animal is altered, respectively.

In the context of the inventive screening methods provided herein, the level and/or location of GABA is, in particular, regulated by the GABA-transporting activity of Aralar. Thus, the screening methods provided herein may further comprise, for comparison, a step of determining the GABA level/location in a respective in vitro assay/cell extract/cell/non-human animal which has lower or higher levels of GABA and/or Aralar upon contacting with the respective candidate drug. As illustrated in the appended Examples, for example, genetic manipulation of Aralar levels may be employed to verify whether the GABA level and/or location is altered due to the GABA-transporting activity of Aralar.

Similarly, in the context of the inventive screening methods provided herein, the behavior of the non-human animal is, in particular, regulated by the GABA-transporting activity of Aralar. Thus, the screening methods comprising a non-human animal provided herein may further comprise, for comparison, a step of determining the behavior of a respective non-human animal which has lower or higher levels of GABA and/or Aralar, i.e. in the brain, preferably in GABAergic neurons, upon contacting with the respective candidate drug. As illustrated in the appended Examples, for example, genetic manipulation of Aralar levels may be employed to verify whether the behavior of the non-human animal is altered due to the GABA-transporting activity of Aralar.

Preferably, as used herein and in the context of the present invention, i.e. in the context of the inventive screening methods provided herein, i.e. for identifying a neuroactive drug, and/or a modulator, inhibitor or activator of Aralar, the term “identifying” comprises the step of obtaining said neuroactive drug, and/or modulator, inhibitor or activator of Aralar. As regards the medical and non-medical uses of said identified/obtained neuroactive drug, and/or said identified/obtained modulator, inhibitor or activator of Aralar, the same applies as is described herein in the context of the medical and non-medical uses of a modulator of Aralar, respectively. In other words, a modulator, inhibitor or activator of Aralar identified/obtained/obtainable by the inventive screening method(s) provided herein, may be used for treating a neurological disorder as described herein in the context of the modulator, inhibitor or activator of Aralar for use in treating a neurological disorder, respectively. The terms “obtained” and “obtainable” are used interchangeably herein and refer, in the context of the invention, to obtaining the product of a screening method, i.e. an identified modulator of Aralar and/or neuroactive drug.

Thus, in one aspect, the invention relates to a modulator of Aralar for use as a medicament, wherein said modulator is identified/obtained by the inventive screening method(s) provided herein. In one embodiment, said modulator of Aralar is an inhibitor of Aralar.

Thus, in a further aspect, the invention relates to a pharmaceutical composition comprising a modulator of Aralar, wherein said modulator is identified/obtained by the inventive screening method(s) provided herein. In one embodiment, said modulator of Aralar is an inhibitor of Aralar.

Evidently, a plurality of candidate drugs may be employed in the inventive screening methods provided herein to identify/obtain an modulator of Aralar and/or neuroactive drug, i.e. an modulator of Aralar and/or a neuroactive drug that may be used for treating a neurological disorder as provided herein. Furthermore, a particular candidate drug may be tested in the inventive screening methods provided herein to evaluate whether said candidate drug is truly an modulator of Aralar and/or neuroactive drug, or an Aralar inhibitor or an Aralar activator. Therefore, even if said particular candidate drug has been already presumed to be an Aralar modulator, Aralar inhibitor, Aralar activator or neuroactive drug, said candidate drug may be validated with the inventive screening methods provided herein. Thus, the term “screening”, as used herein, i.e. in the context of identifying an modulator of Aralar and/or neuroactive drug, includes the ordinary meanings of the terms “screening”, “identifying”, “evaluating”, “testing”, and “validating”. Thus, the screening methods provided herein may be used for screening drug candidates, identifying an Aralar modulator and/or a neuroactive drug, evaluating/testing whether a candidate drug is an Aralar modulator and/or a neuroactive drug, and validating that a putative Aralar modulator and/or neuroactive drug is truly an Aralar modulator and/or a neuroactive drug.

The term “screening method” or equivalents thereof, as used herein, refers to any of the methods for identifying a modulator of Aralar, or any of the methods for identifying a neuroactive drug according to the invention as provided herein.

The invention further relates to a neuroactive drug for use in treating a neurological disorder according to the invention and/or for use in normalizing the GABA level and/or location in a subject, wherein said neuroactive drug is identified/obtained by a method for identifying a neuroactive drug according to the invention and/or by a method for identifying a modulator of Aralar. As regards the use of said neuroactive drug for treating a neurological disorder or the use of said neuroactive drug in normalizing the GABA level and/or location in a subject, the same applies as is described herein in the context of the modulator of Aralar for use in treating a neurological disorder, or Aralar for use in normalizing the GABA level and/or location in a subject.

The invention also relates to the non-medical use of a neuroactive drug, wherein said neuroactive drug is identified/obtained by the method for identifying/obtaining a neuroactive drug according to the invention. As regards the non-medical use of said neuroactive drug, the same applies as is described herein in the context of the non-medical use of a modulator of Aralar.

Furthermore, the invention also relates to the use of Aralar, an in vitro assay containing GABA and Aralar, a cell containing GABA and Aralar and/or a non-human animal containing GABA and Aralar for identifying an modulator of Aralar and/or a neuroactive drug as described in the context of the screening method according to the invention.

In one embodiment, said cell is a cell extract. Preferably, said cell extract contains mitochondria and/or synaptic vesicles.

A neuroactive drug, as used herein, refers to a molecule, compound, composition, pharmaceutical composition, drug, mixture, pharmaceutical, and/or medicament which may be used for treating a neurological disorder according to the invention and/or for non-medical purposes, i.e. for preventing, alleviating, reversing, and/or stopping at least one behavioral deficit as described herein in the context of the modulator of Aralar. A candidate drug, as used herein, refers to a molecule, compound, composition, pharmaceutical composition, drug, mixture, pharmaceutical, and/or medicament which is tested for its use as an modulator of Aralar and/or a neuroactive drug by the screening methods as described herein.

A cell extract, as used herein, i.e. in the context of the screening methods of the invention, may be a cell lysate, a crude cell extract, or a purified cell extract, wherein a cell lysate may contain most or all components of the lysed cell, and a cell extract may be enriched for components required for an in vitro assay, i.e. inter alia mitochondria, and, for example, inter alia the cytosol and/or synaptic vesicles. Preferably the cell used for preparing the cell extract/lysate is a cell containing Aralar as described herein, preferably a cell of the neural lineage, preferably a neuron, preferably a GABAergic neuron, as described herein. Preferably, said cell extract comprises intact mitochondria. Preferably, said Aralar is located in the membrane of said mitochondria in said cell extract.

In particular, the in vitro assay containing GABA and Aralar as provided herein, i.e. in the context of the screening methods of the invention, may be cell- and animal-free.

The in vitro assay, as provided herein, may be constituted from purified compounds and/or comprise inter alia purified recombinant proteins, i.e. Aralar. In certain embodiments, said in vitro assay may further comprise a cell extract.

In the in vitro assay containing GABA and Aralar, Aralar may be located in mitochondria, a mitochondrial membrane and/or a structure/membrane mimicking a mitochondrial membrane, i.e. a liposome, proteoliposome or membrane bilayer (Scalise (2013), Pharmaceutics 5, 472-497).

The in vitro assay containing GABA and Aralar may be comprised/performed in a vessel, such as inter alia a dish, a well, a flask, a vial or a bottle.

The activity of Aralar, i.e. in the context of the inventive screening methods provided herein, is in particular the GABA-transporting activity.

The GABA-transporting activity of Aralar may be measured, as illustrated in the appended Examples, by determining the level/localization of GABA (or a marker associated with said GABA level/localization) in the in vitro assay. As Aralar is located in mitochondria, a mitochondrial membrane and/or a structure/membrane mimicking a mitochondrial membrane (e.g. proteoliposome/liposome), the assay may be compartmentalized into at least two regions defined by the two sides of the membrane. Thus, the GABA level may be measured in the two regions/compartments upon contacting the assay with a candidate drug. For example, the contents of the two regions may be separated and the GABA levels in said two regions may be analyzed by ordinary means, such as inter alia, ELISA, Western blotting and mass spectrometry, and preferably compared to each other. Furthermore, GABA may be labeled, i.e. with a fluorescent dye or detected with a fluorescent GABA sensor (Marvin (2019) Nat Methods, 16(8):763-770), and the signal/fluorescence may be determined, for example, by fluorescence microscopy or flow cytometry. Furthermore, GABA may be converted to another molecule which may be detected, and/or a byproduct of said reaction may be detected, e.g. NADH. Evidently, the GABA-transporting activity of Aralar contacted with a candidate drug may be compared to a suitable control/reference assay, thereby determining if the GABA-transporting activity of Aralar is altered. In one embodiment, GABA contained in the in vitro assay may be labeled, preferably with a fluorescent dye or detected with a fluorescent GABA sensor (Marvin (2019) Nat Methods, 16(8):763-770). For example, when labeled GABA is added to a cell extract or said cell extracts contains a fluorescent GABA sensor, and said cell extract further contains intact mitochondria or proteoliposomes/liposomes containing Aralar in the membrane, the activity of the GABA transporter Aralar may be determined by quantifying the GABA level within said mitochondria or proteoliposomes/liposomes. The quantification may be performed, inter alia, with flow cytometry or imaging, i.e. when GABA is labeled with a fluorescent dye. A stronger GABA signal in the mitochondria or within proteoliposomes/liposomes may indicate a higher activity of Aralar.

Thus, in one aspect, the invention relates to a method for identifying a modulator of Aralar, wherein the method comprises
(a) contacting an in vitro assay containing GABA and Aralar with a candidate drug, and
(b) selecting said candidate drug as modulator of Aralar, when the GABA-transporting activity of Aralar is altered.

In a further aspect, the invention relates to a method for identifying a neuroactive drug, wherein the method comprises
(a) contacting an in vitro assay containing GABA and Aralar with a candidate drug, and
(b) selecting said candidate drug as neuroactive drug, when the GABA-transporting activity of Aralar is altered.

Thus, in one aspect, the invention relates to a method for identifying a modulator of Aralar and/or a neuroactive drug, wherein the method comprises
(a) contacting an in vitro assay containing GABA and Aralar with a candidate drug,
(b) monitoring the level and/or location of GABA within said in vitro assay, and
(c) selecting said candidate drug as modulator of Aralar and/or neuroactive drug, when the GABA-transporting activity of Aralar is altered.

In one aspect, the invention relates to a method for identifying an inhibitor of Aralar, wherein the method comprises
(a) contacting an in vitro assay containing GABA and Aralar with a candidate drug,
(b) monitoring the level and/or location of GABA within said in vitro assay, and
(c) selecting said candidate drug as Aralar-inhibitor, when the GABA-transporting activity of Aralar is reduced.

In one aspect, the invention relates to a method for identifying an activator of Aralar, wherein the method comprises
(a) contacting an in vitro assay containing GABA and Aralar with a candidate drug,
(b) monitoring the level and/or location of GABA within said in vitro assay, and
(c) selecting said candidate drug as Aralar-activator, when the GABA-transporting activity of Aralar is increased.

In certain embodiments of the inventive screening methods employing an in vitro assay provided herein, the level and/or location of GABA may be indirectly monitored by measuring the level/location of another compound which correlates with the level and/or location of GABA. As described herein and in the appended Examples, NADH may be such a compound which allows to monitor the level/localization of GABA.

In preferred embodiments, as illustrated in the appended Examples, the in vitro assay containing GABA and Aralar further comprises a membrane mimicking a mitochondrial membrane, i.e. a proteoliposome, wherein Aralar is embedded. Preferably said in vitro assay further comprises a GABA transaminase (GABA-T) and a succinic semialdehyde dehydrogenase. Preferably, the GABA transaminase and/or the succinic semialdehyde dehydrogenase are purified soluble proteins. Preferably, said in vitro assay further comprises aspartate, alpha-ketoglutarate and/or NAD+. Preferably, the extracellular part of Aralar is oriented towards the outside of the proteolipsome. Preferably, said GABA is located/enriched outside of the proteoliposome before treating/contacting with a candidate drug. In particular, if said assay comprises NAD+ and a succinic semialdehyde dehydrogenase, Aralar transports GABA into the proteoliposomes, where GABA is converted to succinate with the production of NADH. Said NADH may be measured/monitored by a commercially available kit (e.g. NADH Quantitation Colorimetric Kit, Biovision) using e.g. a microplate reader. In particular, it may be determined whether the candidate drug modulates the GABA-transporting activity of Aralar by comparing the production of NADH/NADH level to a control experiment (i.e. where the candidate drug is replaced by the vehicle/solvent control).

Furthermore, in one aspect the invention relates to a method for identifying a modulator of Aralar, wherein the method comprises
(a) contacting a cell and/or cell extract containing GABA and Aralar and/or a non-human animal containing GABA and Aralar with a candidate drug; and
(b) selecting said candidate drug as modulator of Aralar, when the level and/or location of GABA within said cell and/or cell extract, and/or the behavior of said non-human animal is altered, respectively.

In a further aspect, the invention relates to a method for identifying a neuroactive drug, wherein the method comprises
(a) contacting a GABA-transporter Aralar, a cell and/or cell extract containing GABA and Aralar and/or a non-human animal containing GABA and Aralar with a candidate drug; and
(b) selecting said candidate drug as neuroactive drug, when the activity of said Aralar, the level and/or location of GABA within said cell and/or cell extract, and/or the behavior of said non-human animal is altered, respectively.

As already indicated above, it may be further verified that the neuroactive drug is a modulator of Aralar, by performing additional control experiments with a cell and/or cell extract that has higher or lower Aralar levels. For example, a candidate drug that has been found to alter the level and/or location of GABA within said cell and/or cell extract and is thus selected as neuroactive drug, may be further tested with a cell and/or cell extract lacking Aralar. If said candidate drug/neuroactive drug has no or a lower effect on the level and/or location of GABA within said cell and/or cell extract lacking Aralar, it is further verified that said candidate drug/neuroactive drug is a modulator of Aralar.

Furthermore, it may be further verified that said neuroactive drug is a modulator of Aralar, by performing additional control experiments with a non-human animal that has higher or lower Aralar levels. For example, a candidate drug that has been found to normalize the behavior of the non-human animal and is thus selected as neuroactive drug, may be further tested with a non-human animal lacking Aralar. If said candidate drug/neuroactive drug has no or a lower effect on the behavior of the non-human animal lacking Aralar, it is further verified that said candidate drug/neuroactive drug is a modulator of Aralar.

Furthermore, in one aspect, the invention relates to a method for identifying a modulator of Aralar, wherein the method comprises
(a) contacting a cell containing GABA and Aralar with a candidate drug; and
(b) selecting said candidate drug as modulator of Aralar, when the level and/or location of GABA within said cell is altered.

In one embodiment, said cell is a cell extract. Preferably, said cell extract contains mitochondria and/or synaptic vesicles.

Thus, in one aspect, the invention further relates to a method for identifying a neuroactive drug, wherein the method comprises
(a) contacting a cell containing GABA and Aralar with a candidate drug; and
(b) selecting said candidate drug as neuroactive drug, when the level and/or location of GABA within said cell is altered.

In one embodiment, said cell is a cell extract. Preferably, said cell extract contains mitochondria and/or synaptic vesicles.

As regards the cell containing GABA and Aralar in the context of the screening methods according to the invention, the same applies as is described above in the context of the cell that is used for determining whether a molecule is a modulator of Aralar. In brief, the cell containing GABA and Aralar, as used herein, may be any cell containing GABA and Aralar as described above. Preferably, said cell is a cell of the neural lineage, preferably a neuron, preferably a GABAergic neuron, as described herein. Said cell containing Aralar and GABA may further have an insertion, deletion, premature stop codon, and/or frame-shift mutation within the Cyfip1 gene and or is derived from a subject with Cyfip1 haploinsufficiency and/or a subject with impaired behavior. As regards the behavior, the same applies as is described below in the context of normalizing the behavior of the non-human animal. Preferably, said cell containing GABA and Aralar is maintained in vitro or ex vivo in culture as described above. Preferably, said cell containing GABA and Aralar is a human cell, a mouse cell or an insect cell, preferably a human cell.

However, in certain embodiments, said cell is not maintained in vitro or ex vivo in culture before and during contacting with the candidate drug, but comprised in a non-human animal (test animal) before and during contacting with the candidate drug. In that case, the cell, or preferably the tissue such as inter alia the brain or a specific region of the brain comprising said cell, may be extracted/explanted from said non-human animal. Then, the monitoring/measuring/determining the GABA level in the mitochondria, and/or cytosol and/or synaptic vesicles or determining the ratio of the cytosolic GABA level over the mitochondrial GABA level may be performed, as described herein, on said extracted/explanted cell/tissue. For immunochemistry measurements, said tissue explant may be cut into thin sections before monitoring/measuring/determining the GABA level and/or location in the cell comprised therein. As regards the non-human animal, contacting said non-human animal with the candidate drug, the tissue and the extraction/explantation of said tissue from the non-human animal, the same applies as is described in more detail further below in the context of the screening method, wherein said method comprises a step of contacting a non-human animal containing GABA and Aralar with a candidate drug according to the invention.

The level of GABA in the cell and/or cell extract containing GABA and Aralar according to the invention may be the GABA level in the mitochondria, and/or the GABA level in the cytosol and/or synaptic vesicles. Furthermore, the location of GABA in said cell may be the mitochondria and/or the cytosol or synaptic vesicles of said cell. Typically, when the location of GABA changes, i.e. when GABA is transported from the cytosol/synaptic vesicles into the mitochondria, also the level of GABA changes at said locations.

Thus, evaluating the alteration of the level and/or location of GABA comprises, in particular, determining the GABA level in the mitochondria and/or cytosol or synaptic vesicles of said cell, preferably determining the ratio of the cytosolic GABA level over the mitochondrial GABA level.

The term “monitoring”, as used herein, refers to taking at least one measurement at at least one time-point after contacting a cell and/or non-human animal with a candidate drug.

Hence, preferably said method for identifying a neuroactive drug and/or said method for identifying a modulator of Aralar comprising a step of contacting a cell containing GABA and Aralar with a candidate drug further comprises a step of monitoring the level and/or location of GABA within said cell after the contacting. The monitoring of the level and/or location of GABA within said cell allows to determine the GABA level in the mitochondria, and/or cytosol and/or synaptic vesicles and/or determine the ratio of the cytosolic GABA level over the mitochondrial GABA level. For contacting the cell with the candidate drug, the cell must be alive. However, for monitoring/measuring/determining the level and/or location of GABA within said cell, said cell may be alive and/or said cell may have been fixed and/or lysed. When the cell is alive, the GABA level and/or location may be measured at several time-points before and/or after contacting said cell with the candidate drug.

As regards monitoring/measuring/determining the GABA level in the mitochondria, and/or cytosol and/or synaptic vesicles or determining the ratio of the cytosolic GABA level over the mitochondrial GABA level, the same applies as is described above in the context of measuring GABA levels at subcellular resolution, i.e. by immunochemistry and/or by fractionating the cell material to enrich for mitochondria or cytosol/synaptic vesicles and subsequently performing a GABA ELISA Enzyme immunoassay. In other words, the same means and methods described herein in the context for determining whether a molecule is a modulator of Aralar can be also used in the screening methods according to the invention.

Thus, in one aspect, the invention relates to a method for identifying a modulator of Aralar, wherein the method comprises
(a) contacting a cell containing GABA and Aralar with a candidate drug;
(b) monitoring the level and/or location of GABA within said cell, and
(c) selecting said candidate drug as modulator of Aralar, when the level and/or location of GABA within said cell is altered.

In one embodiment, said cell is a cell extract. Preferably, said cell extract contains mitochondria and/or synaptic vesicles.

Thus, in one aspect, the invention relates to a method for identifying a neuroactive drug, wherein the method comprises
(a) contacting a cell containing GABA and Aralar with a candidate drug;
(b) monitoring the level and/or location of GABA within said cell; and
(b) selecting said candidate drug as neuroactive drug, when the level and/or location of GABA within said cell is altered.

In one embodiment, said cell is a cell extract. Preferably, said cell extract contains mitochondria and/or synaptic vesicles.

Furthermore, the alteration of the level and/or location of GABA as described herein, i.e. in the context of the screening methods of the invention, can be readily evaluated by comparing the respective level and/or location of GABA before and after contacting the cell containing GABA and Aralar with the candidate drug, and/or, preferably, by comparing the respective level and/or location of GABA in the cell that is contacted with the candidate drug to a similar control cell that is not contacted with the candidate drug but otherwise treated the same way. In particular, when the GABA level and/or location is compared to a control cell, and thus measuring the GABA level and/or location before the contacting with the candidate drug is not required, inter alia the immunochemistry methods and/or cell fractioning methods in combination with an immunoassay/GABA ELISA as described above may be employed. When the level and/or location of GABA is measured before and after contacting said cell with the candidate drug, said cell must be alive at least for the measurements taken before the contacting with the candidate drug. In that case, the GABA levels and/or localization must be visualized, at least before contacting with the candidate drug, by live-imaging, for example inter alia by magnetic resonance spectrometry as described above, by using a fluorescent antibody/nanobody/chromobody which is directed against GABA and which is coupled to a fluorescent dye or fluorescent protein, and/or by using a fluorescent GABA sensor (Marvin (2019) Nat Methods, 16(8):763-770). Said antibody/nanobody/chromobody may be directly introduced in the cell, i.e. by transfection, lipofection and/or electroporation, or may be encoded by a nucleic acid molecule which is introduced into the cell by similar means.

When the candidate drug is selected as a neuroactive drug according to the invention because it has been found to (i) increase GABA levels in the cytosol and/or synaptic vesicles, (ii) decrease GABA levels in the mitochondria, and/or (iii) increase the ratio of the cytosolic GABA level over the mitochondrial GABA level, said drug is considered as an inhibitor of Aralar and hence may be used for treating a neurological disorder and/or for non-medical purposes as described above in the context of an inhibitor of Aralar.

Thus, in one aspect, the invention relates to a method for identifying an inhibitor of Aralar, wherein the method comprises
(a) contacting a cell containing GABA and Aralar with a candidate drug;
(b) monitoring the level and/or location of GABA within said cell, and
(c) selecting said candidate drug as Aralar-inhibitor, when
   (i) GABA levels in the cytosol and/or synaptic vesicles are increased,
   (ii) GABA levels in the mitochondria are decreased, and/or
   (iii) the ratio of the cytosolic GABA level over the mitochondrial GABA level is increased.

In one embodiment, said cell is a cell extract. Preferably, said cell extract contains mitochondria and/or synaptic vesicles.

Thus, in one aspect, the invention relates to a method for identifying a neuroactive drug, wherein the method comprises
(a) contacting a cell containing GABA and Aralar with a candidate drug;
(b) monitoring the level and/or location of GABA within said cell; and
(b) selecting said candidate drug as neuroactive drug, when
   (i) GABA levels in the cytosol and/or synaptic vesicles are increased,
   (ii) GABA levels in the mitochondria are decreased, and/or
   (iii) the ratio of the cytosolic GABA level over the mitochondrial GABA level is increased.

Said neuroactive drug is, in particular, also an Aralar-inhibitor, and hence may be used for treating a neurological disorder and/or for non-medical purposes as described above in the context of an inhibitor of Aralar.

In one embodiment, said cell is a cell extract. Preferably, said cell extract contains mitochondria and/or synaptic vesicles.

When the candidate drug is selected as a neuroactive drug according to the invention because it has been found to (i) decrease GABA levels in the cytosol and/or synaptic vesicles, (ii) increase GABA levels in the mitochondria, and/or (iii) decrease the ratio of the cytosolic GABA level over the mitochondrial GABA level, said drug is considered as an activator of Aralar and hence may be used for treating a neurological disorder and/or for non-medical purposes as described above in the context of an activator of Aralar.

Thus, in one aspect, the invention relates to a method for identifying an activator of Aralar, wherein the method comprises
(a) contacting a cell containing GABA and Aralar with a candidate drug;
(b) monitoring the level and/or location of GABA within said cell, and
(c) selecting said candidate drug as Aralar-activator, when
   (i) GABA levels in the cytosol and/or synaptic vesicles are decreased,
   (ii) GABA levels in the mitochondria are increased, and/or
   (iii) the ratio of the cytosolic GABA level over the mitochondrial GABA level is decreased.

In one embodiment, said cell is a cell extract. Preferably, said cell extract contains mitochondria and/or synaptic vesicles.

Thus, in one aspect, the invention relates to a method for identifying a neuroactive drug, wherein the method comprises
(a) contacting a cell containing GABA and Aralar with a candidate drug;
(b) monitoring the level and/or location of GABA within said cell; and
(b) selecting said candidate drug as neuroactive drug, when
   (i) GABA levels in the cytosol and/or synaptic vesicles are decreased,
   (ii) GABA levels in the mitochondria are increased, and/or
   (iii) the ratio of the cytosolic GABA level over the mitochondrial GABA level is decreased.

Said neuroactive drug is, in particular, also an Aralar-activator, and hence may be used for treating a neurological disorder and/or for non-medical purposes as described above in the context of an activator of Aralar.

In one embodiment, said cell is a cell extract. Preferably, said cell extract contains mitochondria and/or synaptic vesicles.

As illustrated in the appended Examples, a non-human animal may be used for identifying a modulator of Aralar and/or a neuroactive drug.

Thus, in a further aspect, the invention relates to a method for identifying a modulator of Aralar, wherein the method comprises
(a) contacting a non-human animal containing GABA and Aralar with a candidate drug; and
(b) selecting said candidate drug as modulator of Aralar, when the level and/or location of GABA within a cell comprised in said non-human animal is altered, and/or when the behavior of said non-human animal is altered.

In one embodiment, said cell is a cell extract obtained from said cell. Preferably, said cell extract contains mitochondria and/or synaptic vesicles.

In a further aspect, the invention relates to a method for identifying a neuroactive drug, wherein the method comprises
(a) contacting a non-human animal containing GABA and Aralar with a candidate drug; and
(b) selecting said candidate drug as neuroactive drug, when the level and/or location of GABA within a cell comprised in said non-human animal is altered, and/or when the behavior of said non-human animal is altered.

As regards the cell comprised in the non-human animal, evaluating the alteration of the level and/or location of GABA, and the monitoring/measuring/determining the GABA level in the mitochondria, and/or cytosol and/or synaptic vesicles or determining the ratio of the cytosolic GABA level over the mitochondrial GABA level, the same applies as is described above in the context of the method for identifying a neuroactive drug or the method for identifying a modulator of Aralar, wherein said method comprises a step of contacting a cell containing GABA and Aralar with a candidate drug according to the invention (and the parts of the specification referenced in said context), and in particular to the embodiments wherein said cell is not maintained in vitro or ex vivo in culture before and during contacting with the candidate drug, but comprised in a non-human animal (test animal) before and during contacting with the candidate drug.

A non-human animal, as used herein, refers to a test animal which may be used inter alia for determining the activity of Aralar, determining whether a molecule is a modulator, i.e. an inhibitor or activator, of Aralar, evaluating the alteration of the level and/or location of GABA, monitoring/measuring/determining the GABA level in the mitochondria, and/or cytosol and/or synaptic vesicles or determining the ratio of the cytosolic GABA level over the mitochondrial GABA level, evaluating the alteration of the behavior of a non-human animal, and/or identifying a neuroactive drug according to the invention and as described herein. Said non-human animal is not particularly limited to a specific animal but should have a central nervous system and/or a social behavior, preferably both. Thus, said non-human animal may be, for example, inter alia an insect, a mollusk, or a vertebrate. Preferably, said non-human animal is a fly, a fish, a frog, a mouse, a rat, or a monkey. Very preferably, said non-human animal is a fly or a mouse, and most preferably a fly. Preferably, said fly is Drosophila, preferably Drosophila melanogaster.

Furthermore, said non-human animal contains GABA and Aralar, in particular in the central nervous system (CNS), preferably the brain. In particular, GABA and Aralar are contained in a cell of the CNS and/or the brain, preferably in a neuron, preferably a GABAergic neuron. This means that GABA and Aralar molecules are present in said cell. Because Aralar must be present at the protein level, this may be verified by determining Aralar expression at the protein level in that cell. However, since Aralar expression at the protein level requires expression of Aralar at the mRNA level, the presence of Aralar may be also determined at the mRNA level/level of gene expression.

Preferably, said non-human animal (test animal) further has a Cytoplasmic FMR1 Interacting Protein 1 (Cyfip1) haploinsufficiency, preferably due to an insertion, deletion, premature stop codon, and/or frame-shift mutation within the Cyfip1 gene. A fly, i.e. Drosophila, with Cyfip1 haploinsufficiency may be, in particular, a Cyfip^{85.1}/+ fly as illustrated in the appended Examples. As further illustrated in the appended Examples, the non-human animal, i.e. a fly, may also be a Fmr1 mutant, e.g. have no functional Fmr1 allele. A test animal having a Cyfip1 haploinsufficiency (or a Fmr1 mutation) is particularly suitable for determining if a candidate drug normalizes or ameliorates the behavior because, as also illustrated in the Examples, Cyfip1 haploinsufficiency (or a Fmr1 mutation) is associated and/or leads to deficits in behavior in a variety of animals including flies, mice and humans.

Thus, in preferred embodiments, the non-human animal has an impaired/abnormal behavior, preferably wherein said non-human animal comprises a modified Cyfip1 or a modified Fmr1, i.e. a modified Cyfip1, more preferably wherein the non-human animal comprises an insertion, deletion, premature stop codon, and/or frame-shift mutation within said Cyfip1 gene. In certain embodiments, the mutation, i.e. an insertion/deletion, may be comprised in the regulatory elements of the Cyfip1 gene.

Preferably, the non-human animal (test animal) has altered GABA levels, in particular in the brain, preferably in neurons, preferably GABAergic neurons, as is further illustrated in the appended Examples. The GABA levels may be altered/different compared to a control and/or may be determined in the mitochondria, the cytosol, synaptic vesicles and/or synapses, in particular in the brain, and/or in the cerebrospinal fluid, in the blood and/or in the urine of said non-human animal.

As used herein and in the context of the invention, the non-human animal (test animal) is not treated by surgery or therapy and is not diagnosed for having a disease/disorder. In the context of the inventive screening methods provided herein, the non-human animal is contacted with a candidate drug for testing/evaluating said candidate drug and not as a method of treatment (i.e. not as a medicament). It may be verified for experimental purposes that the non-human animal (test animal), e.g. a transgenic test animal, has a certain disorder/condition/disorder, for example that Cyfip haploinsufficient flies have behavioral deficits, but the inventive screening methods provided herein do not comprise diagnosing the non-human animal (test animal) in a medical/therapeutic sense, i.e. the screening methods are not suitable for indicating a certain method of treatment or providing a prognosis of the disorder/condition/disorder.

The use of a non-human animal (test animal) in a screening method according to the invention is ethically acceptable because it is evident that the development of therapies for treating a neurological disorder, i.e. a neurodevelopmental and/or psychiatric disorder, requires animal experimentation before clinical trials with humans can be started. It is also evident that the inventive screening method provided herein are not particularly harmful for the test animal and can readily applied/adjusted in agreement with animal rights laws. Evidently, animal suffering should be minimized when performing the screening methods.

Thus, the non-human animal which is used as a test animal and/or in a screening method according to the invention, must not be confused with treating a subject according to the invention and as described herein. The subject to be treated according to the invention is, as already described above, i.e. a human, a domestic animal, a pet or, most preferably, a human patient.

The non-human animal according to the invention may be contacted with the candidate drug inter alia by mixing said candidate drug with the food that is fed to said non-human animal, and/or by injecting or infusing said candidate drug into said non-human animal, for example, inter alia into the blood, a tissue such as inter alia the skin, or a muscle, the cerebrospinal fluid, and/or subcutaneously.

Preferably, in the context of said method for identifying a neuroactive drug, wherein said method comprises contacting a non-human animal containing GABA and Aralar with a candidate drug, said candidate drug is selected as neuroactive drug, when the behavior of said non-human animal is altered. Preferably, said candidate drug is selected when the behavior of said non-human animal is ameliorated or normalized, in particular when it is normalized.

In one aspect, the invention relates to a method for identifying a modulator of Aralar, wherein the method comprises
(a) contacting a non-human animal containing GABA and Aralar with a candidate drug; and
(b) selecting said candidate drug as modulator of Aralar, when the behavior of said non-human animal is altered.

In one aspect, the invention relates to a method for identifying a neuroactive drug, wherein the method comprises
(a) contacting a non-human animal containing GABA and Aralar with a candidate drug; and
(b) selecting said candidate drug as neuroactive drug, when the behavior of said non-human animal is altered.

Thus, preferably, said method for identifying a neuroactive drug comprising a step of contacting a non-human animal containing GABA and Aralar with a candidate drug, further comprises a step of monitoring the behavior of said non-human animal after the contacting. The behavior of said non-human animal can be readily evaluated by comparing the respective behavior before and after contacting the non-human animal with the candidate drug, and/or, preferably, by comparing the respective behavior of the non-human animal that is contacted with the candidate drug to a similar control non-human animal that is not contacted with the candidate drug but otherwise treated the same way.

Thus, in one aspect, the invention relates to a method for identifying a modulator of Aralar, wherein the method comprises
(a) contacting a non-human animal containing GABA and Aralar with a candidate drug;
(b) monitoring the behavior of said non-human animal; and
(c) selecting said candidate drug as modulator of Aralar, when the behavior of said non-human animal is altered.

Thus, in one aspect, the invention relates to a method for identifying a neuroactive drug, wherein the method comprises
(a) contacting a non-human animal containing GABA and Aralar with a candidate drug;
(b) monitoring the behavior of said non-human animal; and
(c) selecting said candidate drug as neuroactive drug, when the behavior of said non-human animal is altered.

As indicated above, it may be verified, in the context of the inventive screening methods provided herein, by further control experiments that the candidate drug/neuroactive drug alters the behavior of the non-human animal by modulating the GABA-transporting activity of Aralar.

The behavior of the non-human animal/test animal according to the invention reflects in particular a behavior that is altered in human patients which have ASD and/or schizophrenia, and/or which have behavioral deficits as described above. Evidently, the behavior of the non-human animal (test animal) is not identical to a human subject but the test animal according to the invention and humans usually have certain aspects of behavior in common. Furthermore, certain genes and molecular mechanisms underlying a certain behavior may be, at least partly, similar between said test animal and humans. Typical common aspects of behavior that may be measured with a test animal, are inter alia, social interactions, anxiety, motivation, extroversion/introversion, courtship behavior, social space behavior, grooming, social memory, and/or social dominance.

At least for typical model organisms/test animals, in particular for flies and mice, suitable assays to measure said common aspects of behavior, are readily available in the art. As illustrated in the appended Examples, to determine the behavior according to the invention in flies, for example, the social events between flies may be counted, i.e. in a food-competition assay, the courtship index may be determined, i.e. in a pair-mating assay, the social space behavior may be assessed, and/or or the grooming behavior may be determined.

To determine the social behavior according to the invention in mice, for example, inter alia the following tests may be employed: Test for Sociability in the Three-Chamber apparatus, Social Novelty Test, Free play Test, Ultrasonic vocalization Test, Social/conditioned place preference Test, and/or Resident intruder Test (Crawley (2007), Brain Pathology 17(4), 448-459; Lai (2014), Current Pharmaceutical Design, 20(32), 5139-5150; Portfors (2007), JAALAS, 46(1), 28-34; Ricceri (2007), Behavioural Brain Research, 176(1), 40-52; Silverman (2010), Nature Reviews. Neuroscience, 11(7), 490-502; Toth (2013), Cell and Tissue Research, 354(1), 107-118; and Wang (2014), Trends in Neurosciences, 37(11), 674-682).

As already mentioned above, the term “normalizing”, as used herein, refers to the establishment or re-establishment of the normal condition, wherein the normal condition is the respective condition of the control group as described herein. The term “ameliorating”, as used herein, refers to the establishment or re-establishment of a condition which is more similar to the normal condition, i.e. as compared to before an intervention, wherein the normal condition is the respective condition of the control group as described herein. Thus, the same applies for ameliorating the behavior as for normalizing the behavior, except that for an amelioration, the behavior is more similar to, but not necessarily undistinguishable from the normal behavior.

In particular, normalizing the behavior of the non-human animal refers to the (re-) establishment of a normal behavior (reference behavior) for at least one relevant aspect of behavior which i.e. is related to a deficit in behavior according to the invention, preferably in a subject having an altered behavior as described herein. Preferably, a relevant aspect of behavior is, in particular if said non-human animal is a fly, and as illustrated in the Examples, the social interaction with other animals of the same species/strain, the courtship behavior, and/or the grooming behavior. If said non-human animal is a mouse, a relevant aspect of behavior may be the sociability. As mentioned above, it is well known in the art how to determine a suitable control group. In the case of test animals, it may be preferable to use a normal, healthy, inbred wild-type animal strain, in particular if the test animal is a mutant of said strain. Moreover, the subjects of said control group and/or a reference subject do inter alia not have a neurological disorder, in particular not a neurodevelopmental disorder, as described herein. Furthermore, said subjects of said control group and/or said reference subject do not have deficits which resemble the behavioral deficits as described herein. In other words, the subjects of said control group and/or a reference subject have a normal behavior.

For example, the behavior of flies having reduced social interactions, a reduced courtship behavior, and/or increased grooming behavior is considered to be normalized, when they show, after contacting with a candidate drug, an amount of social interactions, a courtship behavior, and or a grooming behavior which is similar to flies of a wild-type fly strain which are considered to have a normal behavior. Furthermore, the behavior of flies having reduced social interactions, a reduced courtship behavior, and/or increased grooming behavior is considered to be ameliorated, when they show, after contacting with a candidate drug, an amount of social interactions, a courtship behavior, and/or a grooming behavior which is more similar to said flies of a wild-type fly strain, than bevor contacting with said candidate drug.

Thus, in one aspect, the invention relates to a method for identifying an inhibitor of Aralar, wherein the method comprises
(a) contacting a non-human animal containing GABA and Aralar with a candidate drug;
(b) monitoring the behavior of said non-human animal; and
(c) selecting said candidate drug as Aralar-inhibitor, when the behavior of said non-human animal is normalized, wherein said non-human animal has
   (i) reduced GABA levels in the cytosol, synaptic vesicles, synapses, the cerebrospinal fluid, blood and/or urine,
   (ii) increased GABA levels in the mitochondria, and/or
   (iii) a mutation in the FMR1 gene or a Cyfip1 haploinsufficiency, preferably a Cyfip1 haploinsufficiency.

Thus, in one aspect, the invention relates to a method for identifying a neuroactive drug, wherein the method comprises
(a) contacting a non-human animal containing GABA and Aralar with a candidate drug;
(b) monitoring the behavior of said non-human animal; and
(c) selecting said candidate drug as neuroactive drug, when the behavior of said non-human animal is normalized, wherein said non-human animal has
   (i) reduced GABA levels in the cytosol, synaptic vesicles, synapses, the cerebrospinal fluid, blood and/or urine,
   (ii) increased GABA levels in the mitochondria, and/or
   (iii) a mutation in the FMR1 gene or a Cyfip1 haploinsufficiency, preferably a Cyfip1 haploinsufficiency.

Said neuroactive drug is, in particular, also an Aralar-inhibitor, and hence may be used for treating a neurological disorder and/or for non-medical purposes as described above in the context of an inhibitor of Aralar.

In a further aspect, the invention relates to a method for identifying a modulator of Aralar and/or a neuroactive drug, wherein the method comprises
(a) contacting a non-human animal containing GABA and Aralar with a candidate drug;
(b) monitoring the level and/or location of GABA within a cell comprised in said non-human animal, and monitoring the behavior of said non-human animal; and
(c) selecting said candidate drug as modulator of Aralar and/or neuroactive drug, when the level and/or location of GABA within said cell is altered, and when the behavior of said non-human animal is normalized.

In particular, in step (a), the central nervous system, i.e. the brain, of said non-human animal is contacted with said candidate drug, and said cell is a cell of said central nervous system/brain, preferably a neuron, preferably a GABAergic neuron.

In one embodiment, said cell is a cell extract obtained from said cell. Preferably, said cell extract contains mitochondria and/or synaptic vesicles.

In one embodiment, said cell is a cell extract. Preferably, said cell extract contains mitochondria and/or synaptic vesicles.

In a further aspect, the invention relates to a method for identifying an inhibitor of Aralar and/or a neuroactive drug, wherein the method comprises
(a) contacting a non-human animal containing GABA and Aralar with a candidate drug;
(b) monitoring the level and/or location of GABA within a cell comprised in said non-human animal, and monitoring the behavior of said non-human animal; and
(c) selecting said candidate drug as Aralar-inhibitor/neuroactive drug, when
   (i) GABA levels in the cytosol and/or synaptic vesicles are increased,
   (ii) GABA levels in the mitochondria are decreased, and/or
   (iii) the ratio of the cytosolic GABA level over the mitochondrial GABA level is increased, and when the behavior of said non-human animal is normalized, wherein said non-human animal has
   (iv) reduced GABA levels in the cytosol, synaptic vesicles, synapses, the cerebrospinal fluid, blood and/or urine,
   (v) increased GABA levels in the mitochondria, and/or
   (vi) a mutation in the FMR1 gene or a Cyfip1 haploinsufficiency, preferably a Cyfip1 haploinsufficiency.

In particular, in step (a), the central nervous system, i.e. the brain, of said non-human animal is contacted with said candidate drug, and said cell is a cell of said central nervous system/brain, preferably a neuron, preferably a GABAergic neuron.

In one embodiment, said cell is a cell extract obtained from said cell. Preferably, said cell extract contains mitochondria and/or synaptic vesicles.

In particular, when the candidate drug is selected as a neuroactive drug and/or as Aralar-inhibitor according to the invention because said drug has been found to
(i) increase the GABA levels in the cytosol, synaptic vesicles, synapses, the cerebrospinal fluid, blood, plasma and/or urine,
(ii) decrease the GABA levels in the mitochondria,
(iii) normalize or ameliorate deficits in behavior/impaired behavior, in particular, a behavior which resembles the behavior of human patients which have ASD and/or schizophrenia, and/or, a behavior characterized by at least 1, 2, 4, 8 or 12 (in this order of preference) deficit(s) in behavior as described herein in the context of the non-medical use of a modulator of Aralar, and/or,
(iv) normalize the social interactions with a non-human animal of the same species/strain, the courtship behavior and/or the grooming behavior
   in a non-human animal which has
(i) reduced GABA levels in the cytosol, synaptic vesicles, synapses, the cerebrospinal fluid, blood and/or urine,
(ii) increased GABA levels in the mitochondria, and/or
(iii) a mutation in the FMR1 gene or a Cyfip1 haploinsufficiency, preferably a Cyfip1 haploinsufficiency,
   said drug is considered as an inhibitor of Aralar and hence may be used for treating a neurological disorder and/or for non-medical purposes as described above in the context of an inhibitor of Aralar.

In particular, when the candidate drug is selected as a neuroactive drug and/or as Aralar-activator according to the invention because said drug has been found to
(i) decrease the GABA levels in the cytosol, synaptic vesicles, synapses, the cerebrospinal fluid, blood, plasma and/or urine,
(ii) increase the GABA levels in the mitochondria,
(iii) normalize or ameliorate deficits in behavior/impaired behavior, in particular, a behavior which resembles the behavior of human patients which have ASD and/or schizophrenia, and/or, a behavior characterized by at least 1, 2, 4, 8 or 12 (in this order of preference) deficit(s) in behavior as described herein in the context of the non-medical use of a modulator of Aralar, and/or,
(iv) normalize the social interactions with a non-human animal of the same species/strain, the courtship behavior and/or the grooming behavior
   in a non-human animal which has
(i) increased GABA levels in the cytosol, synaptic vesicles, synapses, the cerebrospinal fluid, blood and/or urine,
(ii) decreased GABA levels in the mitochondria, and/or
(iii) an IDH haploinsufficiency, and/or an Aralar insufficiency, said drug is considered as an activator of Aralar and hence may be used for treating a neurological disorder and/or for non-medical purposes as described above in the context of an activator of Aralar.

In further aspects, the invention relates to a method for identifying a neuroactive drug, wherein the method comprises
(a) contacting a GABA-transporter Aralar with a candidate drug; and
(b) selecting said candidate drug when the activity of said Aralar is altered.

Said method is preferably an in vitro method wherein Aralar is present outside of a cell or a non-human animal. In particular, said Aralar is the Aralar protein. Said GABA-transporter Aralar may be purified or comprised in a cell extract. The activity of Aralar in the context of said cell- and animal-free in vitro screening method, is the enzymatic activity of Aralar, in particular its GABA-transporting activity. Preferably, said in vitro method is performed in a vessel, such as inter alia a dish, a well, a flask or a bottle.

In certain embodiments, the said cell- and animal-free in vitro screening method according to the invention comprises purified Aralar and GABA, preferably labeled GABA. Preferably, said purified Aralar is located in a mitochondrial membrane or a structure/membrane mimicking a mitochondrial membrane. Preferably, the activity of said Aralar is determined by measuring the translocation of GABA across said membrane.

In particular, said cell extract is a lysate or extract of a cell containing Aralar as described herein, preferably a cell of the neural lineage, preferably a neuron, preferably a GABAergic neuron, as described herein. Preferably, said cell extract comprises intact mitochondria. Preferably, said Aralar is located in the membrane of said mitochondria.

In particular, if said method comprises a cell extract as provided herein, the enzymatic activity of Aralar may be measured, for example, by adding labeled GABA to said cell extract containing intact mitochondria which contain Aralar, i.e. in the membrane. GABA may be labeled, for example, by conjugating with a fluorescent dye. Furthermore, said cell extract may comprise a fluorescent GABA sensor (Marvin (2019) Nat Methods, 16(8):763-770) to quantify GABA levels/localization. The activity of the GABA transporter Aralar may be determined by quantifying the GABA level within said mitochondria. The quantification may be performed, inter alia, with flow cytometry or imaging, i.e. when GABA is labeled with a fluorescent dye or detected with a fluorescent GABA sensor. A stronger GABA signal in the mitochondria may indicate a higher activity of Aralar.

Preferably, said method for identifying a neuroactive drug comprising a step of contacting a GABA-transporter Aralar with a candidate drug further comprises a step of monitoring the enzymatic activity of said Aralar after the contacting. Since, said method is a cell and animal-free in vitro method, measurements may be done before and after the contacting with the candidate peptide by taking samples from said vessel.

In particular, when the candidate drug is selected as a neuroactive drug according to the invention because said neuroactive drug has been found to decrease the enzymatic activity of Aralar, said drug is considered as an inhibitor of Aralar and hence may be used for treating a neurological disorder and/or for non-medical purposes as described above in the context of an inhibitor of Aralar.

In particular, when the candidate drug is selected as a neuroactive drug according to the invention because said neuroactive drug has been found to increase the enzymatic activity of Aralar, said drug is considered as an activator of Aralar and hence may be used for treating a neurological disorder and/or for non-medical purposes as described above in the context of an activator of Aralar.

In one aspect, the invention relates to a method for identifying a modulator of Aralar and/or a neuroactive drug, wherein the method comprises
(a) contacting a cell extract containing a GABA-transporter Aralar with a candidate drug; and
(b) selecting said candidate drug as Arlar-modulator/neuroactive drug when the GABA-transporting activity of said Aralar is altered.

In one aspect, the invention relates to a method for identifying a modulator of Aralar and/or a neuroactive drug, wherein the method comprises
(a) contacting a cell extract containing mitochondria and a GABA-transporter Aralar with a candidate drug;
(b) quantifying the GABA level within said mitochondria, and
(c) selecting said candidate drug as modulator of Aralar and/or neuroactive drug when the GABA level in the mitochondria is altered.

The invention is also characterized by the following figures, figure legends and the following non-limiting examples.

### BRIEF DESCRIPTION OF THE FIGURES

FIG. 1. Deficits in behavior in flies with Cyfip haploinsufficiency. (A) Control (w1118), Cyfip^{85.1}/+, TM3/+ and Cyfip^{85.1}/TM3 flies were analyzed for total number of social interactions in a competition for food assay (n>20 groups of 8 flies) or (B) in a courtship assay (n>20). For both paradigms data are shown with box and whiskers graphs where the line inside the box indicates the median, ****p<0.0001, ***p<0.001, **p<0.01, Kruskal-Wallis test, Dunn's multiple comparison test. TM3 refers to a different genetic background than w1118. This confirms that the behavioral deficits were not due to the genetic background but due to the Cyfip haploinsufficiency. (C) Adult-specific abrogation of Cyfip (ElavGal4;TubGal80^{ts}>UAS-CYFIP-IR 29° C.) pan-neuronally compared with uninduced (ElavGal4;TubGal80^{ts}>UAS-CYFIP-IR 18° C.), transgene-alone (UAS-CYFIP-IR/+) and driver-alone (ElavGal4;TubGal80^{ts}/+) control flies were analyzed for total number of social interactions. Conditional expression of Cyfip in the mutant background (genetic rescue, ElavGal4;TubGal80^{ts}>UAS-CYFIP,Cyfip^{85.1} 29° C.) in comparison with the uninduced transgene (ElavGal4;TubGal80^{ts}>UAS-CYFIP,Cyfip^{85.1} 18° C.) reverses social defects of CYFIP^{85.1}/+ flies. n=14 independent experiments with 8 flies per genotype, ****p<0.0001, **p<0.01, Kruskal-Wallis test, Dunn's multiple comparison test. (D) Cyfip mRNA levels in adult brains upon pan-neuronal Cyfip knockdown (29° C.) in comparison to parental control strains. The levels of Cyfip were normalized to rpl13 mRNA. n=3 for each genotype, mean±s.e.m. *p<0.05, Kruskal-Wallis test, Dunn's multiple comparison test. (E) Female Cyfip85.1/+ flies show a decreased competition for food. Control (w1118) and Cyfip85.1/+ female flies were analyzed for total number of social interactions in a competition for food assay. n>12 groups of 8 flies. Data are shown with box and whisker plots where the line inside the box indicates the median, ***p<0.001, Mann-Whitney test. (H) Male Cyfip85.1/+ flies are hyperactive. Locomotion activity of control and Cyfip85.1/+ flies was monitored over 24 hours (left) and during the morning period ZT0-ZT6 (right). n>40 flies for each genotype were tested. mean±s.e.m., **p<0.01, ns, not significant, Mann-Whitney test. (I) No negative geotaxis phenotype in Cyfip85.1/+ flies. Negative geotaxis response was measured in 5 days old Cyfip85.1/+ and control male (left) and female (right) flies. n>30 flies for each genotype, mean±s.e.m. ns, not significant, Mann-Whitney test.

FIG. 2. Analysis of the Cyfip haploinsufficiency proteome revealed maladaptive mitochondrial function and energy production and a link to neurological disorders. Top: Gene ontology cellular component analysis. Middle: Gene ontology biological functions analysis of Cyfip^{85.1}/+ flies compared to controls. Enrichment values were calculated using the Topology-based Elimination Fisher method. Bottom: Disease enrichment values for the deregulated genes of Cyfip^{85.1}/+ flies compared to controls using combined OMIM/geneRIF/Ensembl variation annotation data. Enrichment values were calculated using the Topology-based Elimination Fisher method; N=number of disease genes found in the dataset; p-values making use of the full HDO ontology tree (6331 terms).

FIG. 3. Increased brain mitochondrial activity and mitochondrial membrane potential in Cyfip haploinsufficient flies. (A) Quantification of the oxygen consumption normalized per unit (1 unit corresponding to 1 brain) of control and Cyfip^{85.1}/+. n≥6 independent experiments per genotype (each with 10 fly brains), mean±s.e.m. **p<0.01, multiple t test, corrected for multiple comparisons using the Sidak-Bonferroni method. (B) Left: Representative confocal images of fly brains from control and Cyfip^{85.1}/+ stained with Rhodamine 123. Right: Quantification of fold change in Rhodamine 123 fluorescence intensity from the antenna lobes (AL) area. n=11-12 brains per genotype, mean±s.e.m., *p=0.0167, Kruskal-Wallis test, Dunn's multiple comparison test. (C) Measurement of the mitochondrial membrane potential. Left: representative Z projections of confocal images of fly brains stained with TMRE. The antenna lobes (AL) area was quantified (highlighted with a circle). Right: quantification of the TMRE staining from control and Cyfip^{85.1}/+. Shown is the fold change in TMRE fluorescence intensity from the AL area. n=15-16 brains per genotype, mean±s.e.m., **p<0.01, Mann-Whitney test. (D) Left: Representative EM images of mitochondria from control and Cyfip^{85.1}/+ fly brains. Right: Quantification of the mitochondria perimeter and area. n=160-165 mitochondria per genotype, mean±s.e.m. ****p<0.0001, Mann-Whitney test. (E) Left: Food consumption during the day time. Groups of 5-7-day-old flies were analyzed every 2 hours (ZT0, ZT2, ZT4, ZT6). Right: Quantification of food consumption over 24 hours. At the given timepoints, flies were collected, homogenized, and the absorbance of the supernatant measured at 625 nm (to read the blue dye in the food). mean±s.e.m., ns, not significant, Mann-Whitney test.

FIG. 4. Altered TCA cycle and metabolism in Cyfip haploinsufficient flies. (A) Simplified representation of TCA cycle and metabolites measured in fly brains. (B) Quantification of metabolites from the TCA cycle in Cyfip^{85.1}/+ brains normalized to control. n=5 (group of 10 fly brains), mean±s.e.m. *p<0.05, **p<0.01, ***p<0.001, Kruskal-Wallis test, Dunn's multiple comparison test. (C) Left: Enzymatic activity of IDH-NAD dependent from Cyfip^{85.1}/+ brains normalized to control. n=3-4 (group of 10 fly brains), mean±s.e.m. **p<0.01, Mann-Whitney test. Right: same as in the left panel, but for the αKG enzymatic activity. n=3-4 (group of 10 fly brains), mean±s.e.m. *p<0.05, Mann-Whitney test. (D) Relative levels of NADH of fly brains from control and Cyfip^{85.1}/+. n=4 (group of 10 fly brains), mean±s.e.m. *p<0.05, Mann-Whitney test. (E) Contribution of [U-¹³C₆]glucose to TCA-cycle metabolites in Drosophila brain tissue following 6 hours feeding with [¹³C]glucose. The x-axis denotes the ¹³C-isotopologue profile (i.e. the mass isotopologue distribution—MID) of labeled metabolites following the incorporation of [¹³C] glucose skeleton. The catabolism of ¹³C labeled glucose leads to M+3 pyruvate. The decarboxylation of pyruvate makes M+2 acetyl-CoA in the mitochondria, which is then incorporated into M+2 citrate. All TCA cycle intermediates, downstream of citrate, will have a mass of M+2. Values shown are mean±sem. n=5 (10 brains were pooled per each sample). ***p<0.001, **p<0.01, *p<0.05, Sidak's multiple comparisons test.

FIG. 5. TCA cycle and mitochondrial complex I inhibition restores social deficits but not hyperactivity of Cyfip mutant flies. (A) Social behavior of Cyfip^{85.1}/+ upon genetic reduction of idh3a, n>10 groups of 8 flies for each genotype were analyzed, **p<0.01, ***p<0.001, Kruskal-Wallis test, Dunn's multiple comparison test. (B) Social behavior upon treatment with M of ML309 for 1 (1d) or 3 days (3d) of Cyfip^{85.1}/+ flies (left) and upon pan-neuronal IDH overexpression (29° C.) in wild-type (WT) adults (right). n>10 independent experiments (each with 8 flies) for each genotype, mean±s.e.m., **p<0.01, ***p<0.001, ****p<0.0001, Kruskal-Wallis test, Dunn's multiple comparison test. (C) Social behavior upon feeding with M rotenone for 1 (1d), 2 (2d) and 4 days (4d) in Cyfip^{85.1}/+ flies, n>10 groups of 8 flies for each genotype were analyzed, ****p<0.0001, Kruskal-Wallis test, Dunn's multiple comparison test. (D) Social behavior upon treatment with different concentrations of Rotenone, in control flies. n>10 independent experiments (group of 8 flies) for each genotype, mean±s.e.m., Kruskal-Wallis test, Dunn's multiple comparison test. (E) The positive effect of rotenone treatment is reversible in Cyfip^{85.1}/+ flies. Cyfip mutant flies were treated for 4 days (4d) with M rotenone and then tested for 5 days of the post-treatment period in competition for food assay. The comparison was with the control in each case. n>10 groups of 8 flies for each condition were tested. mean±s.e.m., ****p<0.0001, **p<0.01, Kruskal-Wallis test, Dunn's multiple comparison test. (F) Competition for food in Cyfip^{85.1}/+ flies or upon genetic reduction of pink1 or ndufs3, n>10 groups of 8 flies for each genotype were analyzed, ****p<0.0001, Kruskal-Wallis test, Dunn's multiple comparison test. (G) Left: Representative Western blotting of protein extracts from fly brains using antibodies to detect the mitochondrial proteins HSP60, ATPB, NDUFS3 and TOM20. Right: quantification upon normalization to Syntaxin, n=4 per genotype, mean±s.e.m *p<0.05. Kruskal-Wallis test, Dunn's multiple comparison test. (H) Rotenone does not rescue hyperactivity in Cyfip^{85.1}/+ flies. Locomotion upon treatment with 10 μM rotenone for 4 days (4d) was analyzed in Cyfip^{85.1}/+ flies. Locomotion was monitored over 24 hours. n>40 flies for each condition were tested. mean±s.e.m., **p<0.01, Kruskal-Wallis test, Dunn's multiple comparison test.

FIG. 6. The social deficits of Cyfip haploinsufficiency flies are due to defects in the GABAergic circuitry. (A) Abrogation of Cyfip in Cholinergic and (B) GABAergic neurons. Flies were analyzed for total number of social interactions. n=14 independent experiments with 8 flies per genotype. mean±s.e.m, ****p<0.0001, ***p<0.001, **p<0.01, Kruskal-Wallis test, Dunn's multiple comparison test. (C) Cyfip mRNA levels upon Cyfip knockdown in GABAergic neurons (FACS-sorted cells) (white bar) in comparison to control flies (black bar). The levels of Cyfip were normalized to rpl13 mRNA. n=3 for each genotype, mean±s.e.m. **p<0.01, Mann-Whitney test.

FIG. 7. Flies with Cyfip haploinsufficiency have impaired GABA signaling. (A) Left: representative confocal images of control and Cyfip^{85.1}/+ and Cyfip^{85.1}/+ fed flies for 5 days (5d) with 50 μM GABA and stained with GABA. Right: Data quantification (n=15 brains). Scale bars, 100 m. ****p<0.0001, Kruskal-Wallis test, Dunn's multiple comparison test. (B) GABA levels determined by HILIC-MS/MS in control and Cyfip^{85.1}/+ brains. n=5 (group of 10 brains) per genotype, mean±s.e.m, Mann-Whitney test. (C) Analysis of GABA levels from control and Cyfip^{85.1}/+, into enriched fractions of mitochondria and vesicles by ELISA. n>3, mean±s.e.m. **p<0.01, Kruskal-Wallis test, Dunn's multiple comparison test. (D) Subcellular fractionation and enrichment in mitochondria and synaptosomes. Shown is a Western blot of the respective fractions. Bruchpilot (BRP) was used as synaptic marker and ATP5B was used as mitochondrial marker.

FIG. 8. Increasing GABA levels restores social deficits of Cyfip haploinsufficient flies. (A-C) Social events of Cyfip^{85.1}/+ flies upon treatment (2-5 days) with 50 μM GABA, 100 μM DABA, 1 mM VPA. (D) Social events of Cyfip^{85.1}/+ flies upon treatment (1 d) with 50 μM Diazepam. (E) Social events upon treatment with VPA, GABA, DABA, in flies with reduced CYFIP in GABAergic neurons. For all the experiments n>10 (group of 8 flies), **p<0.01, ***p<0.001, ****p<0.0001, Kruskal-Wallis test, Dunn's multiple comparison test. (F) Conditional abrogation (ElavGal4;TubGal80^{ts}>UAS-CYFIP-IR 29° C.+ GABA) of Cyfip pan-neuronally in adults upon GABA treatment for 5 days in comparison with the uninduced (Cyfip not reduced; ElavGal4;TubGal80^{ts}>UAS-CYFIP-IR 18° C.) and induced (Cyfip reduced; ElavGal4;TubGal80^{s}>UAS-CYFIP-IR 29° C.) controls. Flies were tested for social interactions. n>14 independent experiments (group of 8 flies) for each genotype, mean±s.e.m., ***p<0.001, Kruskal Wallis test, Dunn's multiple comparison test. (G) Social behavior upon GAD1 abrogation pan-neuronally in adults. n>10 independent experiments (each with 8 flies) for each genotype, mean±s.e.m., ***p<0.001, Kruskal-Wallis test, Dunn's multiple comparison test. (H) Social activity of control flies is monitored upon treatment with GABA for 5 days (5d). n>10 independent experiments (each with 8 flies) for each genotype, mean±s.e.m., *p<0.05, Mann-Whitney test. (I) Temporal effect of GABA treatment in competition for food. Cyfip⁸⁵-i+ flies were treated for 5 days with GABA (GABA 5d) and then tested for 5 days of the posttreatment period. The comparison was with the control in each case. n>10 groups of 8 flies for each condition were tested. mean±s.e.m., ****p<0.0001, **p<0.01, Kruskal-Wallis test, Dunn's multiple comparison test.

FIG. 9. Aralar is a novel GABA transporter which regulates behavior. (A) Social behavior (in food competition assay) genetic screening for control, Cyfip^{85.1}/+ and mitochondrial carrier/transporter mutant flies crossed with Cyfip^{85.1}/+ mutant flies. Threshold line indicates the control levels of behavior. # shows the genotype that is significantly different from Cyfip^{85.1}/+ and not different from the control. mean±s.e.m, n>10 groups of 8 flies, Kruskal-Wallis test, Dunn's multiple comparison test. (B) Analysis of GABA levels by ELISA in cytoplasmic and mitochondrial fractions from control, Cyfip^{85.1}/+, Aralar^{MI07552}/+ and Aralar^{MI07552}/Cyfip^{85.1} flies. n>3, mean±s.e.m. **p<0.01, *p<0.05, Kruskal-Wallis test, Dunn's multiple comparison test. (C) Social behavior (in food competition assay) in control, Cyfip^{85.1}/+, Aralar^{MI07552}/+ and Aralar^{MI07552}/Cyfip^{85.1} flies. n>8 independent experiments (group of 8 flies) for each genotype, mean±s.e.m., ***p<0.001, **p<0.01, *p<0.05, Kruskal-Wallis test, Dunn's multiple comparison test. (D) Grooming behavior in control, Cyfip^{85.1}/+, Aralar^{MI07552}/+ and Aralar^{MI07552}/Cyfip^{85.1} flies. n>8 independent for each genotype, mean±s.e.m., ***p<0.001, **p<0.01, *p<0.05, Kruskal-Wallis test, Dunn's multiple comparison test. (E) Social space behavior in control, Cyfip^{85.1}/+, and Aralar^{MI07552}/Cyfip^{85.1} flies. n>8 independent experiments (group of 30 flies) for each genotype, mean±s.e.m., ***p<0.001, **p<0.01, *p<0.05, Kruskal-Wallis test, Dunn's multiple comparison test. (F) Aralar haploinsufficiency does not rescue hyperactivity deficits of Cyfip mutant flies. Locomotor activity was monitored over 24 hours. n>40 flies for each genotype were tested. mean±s.e.m., **p<0.01, ***p<0.001, Kruskal-Wallis test, Dunn's multiple comparison test.

FIG. 10. Treatment with an Aralar inhibitor rescues the deficits in social behavior of Cyfip^{85.1}/+ mutant flies. Social behavior (in food competition assay) of control flies or Cyfip^{85.1}/+ flies upon treatment with 1 mM of pyridoxal 5′-phosphate hydrate for 1 day or 2 days. n>8 independent experiments (each with 8 flies) for each genotype, mean±s.e.m., **p<0.01, ***n<0.001, ****n<0.0001, Kruskal-Wallis test, Dunn's multiple comparison test.

FIG. 11. Aralar expression is not different in the Cyfip^{85.1}/+ flies but the proton gradient across the mitochondrial membrane drives the GABA-transporting activity of Aralar. (A) Aralar mRNA level was measured in control and Cyfip^{85.1}/+ fly brains by RT-qPCR and quantified upon normalization to the ribosomal protein rpl13 mRNA. n=4 (groups of 10 fly brains each), mean±s.e.m, Mann-Whitney test. (B) Translational efficiency of Aralar mRNA is comparable in control and Cyfip^{85.1}/+ flies. Distribution of Aralar mRNA on polysomes and mRNPs. Bars indicate the percentage Aralar mRNA on polysomes over the total (polysomes+ mRNPs). n=3 groups of 100 fly heads for each genotype, mean±s.e.m. Mann-Whitney test. (C) Upon treatment with 500 μM oligomycin, mitochondrial membrane potential was analyzed by staining using TMRE, n=8-9 brains. (D) Competition for food of control flies upon treatment (1 day) with 500 μM of oligomycin, n=10 groups of 8 flies each. (E) GABA levels measured by ELISA in fractionated mitochondria. n=5, mean±s.e.m., ***p<0.001, *p<0.05, Kruskal-Wallis test.

FIG. 12. Model: Aralar drives GABA into polarized mitochondria. In the absence of Cyfip, TCA cycle produces increased levels of succinate and NADH leading to higher mitochondrial activity that next leads to hyperpolarization of mitochondria. As a consequence, the increased polarization activates the GABA carrier Aralar driving GABA into mitochondria. The impaired unidirectional GABA transport into the mitochondria reduces its homeostatic levels required for proper synapse functioning and behavior.

FIG. 13. Reduced GABA levels in neurons and urine of Cyfip1^{+/-} mice. Cyfip haploinsufficiency shows impaired GABA signaling in mice. (A) Left: WT and Cyfip1^{+/-} cortical neurons were stained with a GABA antibody. Right: Data quantification (n=3 mice per genotype, >100 cells in total), mean±s.e.m., Scale bars, 30 m. ****p<0.0001, Kruskal-Wallis test. (B) GABA levels determined in urine of WT and Cyfip1^{+/-} mice, n=8 per genotype, mean±s.e.m, **p<0.01, Mann-Whitney test.

FIG. 14. Diazepam restores deficits in social behavior in Cyfip1 haploinsufficient (Cyfip1+/− mice. (A) Left: schematic representation of the 3-chamber protocol used for assessing social preference (sociability). Right: Sociability index (stranger 1 vs object) in the three-chamber test in WT and Cyfip1+/− mice. N=9 mice for each genotype, mean±s.e.m., p=0.0088, unpaired t-test. (B) Left: schematic representation of the 3-chamber protocol for social novelty. Social novelty index (stranger 1 vs stranger 2) in WT and Cyfip1^{+/-} mice. N=9 mice for each genotype, mean±s.e.m., p=0.3901, unpaired t-test. (C) Left: sociability index (stranger1 vs object) in the three-chamber test in WT and Cyfip1+/− mice during control and diazepam conditions. n=9 for each genotype, mean±s.e.m, p=0.0261 genotype-treatment significant interaction effect, two-way ANOVA. Right: changes of sociability index upon acute diazepam treatment within-animal (p=0.0135, mean±s.e.m., two-tailed t-test). (D) Left: social novelty index (stranger 1 vs stranger 2) in the three-chamber test in WT and Cyfip1+/− mice during control and diazepam conditions. n=9 for each genotype mean±s.e.m., p>0.05 for all effects, two-way ANOVA. Right: within-animal changes of social novelty index upon acute diazepam treatment (p>0.05, mean±s.e.m., two-tailed t-test).

FIG. 15. Fmr1 mutant flies show deficits in social behavior. Competition for food, in control, Fmr1D50/+, Aralar^{MI07552}/+ and Aralar^{MI07552}/Fmr1D50 flies. n=8 independent experiments with groups of 8 flies. All comparisons refer to the control. ***p<0.001, **p<0.01, Kruskal-Wallis test, Dunn's multiple comparison test.

FIG. 16. Drug screening to identify compounds that ameliorate social behavior deficits in flies with Cyfip haploinsufficiency. (A) Control (w1118) and CYFIP (Cyfip^{85.1/+}) flies treated with various compounds from the Prestwick library were analyzed for the total number of social interactions in a competition for food assay (n>6 groups of 8 flies for each compound). The Figure is split across two pages. All legends relate to both parts. On the x-axis: Control+ veh=w1118 flies without test compound; CYFIP+ veh=Cyfip^{85.1/+} flies without test compound; Various identifiers=Cyfip^{85.1/+} flies+ certain Prestwick compound as described herein (batho: bathophenanthroline). On the y-axis: Total number of social interactions is shown as a shade of grey according to the scale at days 1, 2 and 5 of treatment with the compound or vehicle. Bottom row (A: 1 day), middle row (B: 2 days), upper row (C: 5 days). Hit compounds that increased the number of social interactions in Cyfip^{85.1/+} flies are marked with boxes.

### EXAMPLES

Methods and materials are described herein for use in the present disclosure; other, suitable methods and materials known in the art can also be used. The materials, methods, and examples are illustrative only and not intended to be limiting.

Example 1. Drosophila stocks and rearing conditions. Flies were kept in vials containing a standard Drosophila medium at 25° C. with 60-80% humidity in a 12h light/dark cycle. The fly line used as control was wild-type Canton-S w¹¹¹⁸ (isolCJ). Heterozygous mutant flies for Cyfip (Cyfip^{85.1}/+) lacking two-third of the Cyfip-coding region were described previously by Schenck (2003), Neuron 38(6):887-98. The Cyfip^{85.1}/+ mutant flies used in this study were first isogenized for 6 generations with a Cantonized w¹¹¹⁸ background.

To control Cyfip expression, the tissue specific GAL4/UAS system was employed (Brand and Perrimon, 1993). In brief, GAL4 binds to UAS which activates transcription of a RNAi construct (Cyfip-IR) that interferes with the endogenous Cyfip transcript which turn is downregulated. Specifically, the UAS-Cyfip-RNAi transgene (UAS-Cyfip-IR) was used to abrogate Cyfip expression as described in Galy et al., 2011. To generate GAL4/UAS-Cyfip-IR flies, GAL4/GAL4 females were crossed en masse to UAS-Cyfip-IR/UAS-Cyfip-IR males. The GAL4 drivers are described in Kanellopoulos (2012), J Neurosci. 32(38):13111-24. GAL4 and UAS lines have been isogenized for 6 generations with the w1118 background before crossing. GAL4/GAL4 and UAS-Cyfip-IR/UAS-Cyfip-IR females were also crossed with w¹¹¹⁸ males to generate GAL4/+ and UAS-Cyfip-IR/+ progeny respectively that served as controls which should not produce the RNAi construct.

Tissue-specific GAL4 expression was achieved using the tissue-specific GAL4 promoters as follows: pan-neuronal (Elav), in cholinergic (Cha-Gla4), and GABAergic (Gad-Gal4). To avoid neurodevelopmental problems and possible lethality, the ElavGal4, was combined with the temporal TubGal80^{ts} system (McGuire et al., 2003; McGuire et al., 2004). At 18° C., TubGal80^{ts} is expressed and binds GAL4, prohibiting its binding to the UAS (i.e., no transcription of UAS-Cyfip-IR). In contrast, at 29-30° C. TubGal80^{ts} is degraded and will release GAL4 which in turn will bind the UAS thus activating transcription of UAS-Cyfip-IR. As a result, expression of Cyfip will be downregulated. All flies/progeny for promoter-Gal4;TubGal80^{ts} and their controls were cultured at 18° C. and 2-days after eclosion were placed at 29-30° C. allowing UAS-Cyfip-IR transcription.

All the behavioral experiments with flies were performed in a walk-in chamber by Fitoclima Aralab that offers highly precise and reproducible conditions for climatic and temperature testing.

Example 2. Assays to test the behavior of Drosophila flies. To determine the behavior of the flies, a food competition assay (social events), a pair mating assay, a grooming assay, and a social space assay were employed. Furthermore, locomotor activity tests and a negative geotaxis test were performed to control for potential deficits of the motor system. Drosophila quantitative assay for social events (competition for food).

Experiments were performed with socially experienced, 3-7 days-old male flies. Groups of eight males from the same genotype were anesthetized 24 h prior to the assay and placed in vials with food. On the day of the assay, the males were transferred without anesthesia to an empty vial and were deprived of food for 90 minutes, after which they were exposed to a food droplet and given 2 minutes to acclimate to this disturbance. The flies were then observed simultaneously for an additional 2 minutes, and the total number of social events was scored. Monitored social events were i.e. approaches, lunges, tussles, wing threat and the initiation of courtship. Of note, one interaction involving two or more flies was counted as one social event in total (Zwarts (2011), PNAS 108, 17070-17075. The behavioral assays were conducted in a behavioral chamber (25° C., 60-70% humidity) between 8 a.m. and 11 a.m. Statistics were calculated using a one-way fixed effect ANOVA model, and Kruskal Wallis test in combination with Dunn's multiple comparison test.

### Drosophila Single Pair Mating Assay (Courtship Index).

The social performance of flies was further investigated by monitoring courtship, a complex innate Drosophila behavior. Briefly, courting Drosophila males perform a characteristic sequence of behaviors: orienting toward and following the female, tapping her with their forelegs, vibrating one wing, licking her genitalia, and attempting to copulate (Bastock (1956), Evolution 10, 421-439; Bastock (1955), Behaviour 8, 85-111; Ejima (2007), CSH Protoc. 2007:pdb.prot4847; Sokolowski (2001), Nat Rev Genet 2, 879-890; Sokolowski (2010), Neuron 65, 780-794).. Five to seven days old virgin males and females were used. Each assay consisted of one Cyfip^{85.1}/+ or wild type (control) male and one wild type female introduced into a plexiglass-mating chamber (1 cm diameter×4 mm height). The courtship activities were video recorded until successful copulation, or longer (6 minutes) in the absence of copulation, using a 65×SD camcorder (Samsung) mounted on a tripod. The assays were done under ambient light at 22° C. temperature and 70% humidity. From the video recordings the courtship index (CI) was calculated for each male. Briefly, the CI is the fraction of total recording time where the male showed courtship behavior (orienting, chasing, tapping, licking, singing, copulation attempts) (Ejima (2007), CSH Protoc. 2007:pdb.prot4847; Siegel (1979), PNAS 76, 3430-3434; Sokolowski (2001), Nat Rev Genet 2, 879-890; Sokolowski (2010), Neuron 65, 780-794).

### Drosophila Grooming Behavior

5 days old flies were collected the day before the assay and kept in vials with fresh food. The day of the experiment they were anesthetized by placing them on ice for 2 min and then placed into a grooming chamber (circular arena of 1 cm diameter). Flies were allowed for 20 min to acclimate and then grooming activity was recorded for 3 min, using a 65×SD camcorder (Samsung) mounted on a tripod. To measure grooming behavior, raw videos were analysed, wherein the researcher were blinded to the genotype, and the percentage of grooming time over 3 min was calculated.

### Social Space Behavior

Flies were separated by gender the day prior to each experiment and kept in vials with fresh food. The analysis of social space behavior was performed using a horizontal circular chamber (a Petri dish of 9 cm diameter) as described before (Simon (2012), Genes Brain Behav 11, 243-252). Flies were briefly anesthetized by placing them on ice for 2 min and placed into the chamber. Flies were let to acclimate for 10 min and then digital images were collected after the flies reached a stable position (up to 25 min). Digital images were imported in Image J and an automated measure of the nearest neighbor to each fly was determined using an R script. Due to thigmotaxis behavior, flies tend to move closer to the wall of an open arena to avoid the more threatening central part (centrophobicity) (Besson (2005), J Neurobiol 62, 386-396). To further asses the thigmotaxis behavior, the distance of a fly from the wall of the chamber was measured using an R script. Graphs and statistical analysis were performed using Prism 4 (GraphPad Software, San Diego, CA, USA).

### Negative Geotaxis Experiment.

The negative geotaxis experiment allows to evaluate the motor reflex response in Drosophila as previously described (Kosmidis (2011), Neurobiol Dis 43, 213-219). Briefly, 2-days-old control and Cyfip^{85.1}/+ flies were transferred individually into polystyrene tubes. The flies were allowed to acclimate for 10 min in a dark environment of 25° C. and 70-80% humidity illuminated by red light. Each fly was vortexed for 2 sec and tested twice for negative geotaxis measuring the time the fly climbs the distance of 6 cm.

### Drosophila Locomotor Activity.

The Drosophila Activity Monitoring (DAM) system from Trikinetics Inc. (Walthman, MA) was used to record the locomotor activity of flies. Male flies (1-week old), anesthetised on ice, were individually loaded into the locomotor activity-monitoring tubes, thin 5 mm diameter polycarbonate tubes containing 5% sucrose and 2% agar food and incubated for at least 4 days under 12:12 LD (light-dark) conditions at 25° C. and 50-60% humidity. Light was turned on at 7:30 am and turned off at 7:30 pm. The DAM system software counts the number of times the single fly walks through an infrared beam aimed at the middle of the tube. Data were recorded as the number of crossings (transitions) per bin of 5-30 minutes. To allow the flies to recover from anaesthesia and to get acclimatized to the new environment data from the first day was excluded from the analysis. Two independent experiments were performed and at least 40 flies for each genotype were analysed. Statistical analysis was conducted using GraphPad Prism 6.0 software. Data were evaluated by one-way analysis of variance (ANOVA) with Sidak's post hoc test and by non-parametric Kruskal-Wallis's test with Dunn's correction.

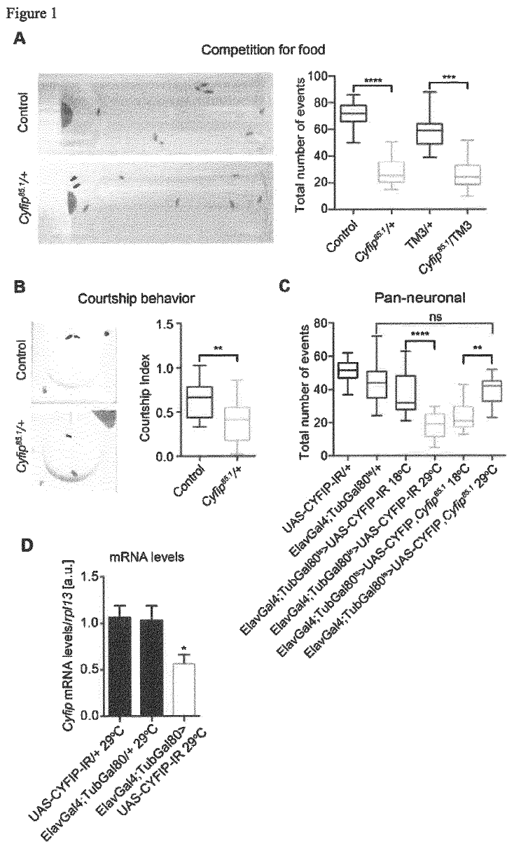

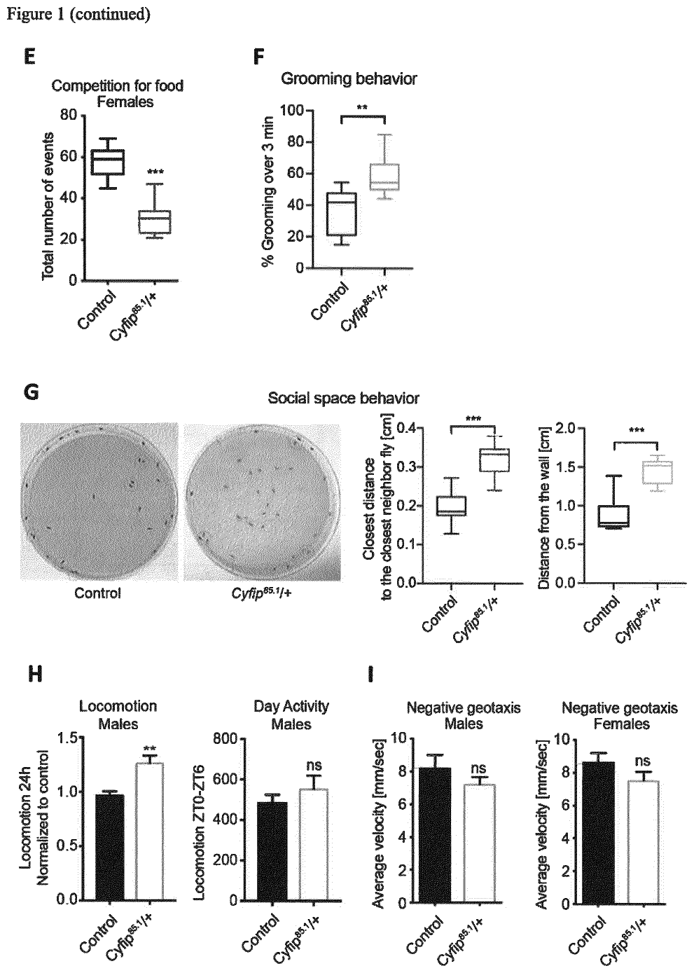

Example 3. Reduced Cyfip levels lead to deficits in behavior. First, male flies or female were tested in a food competition assay as described in Example 2 to determine the total number of social events. Both, male and female Cyfip^{85.1}/+ flies showed a reduced number of social events relative to control flies (FIGS. 1A and 1E). Second, the social performance of the Cyfip^{85.1}/+ flies was further investigated by observing Drosophila courtship, a complex innate social behavior in flies, as described in Example 2. Cyfip^{85.1}/+ male flies showed a decreased courtship index towards a wild type female (FIG. 1B). Third, the grooming behavior of individual flies was monitored in an observation chamber (Tauber (2011) PloS one 6, e27100) as described in Example 2. Cyfip^{85.1}/+ male flies showed an excess of grooming behavior in comparison to control flies (FIG. 1F) suggesting an increase in repetitive behavior. Furthermore, the behavior of flies within a social group was recorded by measuring the distance between two flies as social space as described in Example 2 (Simon (2012), Genes Brain Behav 11, 243-252). Cyfip^{85.1}/+ flies, on average, showed a longer distance to the closest neighbor fly in comparison to control flies within a group (FIG. 1G) confirming further the social interaction impairment of Cyfip^{85.1}/+ flies. In addition, Cyfip^{85.1}/+ flies stayed further away from the wall of the open field arena (FIG. 1G), indicating that their natural thigmotactic behavior (attraction to the contact of the wall) (Besson (2005), J Neurobiol 62, 386-396) was also disturbed. Cyfip^{85.1}/+ flies exhibited hyperactivity (assayed as described in Example 2) overs 24 hours when compared to controls (FIG. 1I, left), but no differences in locomotion activity were observed during the day time which was the time when the behavioral assays were performed (FIG. 1I, right). This indicates that hyperactivity did not interfere with the behavioral assays and the observed behavioral deficits were not due to impaired motor ability. Furthermore, Cyfip mutant flies (males and females) did not differ from the controls (Figure S1D and E) in negative geotaxis which is a measurement for motor reflex as described in Example 2 (Sun (2018), Front Syst Neurosci 12, 6). In addition, it has been previously shown that Cyfip mutants have normal anxiety (Garcia (2019), J Exp Zool A Ecol Integr Physiol 331, 192-200) and startle response (Zhang (2002), Genetics 162, 1283-1299 behavior.

Then, the social behavior was assayed upon temporal and regional specific dCyfip knock-down in adult flies by using UAS-Cyfip-IR transgenic flies as described in Example 1. Pan-neuronal attenuation of Cyfip mRNA levels in adult flies (FIG. 1D) phenocopied the decreased competition for food in Cyfip^{85.1}/+ mutant animals (FIG. 1C). The decreased social score in the Cyfip^{85.1}/+ flies was fully rescued by expression, in adulthood only, of a transgene harboring the wild type Cyfip (UAS-Cyfip-WT/genetic rescue) (FIG. 1C). These data provide evidence that Cyfip is required for normal behavior in adult flies.

Example 4. Proteomic and metabolic analyses of Cyfip^{85.1}/+ mutant flies link mitochondrial processes with neurological disorders. To identify the molecular mechanism underlying the observed behavioral dysfunctions in the Cyfip^{85.1}/+ flies, tandem mass spectrometry was performed in control and Cyfip^{85.1}/+ mutant brains and a total of 345 dysregulated proteins was identified. In brief, sample was dissolved in Laemmli buffer and run on a 10% SDS-PAGE gel, which was stopped when the front reached about ⅕ of the gel. The gel was fixed overnight and stained briefly with colloidal coomassie blue. The protein-containing gel piece was chopped into 1 mm by 1 mm pieces, destained, and subjected to trypsin digestion as described previously (Chen (2015), Biochim Biophys Acta 1854, 827-833). The tryptic peptides were dissolved in 17 μL 0.1M acetic acid, and analyzed by nano-LC MS/MS using an Ultimate 3000 LC system (Dionex, Thermo Scientific) coupled to a TripleTOF 5600 mass spectrometer (Sciex). Peptides were fractionated on a 200 mm Alltima C18 column (100 μm i.d., 3 μm particle size). The acetonitrile concentration in the mobile phase was increased from 5 to 30% in 90 minutes, to 40% in 5 minutes, and to 90% in another 5 minutes, at a flow rate of 400 nL/minutes. The eluted peptides were electro-sprayed into the TripleTOF MS. The nano-spray needle voltage was set to 2500V. The mass spectrometer was operated in a data-dependent mode with a single MS full scan (m/z 350-1200, 250 msec) followed by a top 25 MS/MS (85 msec per MS/MS, precursor ion>90 counts/s, charge state from +2 to +5) with an exclusion time of 16 sec once the peptide was fragmented. Ions were fragmented in the collision cell using rolling collision energy, and a spread energy of 10 eV. The MS raw data were imported into MaxQuant (version 1.5.2.8), and searched against the uniprot-proteome_fruitfly_%3AUP000000803 database, with match between run enabled. Further MaxQuant settings were left at default.

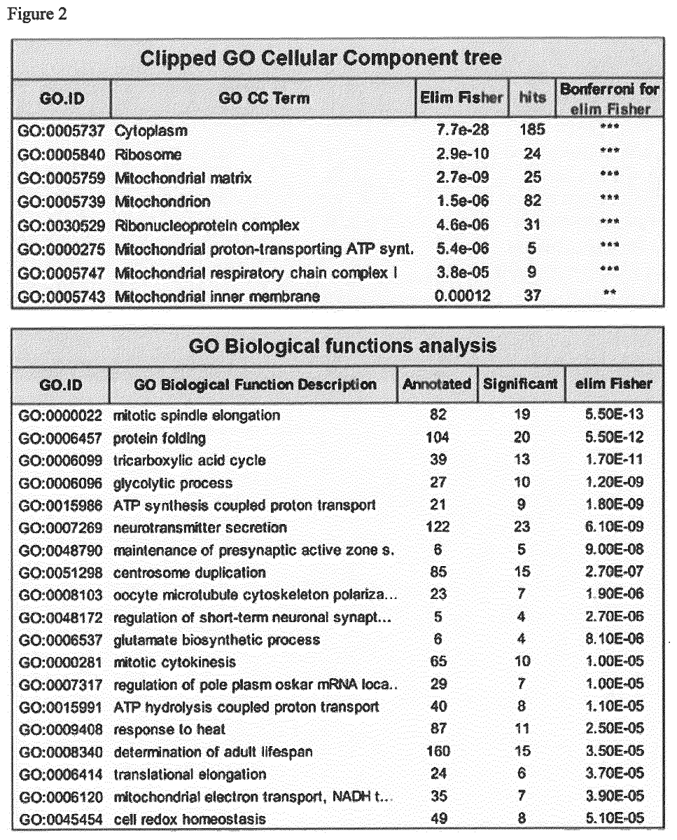

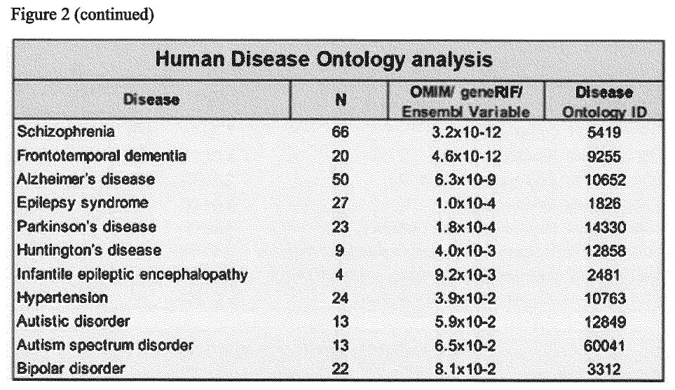

Gene ontology analysis revealed a strong implication of mitochondria (FIG. 2) and mitochondrial processes such as the dysregulation of the TCA cycle, energy metabolism and mitochondrial electron transport, as well as neurotransmitter release related processes (FIG. 2C). Furthermore, disease-related analyses identified a highly significant overrepresentation of gene networks implicated in neurological diseases such as schizophrenia, dementia, and syndromic epilepsy (FIG. 2). Disease enrichment of the union dataset (mapped to 278 human genes using the DRSC DIOPT tool (Hu (2011), BMC Bioinformatics 12, 357) was performed using the gene-disease annotation data collected from OMIM (Amberger (2015), Nucleic Acids Res 43, D789-798), GeneRIF (Mitchell (2003), AMIA Annu Symp Proc 460-464) and Ensembl variation (Chen (2010), BMC Genomics 11, 293) databases using the topOnto package (https://github.com/statbio/topOnto). The annotation data was standardized using MetaMap (Aronson (2010), J Am Med Inform Assoc 17, 229-236) and NCBO Annotator to recognize terms found in the Human Disease Ontology (HDO) (Schriml (2012), Nucleic Acids Res 40, D940-946). Recognized disease ontology terms were then associated with gene identifiers and stored locally. Disease term enrichment, for the 279 human genes, were then be calculated using the Topology-based Elimination Fisher method (Alexa (2006), Bioinformatics 22, 1600-1607) found in the topGO package (http://topgo.bioinf-mpi-inf.mpg.de/), together with the standardized OMIM/GeneRIF/Ensembl variation gene-disease annotation data (17731 gene-disease associations), and the full HDO tree (3140 terms). The dataset was tested for functional enrichment using the GO Cellular Component (CC) and Biological Function (BP) ontologies (Ashburner (2000), Nat Genet 25, 25-29), together with the topGO package and Elimination Fisher method. 1078 (1692) CC terms were found for Fly (Human) and 6479 (14291) BP terms. Enrichment results are shown in FIG. 2, where P-values have been corrected using the Bonferroni correction at the 0.01 (***) and 0.05 (**) significance level, using the number of terms in the GO tree.

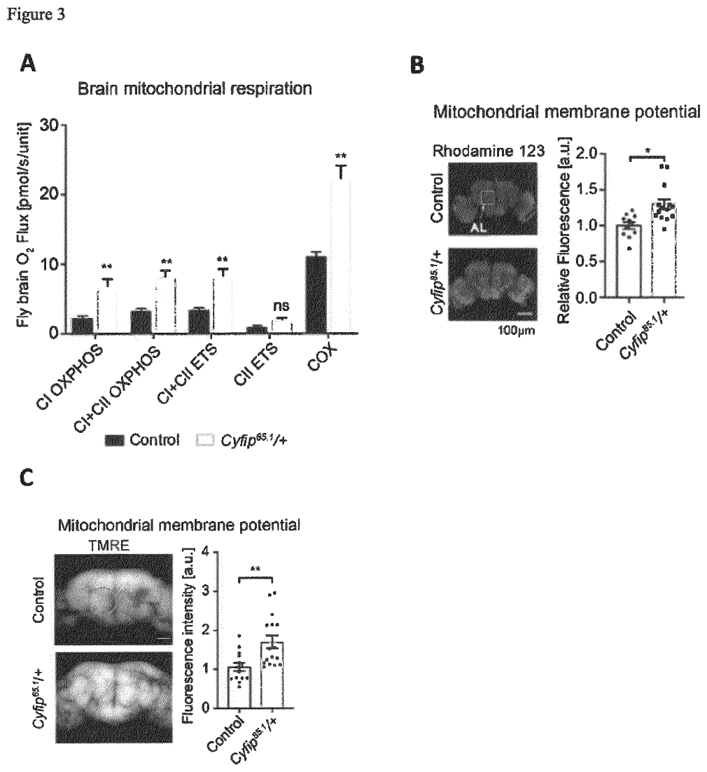

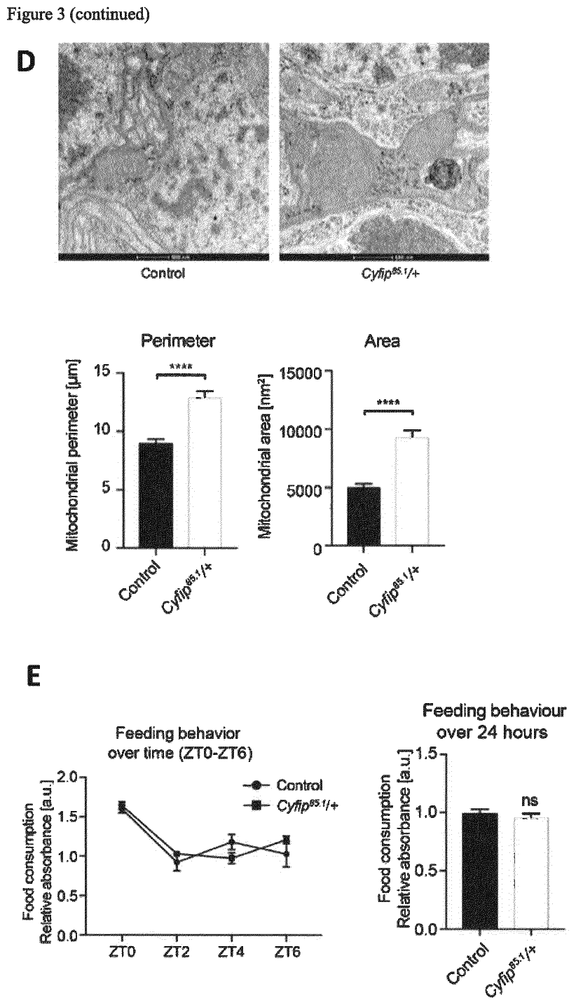

Next, mitochondrial processes were analyzed in more detail. Using high-resolution respirometry, a striking increase in mitochondrial respiratory capacity through the respiratory chain in Cyfip^{85.1}/+ brains was observed (FIG. 3A). In brief, ten fly heads from 4 days old male were rapidly dissected under a microscope and mechanically homogenized in Miro6 Buffer (20 mM Hepes, 110 mM sucrose, 10 mM KH₂PO4, 20 mM taurine, 60 mM lactobionic acid, 3 mM MgCl₂, 0.5 EGTA, pH 7.1, 1 mg/ml fatty acid free BSA, catalase 280 U/ml) (Krumschnabel et al., 2015), then immediately loaded into an Oroboros 2K oxygraph chamber filled with Miro6 buffer equilibrated at 25° C. The intactness of the mitochondrial outer membrane was checked by addition of exogenous cytochrome c. Oxygen consumption rates were measured before and after addition of the following sequence of substrates and specific inhibitors: 1) 2.5 mM pyruvate, 1 mM malate (CI leak), followed by 2.5 mM ADP to determine complex I-driven phosphorylating respiration (CI OXPHOS). 2) 5 mM succinate to determine the phosphorylating respiration driven by simultaneous activation of complex I and II (CI+II OXPHOS). 3) Titrating concentrations of the mitochondrial uncoupler CCCP to reach the maximal, uncoupled respiration (CI+II electron transfer system, ETS). 4) 200 nM rotenone to fully inhibit complex I-driven respiration and measure complex II-driven uncoupled respiration (CII electron transfer system, CII ETS). 5) 0.5 μM Antimycin A to block mitochondrial respiration at the level of complex III. Residual oxygen consumption was always negligible. 6) 2 mM ascorbate, 0.5 mM TMPD to measure cytochrome c oxidase (CIV or COX)-driven respiration. 7) 300 μM potassium cyanide to specifically block cytochrome c oxidase activity and measure residual background oxygen consumption caused by chemical interaction between ascorbate and TMPD. Cytochrome c oxidase-driven respiration is the cyanide sensitive oxygen consumption.

Labeling brains with tetramethylrhodamine ethyl ester (TMRE), an established marker for mitochondrial membrane potential (Perry et al., 2011), revealed an increased signal intensity in the Cyfip_{85.1/+} brains, further suggesting that the flies exhibit increased mitochondrial membrane potential (FIG. 3C). Labeling brains with Rhodamine 123, a green-fluorescent dye absorbed by mitochondria as a function of their activity (Perry et al., 2011), also revealed an increased signal intensity in the Cyfip^{85.1}/+ brains, further suggesting that the flies exhibited increased mitochondrial membrane potential (FIG. 3B). Briefly, for the TMRE (Enzo) and Rhodamine 123 (Sigma-Aldrich) stainings, brains were dissected in HL3 solution (mM): 110 NaCl, 5 KCl, 10 NaHCO₃, 5 Hepes, 30 sucrose, 5 trehalose, and 10 MgCl2, pH 7.2. Dissected brains were stained with 10 nM TMRE or Rhodamine123 dye for 5 minutes at room temperature. Brains were quickly washed with HL3 solution, mounted on glass slides and imaged immediately with confocal microscope Leica SP8 using the same settings for all the samples.

Next, mitochondrial morphology was analyzed by Tandem Electron Microscopy (TEM) in fly brain from both genotypes and a remarkable difference was observed in the mutant flies. Precisely, mitochondria area and perimeter were increased in Cyfip^{85.1}/+ mutant flies in comparison to controls (FIG. 3D), which is indicative of higher mitochondrial activity.

In brief, fly brains from 3-5 days old flies were dissected in cold PBS 1× and fixed over-night in 2% paraformaldehyde and 2.5% gluteraldehyde in 0.1 M sodium cacodylate buffer pH 7.4. After rinsing in 0.1 M cacodylate buffer, the samples were post fixed in 1:1 2% OsO4 and 0.2 M cacodylate buffer for 1 hour. After 3 water washes, samples were dehydrated in a graded ethanol series and embedded in an epoxy resin (Sigma-Aldrich). Ultrathin sections (60-70 nm) were obtained with an Ultrotome V (LKB) ultramicrotome, counterstained with uranyl acetate and lead citrate and viewed with a Tecnai G2 (FEI) transmission electron microscope operating at 100 kV. Images were captured with a Veleta (Olympus Soft Imaging System) digital camera. Quantification of mitochondria perimeter and area was performed by using ImageJ software.

The higher metabolic rate of Cyfip^{85.1}/+ flies was not due to higher food consumption, as feeding behavior during the day time and over 24 hours was not altered (FIG. 3E). A colorimetric estimation of food intake was performed as previously described (Aditi (2016), Sci Rep 6, 31411) with some modifications. Briefly, groups of 7-days old mated male flies, entrained for under 12:12 LD (Light-dark cycle), were transferred onto food medium containing 2.5% (w/v) food dye (Erioglaucine blue disodium salt; Sigma-Aldrich), 2% agar and 5% sucrose, and left for feeding ad libitum for 2 hours. After feeding the flies were washed with PBS 1×, each body was detached from the head and singularly homogenized in 100 μl of chilled PBS 1× and centrifuged at 10.000 rpm for 10 min. 50 μl of supernatant was used for absorbance recording at 620 nm using a spectrophotometer.

In conclusion, these data reveal that the mitochondrial activity and size are increased in Cyfip^{85.1}/+ mutant flies.

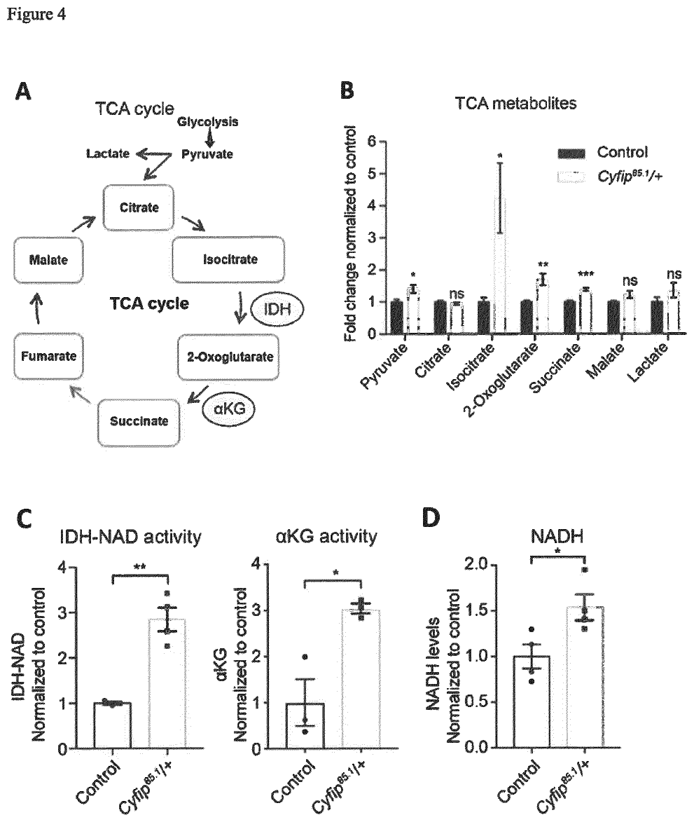

Stable-isotope-dilution Liquid Chromatography Mass Spectrometry (LC-MS) of Cyfip^{85.1}/+ brains revealed that of the metabolites that are produced in the TCA cycle (Krebs cycle/citric acid cycle; FIG. 4A), isocitrate, 2-oxoglutarate (α-ketoglutarate, α-KG) and succinate levels were increased, indicating higher enzymatic activities in this section of the cycle (FIG. 4B). In brief, snap frozen brain samples were pre-extracted and homogenized by the addition of 100 μL of MeOH:H2O (4:1) per 10 fly brains (2×20 seconds at 10000 rpm, Cryolys Precellys Homogenizer, Bertin Technologies, Rockville, MD, US). Homogenized extracts were centrifuged (15 minutes at 4000 g at 4° C.) and the resulting supernatant was evaporated to dryness in a vacuum concentrator (LabConco, Missouri, US). Total protein content was measured in pellets using the BCA assay. Dried sample extracts were resuspended in 50 μL of MeOH:H2O (4:1, v/v) and mixed with 250 μL of the ice-cold internal standard solution (in 100% acetonitrile). Samples were then incubated 30 minutes at −80° C. in order to promote protein precipitation and centrifuged 15 minutes at 4° C. and 2700 g. The resulting supernatants were injected for LC-MS analysis. Quantification of TCA cycle intermediates was performed using Hydrophilic Interaction Liquid Chromatography coupled to tandem mass spectrometry (HILIC-MS/MS) in negative ionization mode using a 6495 Triple Quadrupole system (QqQ) interfaced with 1290 UHPLC system (Agilent Technologies). TCA metabolites were separated using a ZIC-pHILIC (100 mm, 2.1 mm I.D. and 5 μm particle size) column. The mobile phase was composed of A=20 mM ammonium Acetate and 20 mM NH4OH in water at pH 9.35 and B=100% ACN. The linear gradient elution from 90% B (0-1.5 minutes) to 50% B (8-11 minutes) and down to 45% B (12-15 minutes) was applied, followed by 9 minutes of column re-equilibration using the initial gradient conditions. The flow rate was 300 μL/minutes, column temperature 30° C. and sample injection volume 2 μl. ESI source conditions were set as follows: dry gas temperature 290° C. and flow 14 L/minutes, sheath gas temperature 350° C., nebulizer 45 psi, and flow 12 L/minutes, nozzle voltage 0 V, and capillary voltage −2000 V. Data were acquired in a Dynamic Multiple Reaction Monitoring (DMRM) mode with a total cycle time of 600 msec. Collision energies were optimized for each metabolite. LC-MS/MS data was processed using the Agilent Quantitative analysis software (version B.07.00, MassHunter Agilent technologies). Relative quantification of metabolites was based on EIC (Extracted Ion Chromatogram) areas for the monitored MRM transitions. For absolute quantification, calibration curves and the stable isotope-labeled internal standards (IS) were used and the concentrations of the compounds were calculated as the ratio of MS response (peak area) between the analyte and the IS, to account for matrix effects. Finally, the concentrations were reported to total protein content in brain tissue.

Mass spectrometry data further validated that the levels of NADH-producing enzymes, specifically the NAD-dependent isocitrate dehydrogenase (IDH) and the α-ketoglutarate hydroxylase, were upregulated. Furthermore, the NAD-IDH activity was increased 3-fold in Cyfip^{85.1}/+ brains in comparison to controls and the activity of α-KG-dependent hydroxylase was also significantly increased in Cyfip^{85.1}/+ brains (FIG. 4C). Moreover, the reduced form of nicotinamide adenine dinucleotide (NADH), was present at a significantly higher level (FIG. 4D). NADH levels were measured from brain lysates using the NAD/NADH Quantitation Colorimetric Kit (Biovision, K337). The enzyme activity of IDH was determined from isolated mitochondria using the BioVision kit (K756-100). Mitochondria were isolated as previously described (Depner (2014), Nat Protoc 9, 2796-2808; Yoon (2017), Neuron 93, 115-131).

Tracing of ¹³C-labelled glucose showed a drastic increase of isotope-labelled succinate in Cyfip^{85.1}/+ flies, while incorporation of the 13C-label into to malate and citrate/isocitrate did not change in the two genotypes (FIG. 4E). This confirms that the biochemical reactions between citrate/isocitrate and succinate, i.e., IDH and αKG, are overactive and shows that the TCA cycle overactivity is fed by glycolysis.

For isotope tracing, flies were fed with sucrose free food containing [U-13C]-glucose for 6 hours and immediately dissected in cold PBS 1×. For each sample, ten drosophila brains were pre-extracted and homogenized as described above. Brain extracts were analyzed by Hydrophilic Interaction Liquid Chromatography coupled to high resolution mass spectrometry (HILIC-HRMS) in negative ionization mode using a 6550 Quadrupole Time-of-Flight (Q-TOF) system interfaced with 1290 UHPLC system (Agilent Technologies) as previously described (Gallart-Ayala et al., 2018). Raw LCMS files were processed in Profinder B.08.00 software (Agilent Technologies) using the targeted data mining in isotopologue extraction mode. The metabolite identification was based on accurate mass and retention time matching against a database containing data on 600 polar metabolite standards (analyzed in the same analytical conditions). The Extracted Ion Chromatogram areas (EICs) of each isotopologue (M+0, M+1, M+2, M+3, . . . ) were corrected for natural isotope abundance (Midani (2017), Analytical biochemistry 520, 27-43) and the label incorporation or 13C enrichment was calculated based on relative isotopolgue abundance (in %), in each one of two analyzed conditions (Roci (2016), Analytical chemistry 88, 2707-2713).

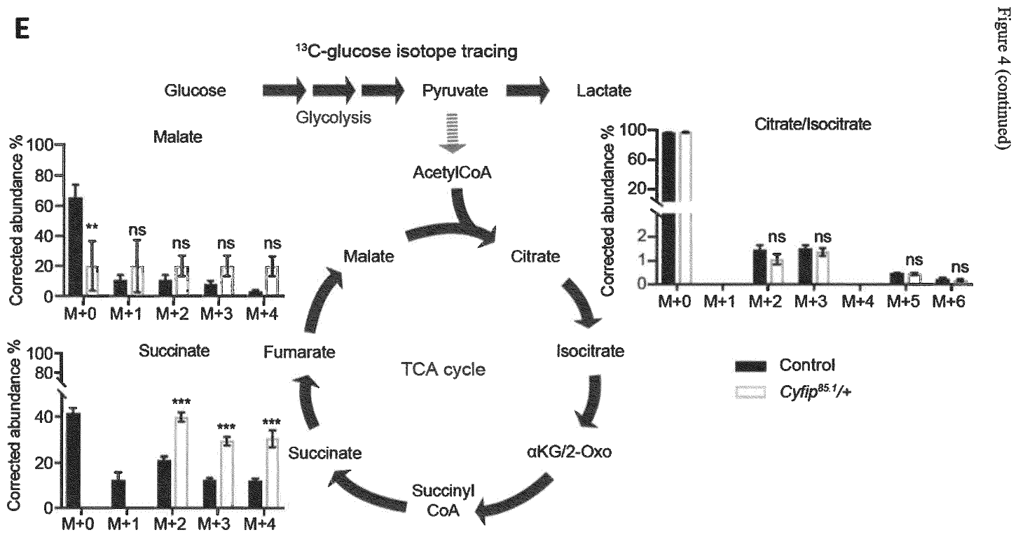

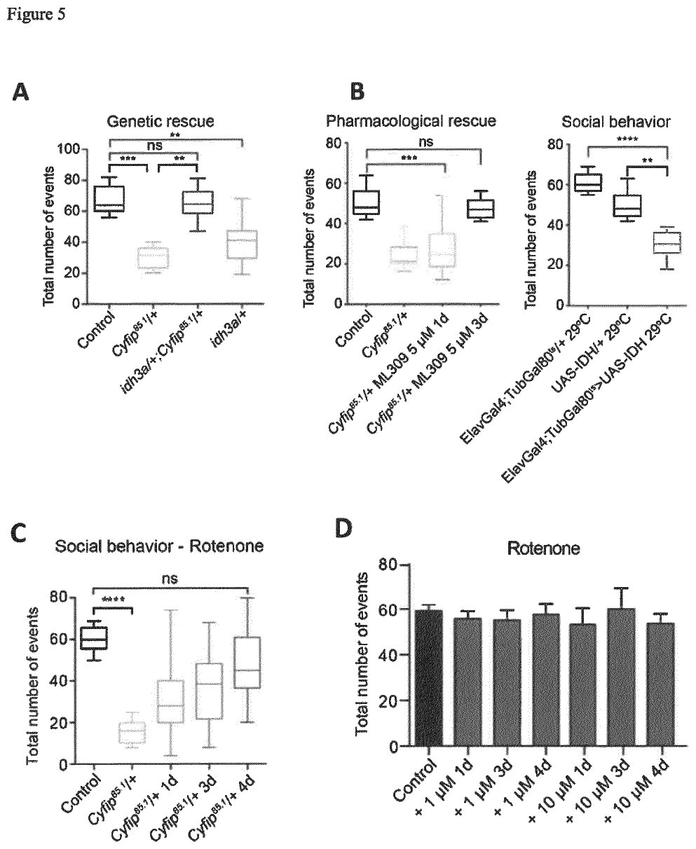

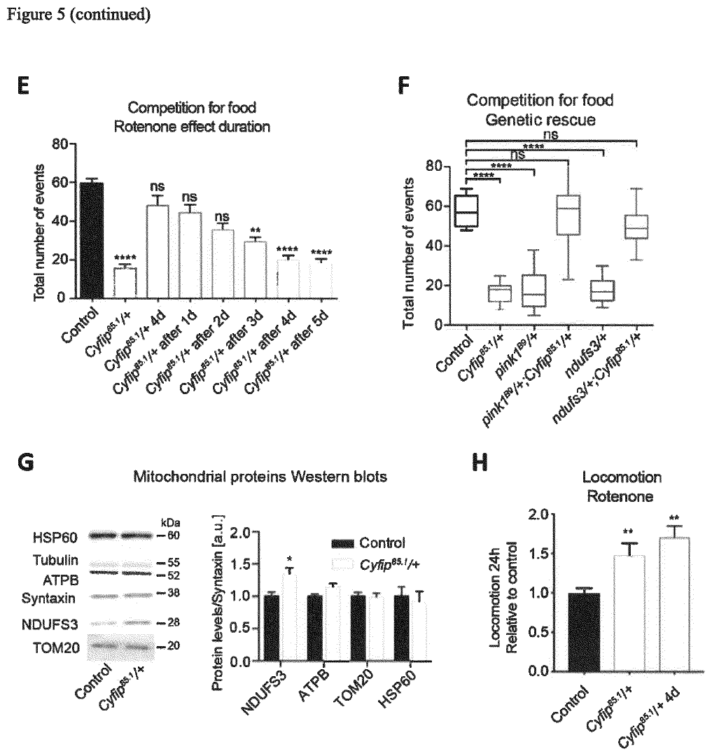

Example 5. Reducing mitochondrial activity or energy production rescues deficits in social behavior of Cyfip^{85.1}/+ flies. At first, the IDH activity was reduced to partially inhibit the TCA cycle and thereby normalize the energy production. Idh3a mutant flies (a gift from Hugo Bellen; Baylor College of Medicine, Houston), which have decreased IDH activity and reduced α-ketoglutarate levels compared to WT (Ugur (2017), Cell Rep 21, 3794-3806), had reduced social behavior in the food competition assay (see Example 2), but the double mutant idh3a/+;Cyfip^{85.1}/+ flies behaved similarly as the controls (FIG. 5A). Furthermore, pharmacological inhibition of IDH activity using ML309 (administered with the food at 50 mM concentration), a selective inhibitor of IDH that effectively lowers the production of α-ketoglutarate levels (Davis et al., 2014), also ameliorated the abnormal social feeding behavior in Cyfip^{85.1}/+ flies (FIG. 5B, left). In contrast, overexpression of IDH in the fly brain (FIG. 5B, right) showed a reduction in social behavior similar to the Cyfip^{85.1}/+ flies, suggesting that both too much and too little energy production leads to a social behavioral defect. These results further confirm the association of social behavior with the TCA cycle and highlight the importance of the IDH enzyme as a potential therapeutic target.

Next it was tested whether dampening mitochondrial activity and mitochondrial membrane potential might also normalize social behavior. Therefore, control and Cyfip^{85.1}/+ flies were fed with rotenone (an inhibitor of complex I) for 4 days at a concentration of approximately 500 times less than that used to model Parkinson disease in flies (1-10 μM in the food and administered as described in Example 8). Although this treatment had no effect on the behavior of wild type flies (FIG. 5D), it was found to ameliorate social deficits in Cyfip^{85.1}/+ mutants (FIG. 5C).

The effect of rotenone treatment was reversible (FIG. 5E). Of note, the same treatment did not restore the hyperactivity of Cyfip^{85.1}/+ mutants (FIG. 5H), suggesting that the mitochondrial phenotype is specific to social behavior. Moreover, reducing complex I activity by crossing Cyfip^{85.1}/+ flies to flies with a genetic reduction of complex I activity (Pink1^{B9}/+ mutants (Morais (2009), EMBO Mol Med 1, 99-111) also rescued social behavior to levels that were indistinguishable from controls (FIG. 5F). As for the Idh3a mutant, Pink1^{B9}/+ flies exhibited social deficits similar to those seen in Cyfip^{85.1}/+ mutants. Finally, western blotting analysis revealed that Cyfip^{85.1}/+ mutants harbored increased levels of the mitochondrial complex I subunit NDUFS3 (FIG. 5G), consistent with the mass spectrometry data. Assessments of ndufs3/+ mutant flies, harboring a mutation affecting mitochondrial complex I, also uncovered abnormal social behavior in these flies, whereas the double ndufs3/+;Cyfip^{85.1}/+ mutants were normal with regard to number of social interactions (FIG. 5F). In brief, for Western blot analysis, male fly heads were homogenized in 2× Laemmli buffer containing DTT. They were loaded on 10% acrylamide gels, transferred to PVDF membranes and probed with primary antibodies. The primary antibodies used: anti-Syntaxin (DSHB) at 1:3000, anti-ATP5B (Abcam) at 1:4000, anti-NDUFS3 (Abcam) at 1:4000, anti-HSP60 (Santa Cruz) at 1:4000, anti-Tom20 (Santa Cruz Biotech) at 1:1000, anti-Tubulin (DSHB) at 1:4000, and anti-Bruchpilot (DSHB) at 1:500. Detection was obtained using the Odyssey infrared imaging system (LI-COR, Bioscience). The fluorescence secondary antibodies DyLight 800 anti-rabbit and DyLight 800 anti-mouse were used at 1:5000 (Thermo Scientific).

Combined, these results suggest an unexpected, causal role for mitochondria and energy metabolism in the regulation of a complex social behavior.

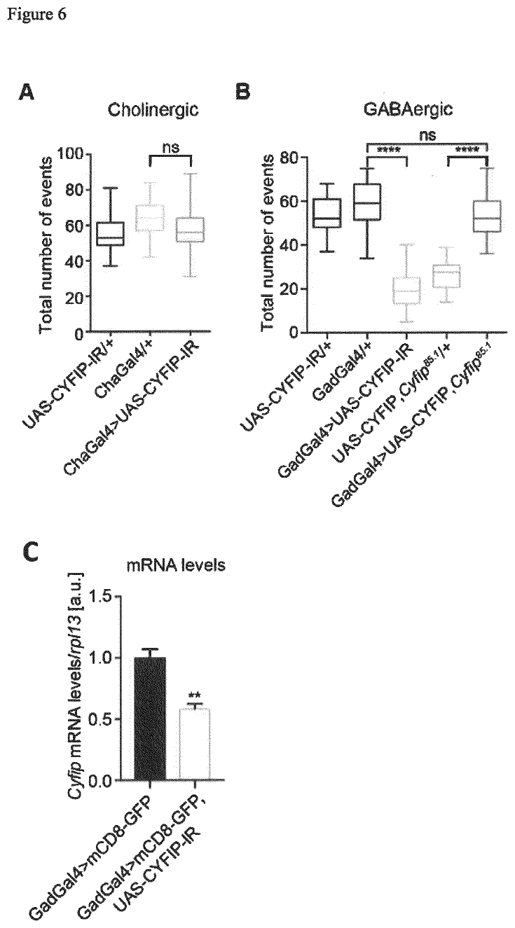

Example 6. The deficits in behavior of Cyfip^{85.1}/+ flies are mediated by GABAergic neurons. To identify whether the deficits in social behavior of Cyfip^{85.1}/+ flies were mediated by a specific cell type, Cyfip was knocked-down in either major excitatory (cholinergic) or inhibitory (GABAergic) neurons as described in Example 1. No effect on social behavior was observed when Cyfip levels were reduced in cholinergic neurons (FIG. 6A). However, depletion of Cyfip in GABAergic neurons recapitulated the feeding-dependent socialization defect seen in the Cyfip^{85.1}/+ flies (FIG. 6B), and this could be rescued by genetic reintroduction of Cyfip1. Attenuation of Cyfip mRNA levels specifically in GABAergic neurons was confirmed by gene expression analysis of FACS-sorted GABAergic neurons (FIG. 6C). mRNA levels were determined as described in Example 11. To isolate the GABAergic neuronal population from Drosophila brain, the enhancer-trap line Gad-GAL4 was used to drive the expression of GFP in GABAergic neurons of wild type and Cyfip^{85.1}/+ flies. Brains from adult flies (n=60) per genotype were dissected in cold 1×PBS. Tissues were dissociated using 0.05% trypsin-EDTA (Life Technologies) for 60 minutes at 37° C. in the dark. The GFP-positive cells were fractionated from the GFP negative population using a BD FACSAria I by BD Biosciences (San Jose) with a 70 μm nozzle tip at 60 psi. GFP was excited by a blue laser (488 nm) and detected using a 530/30 bandpass (BP) filter. Dead cells were excluded using propidium iodide (PI) at a final concentration of 1 μg/ml and detected using a 610/20 BP filter. Post sort purity check was performed to ensure clean fractionation of both the GFP-positive and -negative populations. Data were analyzed and presented with FlowJo software (Tree Star, Ashland, OR).

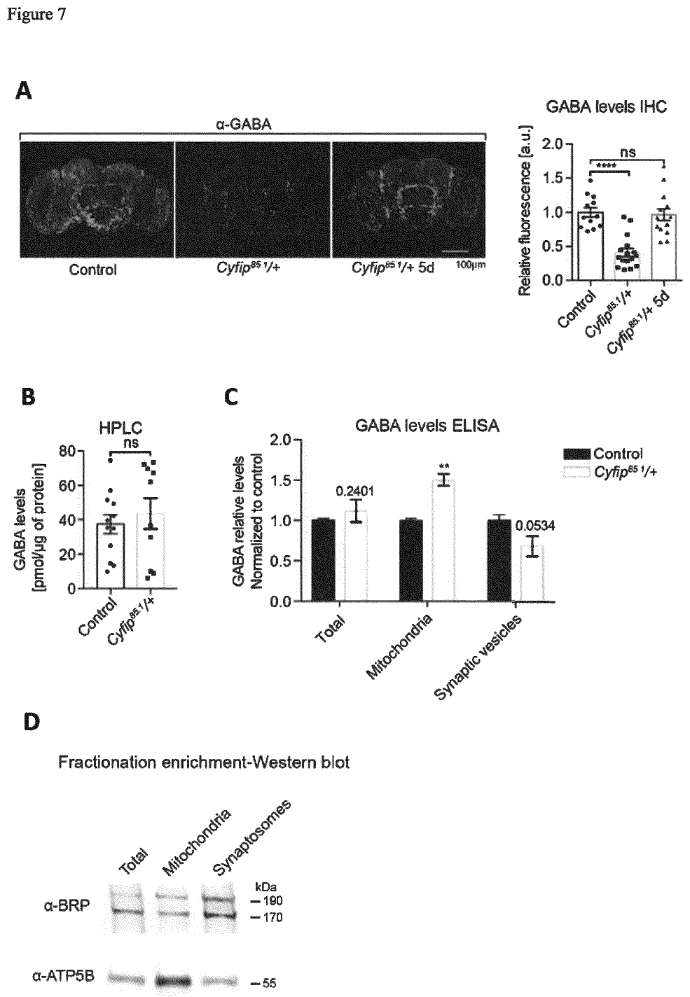

Example 7. GABA levels accumulate in the mitochondria of Cyfip^{85.1}/+ flies. Immunohistochemistry experiments of Cyfip^{85.1}/+ whole mount brains showed >50% reduced vesicular GABA levels (FIG. 7A). Briefly, fly brains were dissected under a stereomicroscope in phosphate-buffered saline 1× (PBS) for no longer than 1 hour and fixed in PBS 1× containing 3.7% formaldehyde for 10-15 minutes at room temperature using a rotator. After 3 washes of 10 minutes in PBT (PBS 1×, 0.3% Triton-X) the brains were blocked in 10% Normal Goat Serum in PBT for 30 minutes. The brains were then incubated with anti-GABA (Sigma) at 1:500 in 5% NGS in PBT at 4° C. overnight. After the primary antibody incubation, the brains were washed in PBT 3 times for 10 minutes per wash and incubated with secondaries Alexa Fluro 546 or 488 (Life Technologies, USA) at 1:1000 in 5% NGS in PBT for 1-2 hours at room temperature (RT). Then the brains were washed again in PBT for 3 times at RT for 10 minutes and mounted in Mowiol containing DAPI (1:1000) on a glass slide. Pictures were taken on the Olympus FluoView™ FV1000 Confocal Microscope and analyzed with ImageJ software.

However, HILIC-MS/MS revealed that the total concentration of GABA in Cyfip^{85.1}/+ mutants was not altered compared to controls as determined by (FIG. 7B). In brief, the samples for LC-MS were prepared as described in Example 4, but the quantification of GABA was performed using Hydrophilic Interaction Liquid Chromatography coupled to high resolution mass spectrometry (HILIC-HRMS) in positive ionization mode using a QExactive™ Hybrid Quadrupole-Orbitrap interfaced with Thermo Accela 1250 UPLC pump and CTC PAL Analytics autosampler. Metabolites were separated using a BEH Amide, 1.7 m, 100 mm×2.1 mm I.D. column (Waters, Massachusetts, US). The mobile phase was composed of A=20 mM ammonium formate and 0.1% FA in water and B=0.1% FA in ACN. The linear gradient elution from 95% B (0-2 minutes) to 65% B (14 minutes) and down to 50% B (16-18 minutes) was applied, followed by 4 minutes of column re-equilibration in the initial gradient conditions. The flow rate was 400 μL/minutes and the sample injection volume was 10 μL. HESI source conditions were set as follow: sheath gas flow at 60, Aux gas flow rate at 20, Sweep gas flow rate at 0, spray voltage at +3 kV, capillary temperature at 300° C., s-lens RF level at 60 and aux gas heater temperature at 300° C. The instrument was set to acquire over the mass range 60-900, with the MS acquisition parameters set as follows: resolution at 70′000 FWHM, 1 microscan, 1e6 AGC and maximum inject time at 100 ms. GABA was quantified by using a standard calibration curves and isotopic labeled internal standards. LC-MS data was processed using TraceFinder Clinical Research (version 4.1, Thermo Fischer Scientific).

Finally, GABA levels were measured by ELISA (GABA ELISA Enzyme immunoassay (IBL, ID59301) following a fractionation to enrich for mitochondria or synaptic vesicles (Depner (2014), Nat Protoc 9, 2796-2808). In brief, total brain lysates, isolated mitochondria and vesicles were used for GABA derivatization. The samples were incubated with a polyclonal antibody against GABA-derivative, together with assay reagent containing the GABA-derivative (tracer). The concentration of antibody-bound tracer is inverse proportional to the GABA concentration in the sample. Peroxidase conjugate was added detect the tracer and tetramethylbenzidine (TMB) served as a peroxidase substrate. The absorbance was measured in a microplate photometer at 450 nm. Surprisingly, the GABA concentration in the mitochondrial fraction of Cyfip^{85.1}/+ mutants was markedly higher compared to controls, reduced in the synaptic vesicles, and, as also observed by HILIC-MS/MS, unchanged in the total fraction (FIG. 7C).

Taken together, these data suggest that a reduction of Cyfip1 causes a redistribution of GABA from a vesicular/synaptic to a mitochondrial compartment, which leads to alterations in the behavior. The purity and enrichment of the mitochondria preparation was confirmed by Western blot (as described in Example 5) with the mitochondrial marker ATP5B and the synaptic marker BRP (FIG. 7D).

Example 8. GABA augmentation fully normalizes the behavioral abnormalities of Cyfip^{85.1}/+ flies. GABA levels were augmented in Cyfip^{85.1}/+ flies in three distinct ways: flies were fed either GABA, DL-2,4-diaminobutyric acid (DABA) which is a GABA-transaminase inhibitor and/or a GABA reuptake inhibitor or the anticonvulsant valproic acid (VPA) which induces GABA release (Chateauvieux (2010), Journal of biomedicine & biotechnology 2010; Coghlan (2012), Neurosci Biobehav Rev 36, 2044-2055; Reynolds (2007), Current medicinal chemistry 14, 2799-2812). All compounds were purchased from Sigma-Aldrich and were used as described before (Kanellopoulos (2012), J Neurosci. 32(38):13111-24; Zwarts (2011), PNAS 108, 17070-17075). In brief, the drugs were dissolved as per the manufacturer's instructions and used at the following concentrations: GABA at 50 μM, DABA 100 μM, Valproic acid (VPA) at 1 mM and Diazepam at 50 μM. Compounds in solution were mixed with Formula 4-24® Instant Drosophila Medium (blue food) in water. Approximately 50-60 adult flies were placed in plastic vials containing 500 μl of each solution on top of an agar matrix and allowed to feed ad libitum before behaviour analysis. Vehicle treatment consisted of solvent added to blue food alone.

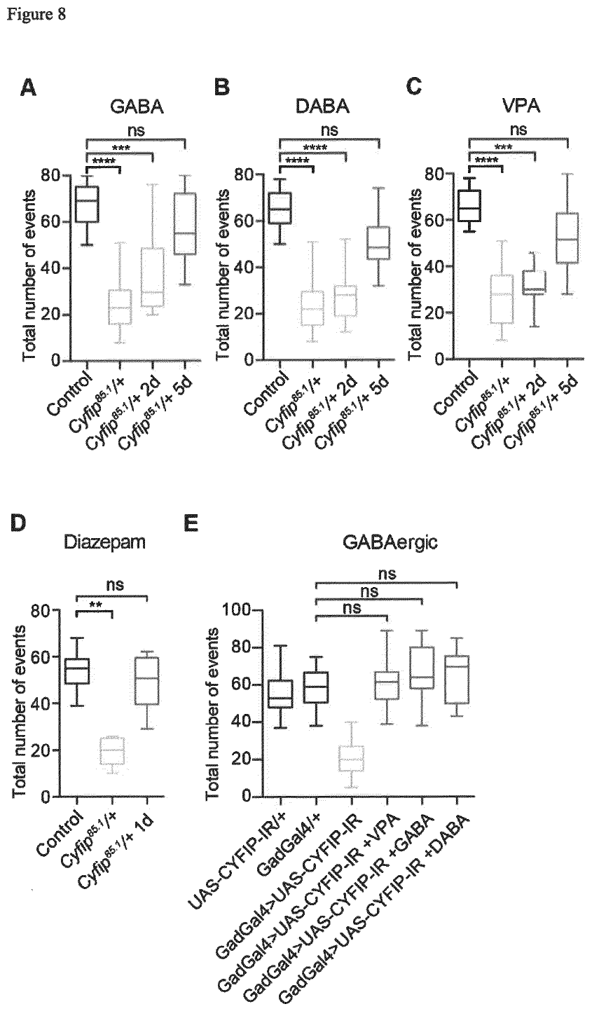

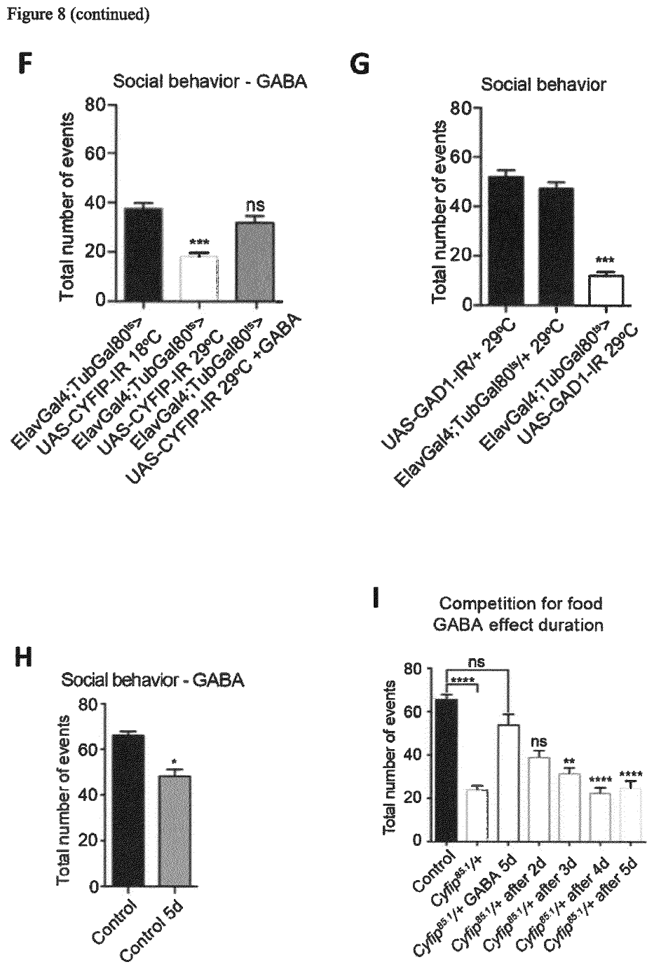

Feeding Cyfip^{85.1}/+ flies for 5 days with GABA, DABA or VPA ameliorated the social behavior deficit measured as described in Example 2 (FIG. 8A-C). Of note, the effect of GABA administration lasted 2 days post treatment (FIG. 8I). Interestingly, GABA feeding also rescued behavioral deficits in the adult pan-neuronal Cyfip^{85.1}/+ flies (FIG. 8F). Similarly, pan-neuronal abrogation in adult flies of the enzyme glutamic acid decarboxylase (GAD1) for 2 days led to a reduced number of social events (FIG. 8G). Furthermore, feeding flies diazepam (valium), a positive allosteric modulator of the GABA-A receptor, was also able to restore the social deficits of the Cyfip^{85.1}/+(FIG. 8D). These data show that augmentation of GABAergic signaling in adult is sufficient to ameliorate the behavioral deficits in flies resulting from reduced Cyfip dosage. Finally, underscoring the centrality of GABAergic signaling to this process, it was demonstrated that administration of GABA, DABA, or VPA to flies with GABAergic-specific Cyfip abrogation also restored the social interactions (FIG. 8E). Separate experiments in adult wild-type flies showed that a high GABA diet actually decreased social interaction (FIG. 8H), highlighting the importance of GABA homeostasis for normal behavior.

Example 9. Aralar is a GABA transporter which drives the excessive mitochondrial accumulation of GABA in Cyfip^{85.1}/+ flies. The experiments described in Example 6 to 8 suggested that a reduction of available GABA causes the behavioral deficits in Cyfip⁸⁵ 1 flies, and that the reduction in vesicular GABA is due to GABA being transported into mitochondria, where it might be catabolized into other intermediates. The inventors therefore set out to determine how GABA is transported into the mitochondria.

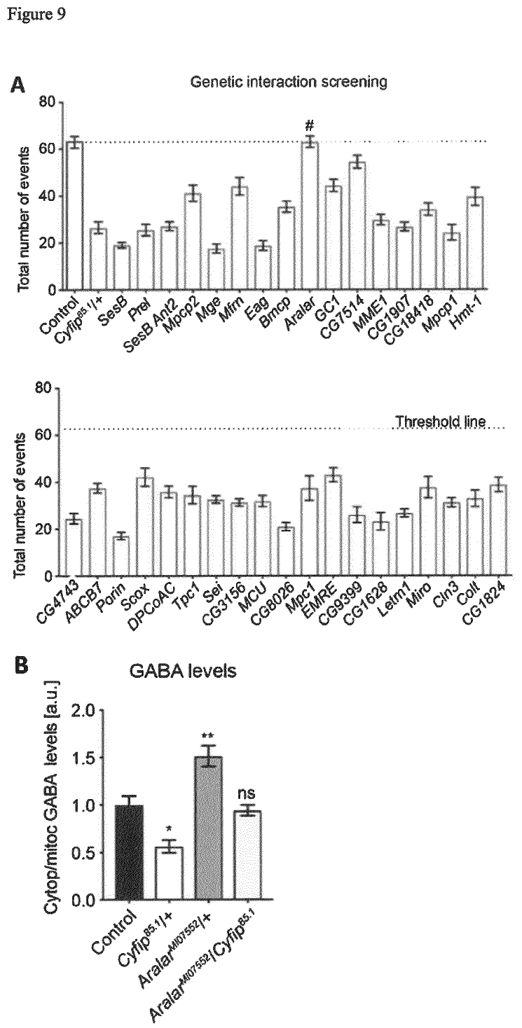

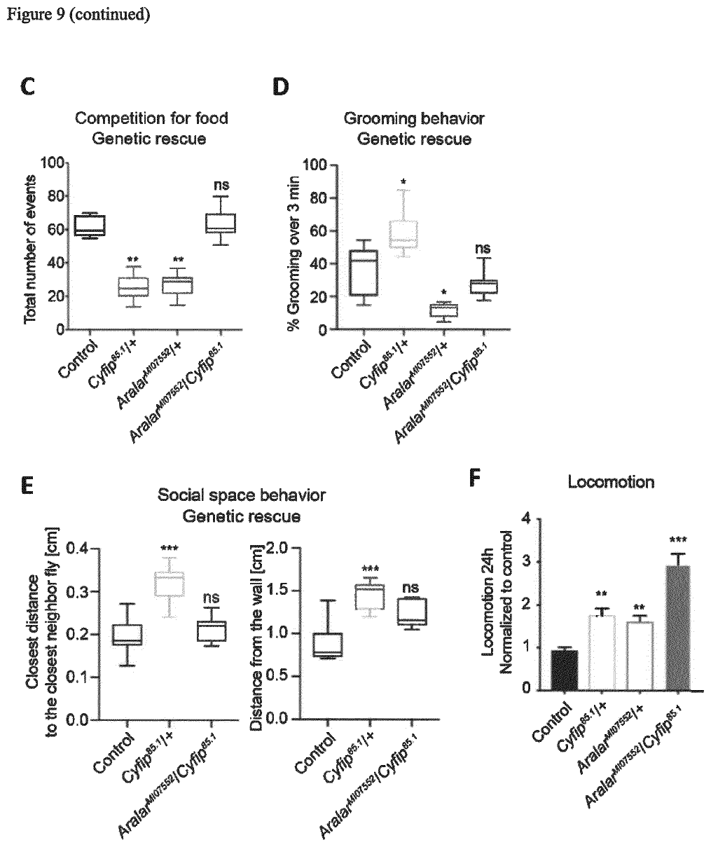

Using gene ontology analysis for GO:0005741 mitochondrial outer membrane based on the proteomics analysis described in Example 4, 36 candidate transporters in the fly were identified. Then, a genetic screen was performed to test their function in the Cyfip^{85.1}/+ flies. Flies with mutations in the 36 putative GABA transporters (purchased from the Bloomington stock center) were individually crossed with the Cyfip^{85.1}/+ flies and tested in the food competition social interaction assay described in Example 2. Of the 36 putative GABA transporters tested, only the Aralar^{MI07552} mutant showed a significant effect and completely ameliorated the behavioral deficits of Cyfip^{85.1}/+ flies (FIG. 9A).

To further assess whether GABA localization was affected by the mutation in Aralar, GABA levels were evaluated by ELISA upon fractionation of mitochondrial and cytosolic components comprising synaptic vesicles as described in Depner et al., 2014. Importantly, while cytosolic GABA levels were increased in Aralar^{MI07552}/+ mutant brains, the levels and localization of GABA across the cytosol and mitochondria in the Aralar^{MI07552}/Cyfip^{85.1} double mutant were indistinguishable from controls (FIG. 9B).

Moreover, the Aralar^{MI07552}/+ mutant flies exhibited a decreased number of social events similar as Cyfip^{85.1}/+ flies (see Example 3) in the food competition assay, but the social behavior of Aralar^{MI07552}/Cyfip^{85.1} double mutant flies was normal (FIG. 9C). This further suggests that homeostasis of mitochondrial GABA levels plays an important role in driving behavior and too much or too little GABA in the mitochondria, i.e. as compared to the cytosol, may lead to deficits in the behavior. Moreover, crossing the Cyfip^{85.1} fly with the Aralar^{MI07552} mutant ameliorated the Cyfip^{85.1}/+ grooming and social space behavior deficits (FIG. 7D, E), but not their hyperactivity (FIG. 7F), further confirming that the mitochondrial dysregulation and Aralar have a specific effect on behavior. Of note, the Aralar^{MI07552}/+ mutant flies further exhibited decreased grooming (FIG. 7D), and, similar to Cyfip^{85.1}/+ flies, hyperactivity (FIG. 7F).

Aralar has been so far described for its function as carrier exchanging a glutamate and a proton (H⁺) from the cytosol for an aspartate inside the mitochondria. The experiments described herein strongly suggest that Aralar is also a GABA transporter. Moreover, the data further suggest that Aralar is specifically the main mitochondrial GABA transporter which controls GABA signaling and thus behavior.

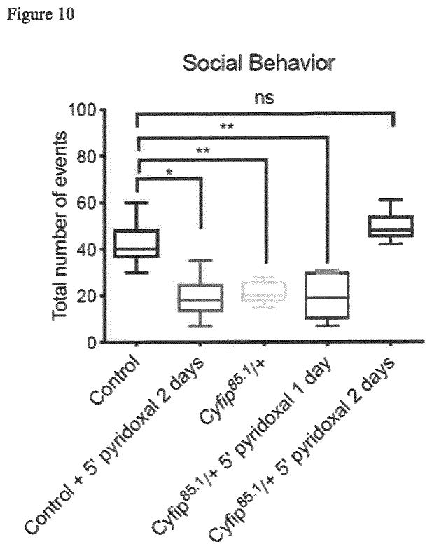

Example 10. Inhibition of Aralar fully normalizes the behavioral abnormalities of Cyfip^{85.1}/+ flies. Flies were fed for 1 or 2 days with fly food supplemented with the Aralar inhibitor pyridoxal 5′-phosphate hydrate (PLP; SIGMA-P9255; Palmieri (2001), EMBO J. 20(18):5060-9; Amoedo (2016), Biochim Biophys Acta 1863, 2394-2412) at a concentration of 1 mM in the food. Surprisingly, 2 days of pyridoxal 5′-phosphate hydrate administration reduced the number of social events (also see Example 2) of wild type flies to the level observed for in Cyfip^{85.1}/+ flies. However, in Cyfip^{85.1}/+ flies, the same administration of PLP increased the number of social events to the level observed for normal wild type flies (control) (FIG. 10). This further demonstrates that the activity of the GABA transporter Aralar regulates the behavior. The data further suggest that too high or too low GABA levels in the mitochondria, i.e. as compared to the cytosol, may lead to behavioral deficits. Therefore, a certain modulation of Aralar activity, for example with pyridoxal 5′-phosphate hydrate, may be used for the treatment of neurodevelopmental disorders. Moreover, the type of modulation, i.e. activation or inhibition may depend on the genotype of the subject, and/or other characteristics of the subject, i.e. the GABA levels and/or the mitochondrial activity and/or membrane potential.

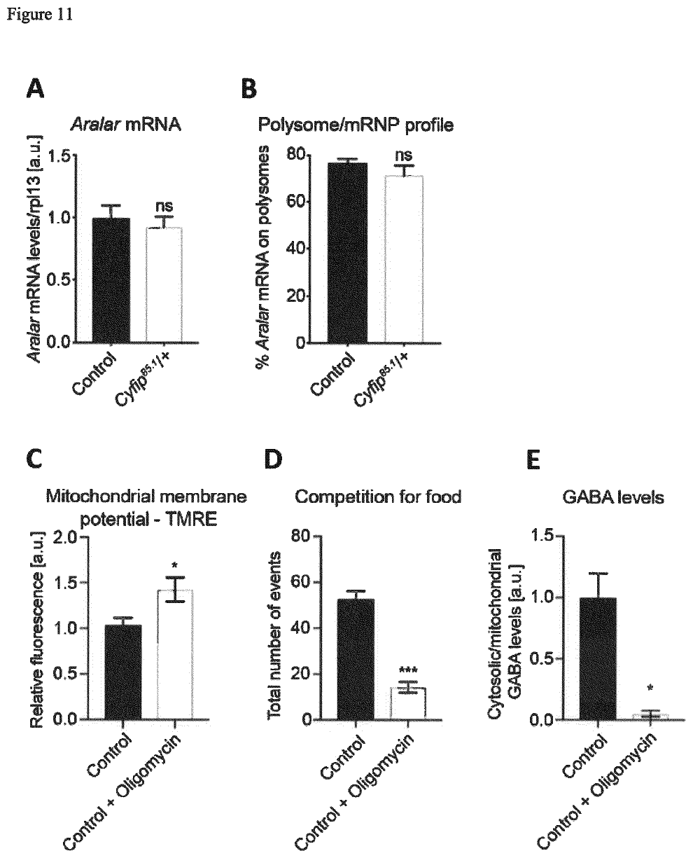

Example 11. Aralar activity is controlled by the proton gradient across the mitochondrial membrane rather than at the transcriptional or translational level. To address how a reduced dose of Cyfip could change the activity of Aralar, it was examined if Cyfip controls Aralar expression. Remarkably, neither Aralar mRNA level (FIG. 11A) nor its efficiency of translation/distribution of the mRNA on a polysomes/mRNP gradient (FIG. 11B) changed, suggesting that the regulation occurs at the level of Aralar activity.

In brief, the experiments were performed as following:

RNA Extraction, RT-PCR and quantitative PCR (qPCR): Total RNA, from 30 male fly heads 1 week old, was extracted using Trizol reagent (Invitrogen, Carlsbad, CA) according to manufacturer's instructions to extract total RNA. Before proceeding with the RT-PCR, RNA quantity and quality were determined with the NanoDrop 2000 U V Vis Spectrophotometer (Thermo, USA). Four independent samples for each genotype and 2 biological replicates have been collected. Total RNA was diluted to prepare aliquots of 200 ng/10 μl and used in RT-PCR reaction for 1 h at 37° C., using random primers, M-MLV enzyme (Invitrogen), buffer 5× M-MLV reaction buffer, RNAase out and dNTPs, according to manufacturer's instructions (Invitrogen). cDNA was diluted 1:20 and 5 μl cDNA was used for each qPCR 15 μl reaction. qPCR was conducted on a Light Cycler 96 (Roche, Switzerland) with SYBR Green PCR mix (Roche, Switzerland) with primers of the following genes: Cyfip, Aralar, gad, and ribosomal proteins L13 and L32 (encoding for the 60S ribosomal protein L13 and L32 respectively). All primer pairs were designed through the Fly Primer Bank (www.flyrnai.org/flyprimerbank) and were synthesized by IDT (Belgium) and Microsynth AG (Switzerland). Two technical replicates for each biological replicate were assessed. Statistical analysis was conducted using GraphPad Prism 6.0 software (La Jolla, CA). Data were evaluated by one-way analysis of variance (ANOVA) with Sidak's post hoc test and by Kruskal-Wallis's test with Dunn's correction or Mann-Whitney test.

Polysomes/mRNPs gradient: The protocol was slightly modified from (Napoli et al., 2008). Specifically, Drosophila heads were homogenized in lysis buffer (100 mM NaCl, 10 mM MgCl2, 10 mM Tris-HCL pH 7.5, 0.5 mM DTT, Protease inhibitor cocktail, 50 μg/mL CHX, RNAse OUT and RNAsin inhibitor 2.5 μl/mL). The lysates were incubated 5 min on ice and then centrifuged 5 min at 1000 g. In the supernatant Triton-X 20% and NaDoc 10% was added. Samples were then centrifuged 8 min at 12000 g at 4° C. and the supernatants centrifuged through 15%-50% (w/v) sucrose gradients for 2 hours at 37.000 rpm. Gradients were collected in 12 fractions. Equal volumes from each fraction was used for RNA extraction using Phenol-Chloroform-Isoammilic alcohol (according to manufacturer's conditions). 2 pg/μl of spike in Firefly Luciferase (FLuc) mRNA, (Promega) was added as loading control. The mRNA was used for RT and quantitative PCR as described above. Aralar mRNA level (Aralar primer For: TCCTGGGACTCTTTTCCGAAT (SEQ ID NO:6); Aralar primer Rev: GCCTGGAACTCCGAGAAGG (SEQ ID NO:7) was normalized to Fluc mRNA level (FLuc Primer For: ATCTGCCTCCTGGCTTCAAC (SEQ ID NO:8); FLuc Primer Rev: CGGTAGACCCAGAGCTGTTC (SEQ ID NO:9).

Finally, the proton gradient across the mitochondrial membrane was experimentally increased to study the effects on Aralar activity and social behavior in wild type flies. Flies were treated with a sublethal dose of oligomycin (500 μM in the food) to reduce Complex V activity (Spinazzi (2012), Nat Protoc 7, 1235-1246) reducing therefore the proton flux and increasing mitochondrial membrane potential, as confirmed by the TMRE staining (FIG. 11C). Notably, under these conditions, the competition for food of wild type flies was decreased (FIG. 11D) together with the level of available GABA in the cytosol (FIG. 11E), resembling the Cyfip^{85.1}/+ mutant condition. Since Aralar needs a proton for import of its substrate (i.e. GABA) into mitochondria, the equilibrium distribution of the transport substrates is set by the pH gradient across the mitochondrial membrane, which in turn is a function of fuel availability, oxidative phosphorylation and activity of the TCA cycle.

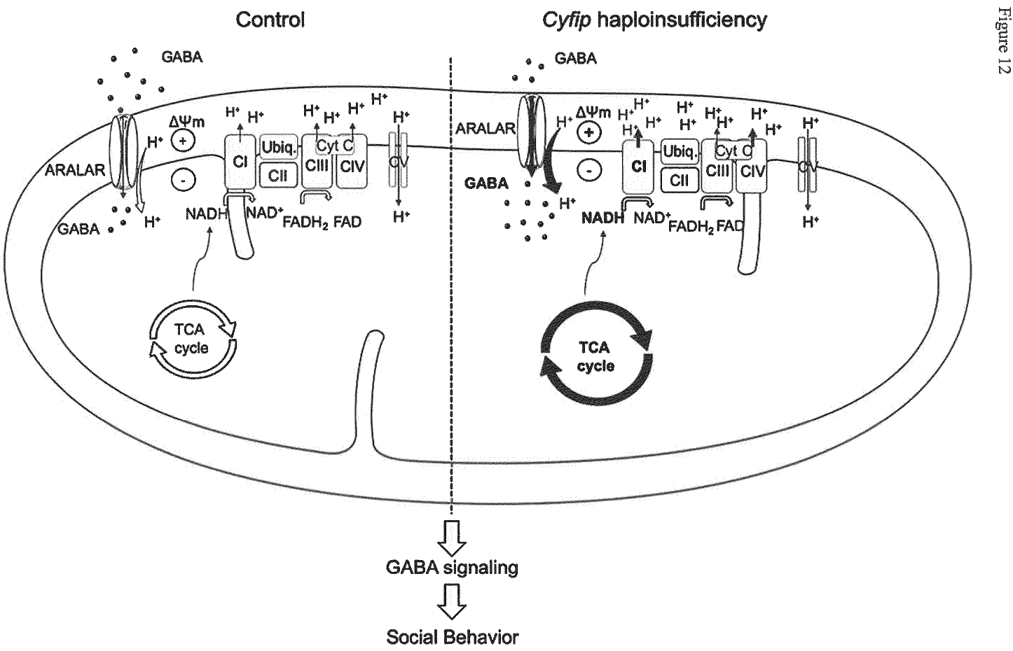

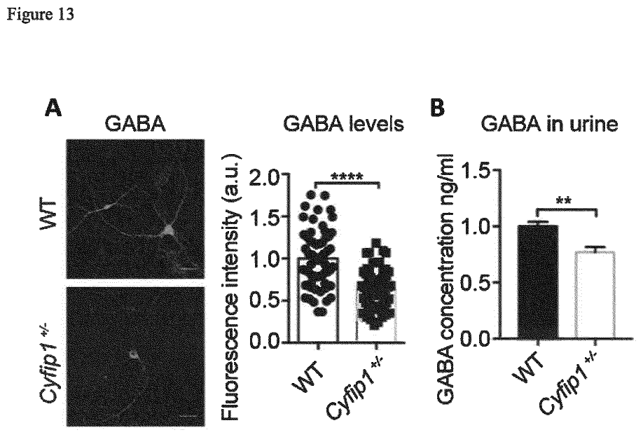

Example 12. GABA levels are reduced in neurons and the urine of Cyfip1+/− mice. As described in Example 7, the GABA levels in the brains of Cyfip^{85.1}/+ flies were reduced by 50% compared to control flies (FIG. 7). To parallel these studies in mammals, GABA levels were also analyzed in Cyfip1^{+/-} mice and were found to be reduced in primary neurons (FIG. 13). Furthermore, urine from WT and Cyfip1^{+/-} mice was analyzed by ELISA, and consistent with the data in flies, reduction of Cyfip1 levels in mammals led to decreased GABA levels (FIG. 13B).

Specifically, it was found that the GABA levels in the cytoplasm of primary neurons of Cyfip1+/− mice were reduced compared to the respective primary neurons of wild-type mice, as determined by immunofluorescence (FIG. 13A). It was further found that the GABA level in the urine of Cyfip1+/− mice was reduced compared to the urine of wild-type mice (FIG. 13B). This indicates that the findings in flies in the Examples described above may, at least in principle, be transferable to mammals and humans. Furthermore, this indicates that the GABA level in the urine of a patient may correlate with the GABA levels (i.e. outside of mitochondria) and/or the GABA signaling in the brain of said patient.

The experiments were performed as following:

Neuronal culture preparation and treatments: Mouse primary cortical neurons (E14-15) were prepared as previously described (De Rubeis (2013), Neuron 79, 1169-1182). Neurons were fixed and processed for immunofluorescence. At DIV14 neurons were fixed with 4% PFA/SEM (4% PFA, 0.12M sucrose, 3 mM EGTA, and 2 mM MgCl2 in PBS). After 1 hour in permeabilization/blocking solution (10% FBS and 0.2% Triton X-100 in PBS) neurons were incubated overnight with anti-GABA (1:100, Sigma). The following secondary antibodies were used: Alexa-488 anti-rabbit IgG (1:1000, Thermo Fisher Scientific).

Images acquisition and quantification: Z-stack images of 0.5 um were taken at a Zeiss LSM 780 GaAsP confocal microscope using a 63× oil immersion objective. Quantitative analysis was performed blind to the three different cultures. For each neuron, a total of 4 dendrites over a length of 25 μm from the cell body were analyzed. Quantification was performed manually using Image J software.

GABA measurement in mouse urine: Quantitative determination of GABA was performed using an ELISA immunoassay kit (IBL, International) following the manufacturer's protocol. Briefly, mouse spontaneous urine was collected for 4 hours. Then standards, controls and samples were placed into a specific plate. GABA was determined by ELISA using an anti-GABA antibody. The reaction was monitored at 450 nm and quantification of the samples was achieved by comparing the absorbance with a standard curve prepared with known samples provided in the kit.

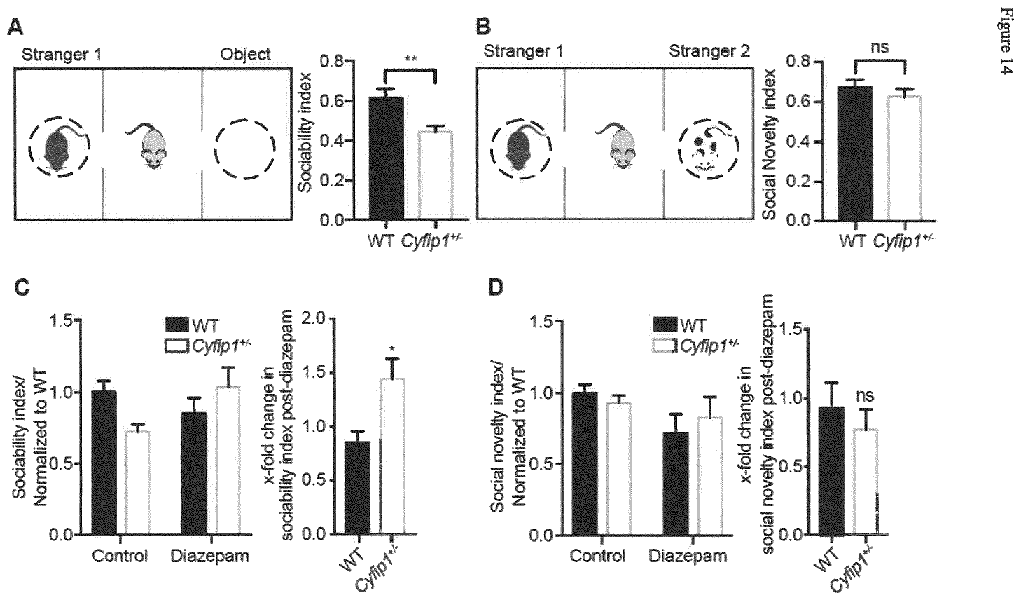

Example 13. Augmentation of GABA signaling restores deficits in social behavior of Cyfip1+/− mice. To determine the social behavior of WT mice and Cyfip1^{+/-} mice which have normal and reduced GABA levels in neurons, respectively, the mice were assessed in the three-chamber test, which allows investigating general sociability and interest in social novelty (Silverman (2015), Neuropsychopharmacology 40, 2228-2239; Yang (2012), The Journal of neuroscience 32, 6525-6541). Cyfip1+/− mice displayed a markedly lower social preference (FIG. 14A), suggesting impairment in socialization, but no difference in social novelty (FIG. 14B). As described in Example 8, treatment for 1 day with diazepam (valium), a positive allosteric modulator of the GABA-A receptor, restored the social deficits in Cyfip^{85.1}/+ flies. Thus, the WT and Cyfip1+/− mice were tested under both control and subsequent diazepam treatment in the three-chamber test. During the sociability phase, a significant genotype-by-treatment interaction was observed, which implied a differential effect of the treatment on the social preference in WT and Cyfip1+/− mice. Acute diazepam treatment rescued the social preference deficit in the Cyfip1+/− mice (FIG. 14C); in contrast, the same treatment caused a slight decrease in social preference in WT mice (FIG. 14C). When comparing within-subject the social preference index during control and diazepam-treated condition, Cyfip1+/− mice showed an approximately 50% increase in social preference after treatment (FIG. 14C). Diazepam treatment appeared to further slightly increase the preference for the novel mouse in Cyfip1+/− mice (and not in WT mice), but the differences between the two genotypes for the social novelty were not statistically significant (FIG. 14D).

The experiments were carried out as following:

### Sociability and Social Novelty Test

Sociability and social novelty were evaluated using the three-chamber test previously described (Crawley (2007), Brain Pathology 17(4), 448-459). Briefly, the set-up consisted of a rectangular non-transparent Plexiglas box divided into three compartments, separated by two partitions (each compartment: 30×19×21 cm). These partitions contained a small passage in the middle (8×7 cm; that can be sealed with small guillotine doors), which allowed the mouse to transition between adjacent compartments. During the acclimation phase, mice were allowed to explore the empty central compartment for 5 min. At the start of the test, both sliding doors were closed and each of the outer compartments were equipped with a wired cage (10 cm diameter, 20 cm high), one containing an object (50 ml Falcon Tube or a glue stick) and the other containing a mouse (i.e. stranger 1). Holding cages were used to avoid inter-male fighting but allow visual and olfactory contact between mice. During the sociability phase, the guillotine doors were removed and mice could freely explore the entire set-up for 10 min. It is expected from healthy mice that more interest is shown towards another mouse compared to an object. After 10 min, mice were placed into an empty housing cage, and the holding cage containing the object was replaced by a holding cage containing a novel mouse (i.e. stranger 2). During the next phase (social memory), the stranger 1 mouse has become familiar and it is expected that healthy mice show more interest towards the stranger 2 mouse compared to the stranger 1 mouse. The guillotine doors were removed and mice were allowed to explore the set-up for another 10 min. The location of the stranger 1 and stranger 2 mice was counterbalanced between animals and the box was cleaned with 70% EtOH solution after each animal. All stranger mice used were of the same sex as the tested mouse and had never been in contact with the test mice. Mice were recorded with a camera and were tracked with Ethovision software (Noldus, Wageningen, The Netherlands). Time spent in close proximity (<2 cm) to the holding cages was recorded. Preference index was calculated as: Time stranger 1/(Time stranger 1+Time object) and Timestranger 2/(Timestranger 1+Timestranger 2) for sociability and social memory/social novelty, respectively.

Treatment of Mice with Diazepam

Diazepam, a GABA-A positive modulator, was purchased from Roche (Basel, Switzerland) (diazepam already dissolved in saline at a concentration of 5 mg/ml). Diazepam was further diluted in saline and adjusted to the dose of 0.2 mg/ml. Mice were first tested in the three-chamber test in control conditions. 72 h afterwards, all mice were administered intraperitoneally with diazepam at 1% of bodyweight 30 min prior to re-testing. The stranger mice as well as the object were different from the initial test. All animals could function as their own within-subject control, thereby reducing the number of animals required in the experiment.

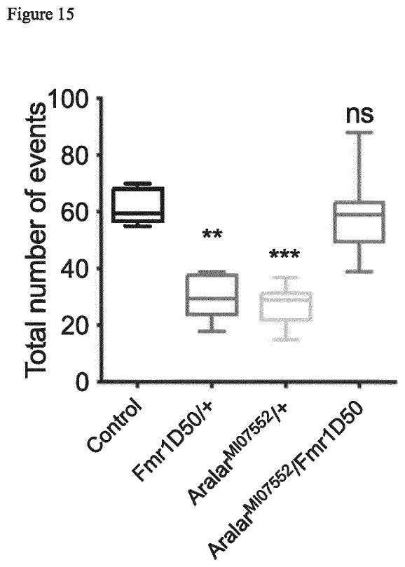

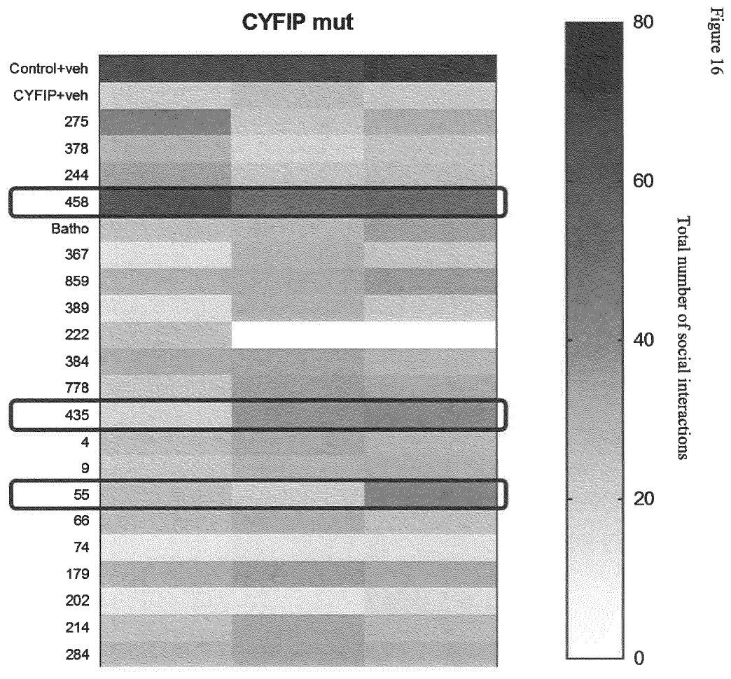

Example 14. Experiments with fmr1 mutant flies (null mutation) show that decreasing Aralar activity leads to a rescue of deficits in social behavior. Fmr1 mutant flies (Gatto (2014), Neurobiol Dis. 65:142-59) which are a well-known and largely used model for the Fragile X Syndrome, showed decreased social interactions, and an increased brain mitochondrial activity (FIG. 15), determined as for Cyfip^{85.1}/+ flies in the above Examples. Upon crossing fmr1 mutant flies to an Aralar mutant fly, the social deficits were rescued similarly to Example 9 for Aralar^{MI07552}/Cyfip^{85.1} flies. As fmr1 mutant flies have decreased GABA levels (Olmos-Serrano (2010), J Neurosci. 30(29):9929-38; Gatto (2014), Neurobiol Dis. 65:142-59; similarly to Cyfip^{85.1}/+ flies as shown in Example 7), this further suggest that Aralar might affect the behavior by regulating GABA signaling also in FXS. The Aralar-mediated mechanism may be therefore a general mechanism, and not limited to Cyfip haploinsufficiency. Furthermore, those data suggest that modulation of Aralar may be more promising for treating disorders that are associated with insufficient GABA signaling, i.e. FXS, than other approaches of modulating GABA signaling.

Example 15. Illustrative drug screening assays for identifying Aralar modulators and/or neuroactive drugs. The following assays/protocols may be used for identifying Aralar modulators, Aralar inhibitors, Aralar activators, or neuroactive drugs.

Compound Screening In Vivo Using Drosophila melanogaster

Candidate compounds, i.e. compounds that are related to energy metabolism may be selected from commercially available libraries. As first approach, 45 compounds from the Prestwick Chemical Library® may be already pre-selected. The compounds in solution may be added to Formula 4-24® Instant Drosophila Medium (blue food) in water and used at 50 μM final concentration. Male adult flies from control and Cyfip mutant flies may be placed in plastic vials containing the blue food and allowed to feed ad libitum for 24 hr. Vehicle treatment may consist of vehicle added to blue food alone. The next day, the flies may be tested in social behavior assays and a grooming assay and afterwards GABA levels may be measured in isolated mitochondria from flies that show behavioral improvement as described in Examples 2 and 3.

### Compound Screening In Vitro Using Proteoliposomes

Over the past years, suitable heterologous expression systems for membrane proteins have been used to improve the knowledge for both functional and structural properties of transporters. For the functional studies, proteoliposomes have been used as an experimental model to define function, kinetics and regulation of purified transporters (Scalise (2013), Pharmaceutics 5, 472-497). In particular, recombinant human Aralar may be embedded into the membrane bilayer (proteoliposomes; Palmieri (2001), EMBO J. 20(18):5060-9) by the freeze-thaw-sonication procedure. Inside the liposomes two important enzymes may be inserted, the GABA transaminase (GABA-T) that degrades the neurotransmitter GABA into succinic semialdehyde and the succinic semialdehyde dehydrogenase (SSADH) that catalyzes the chemical reaction succinic semialdehyde+NAD⁺+H₂O [symbol] succinate+NADH+2 H⁺. Both enzymes may be expressed and purified as soluble proteins (Scalise (2013), Pharmaceutics 5, 472-497). Briefly, mixtures containing liposomes, Aralar, the enzymes, necessary substrates (aspartate, alpha-ketoglutarate, NAD+) and buffer may be solubilized in non-ionic detergents freezing and then in slowly thawing these samples. After freezing, liposomes may be broken due to ice formation and, during the slow thawing, the proteins inserted into the phospholipid bilayer Scalise (2013), Pharmaceutics 5, 472-497). The reconstitution mixture may be passed through a column containing hydrophobic resin (Bio-Beads), for 10-20 times in order to remove the detergent with formation of liposomes containing the protein into the membrane bilayer (proteoliposomes). A very important outcome of the detergent removal method consists in the insertion of the proteins in unidirectional orientation, which in most cases, luckily corresponds to that of the transporter (Aralar) in the native membrane, i.e., the extracellular side of the transporter is outwardly oriented also in the artificial membrane (right-side-out orientation) (Scalise (2013), Pharmaceutics 5, 472-497). If necessary, excess protein that is not incorporated in the liposomes may be removed by centrifugation. These liposomes are an ideal test system for GABA transport: GABA outside the liposomes may be transported inside, where it is converted to succinate with the production of NADH, which levels may be measured by a commercially available kit (NADH Quantitation Colorimetric Kit, Biovision) using a microplate reader. The activity of any small-molecule drug on the transport may be quantified by adding the compound together with GABA to the liposomes, and comparing NADH production to vehicle control.

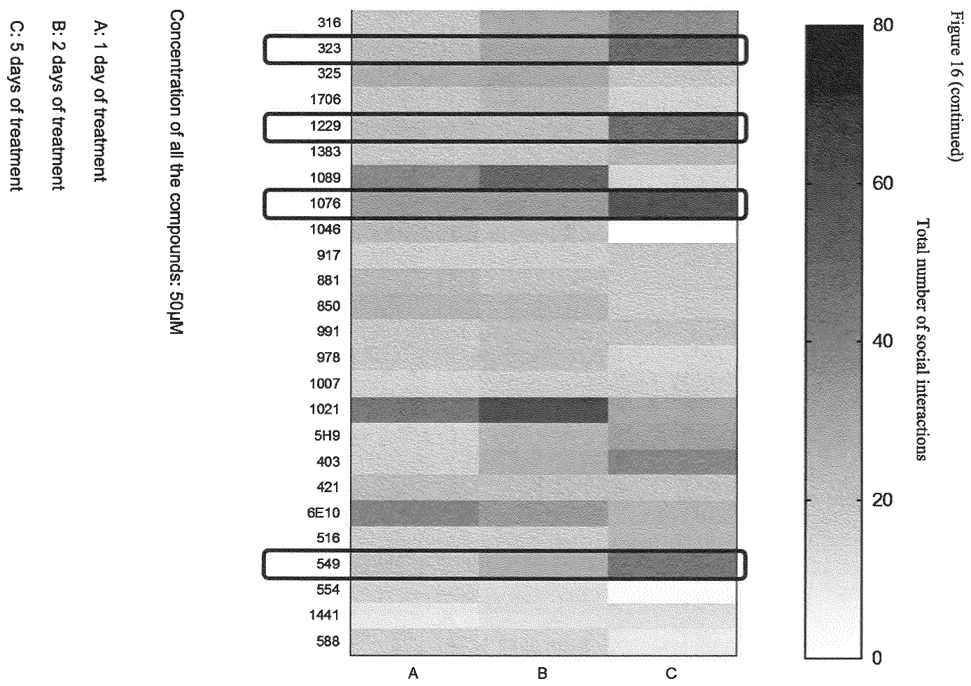

Example 16. Drug screening to identify compounds that ameliorate social behavior deficits in flies with Cyfip haploinsufficiency. Control (w1118) or CYFIP (Cyfip^{85.1}/+) flies were treated, i.e. fed, with various compounds from the Prestwick Chemical Library® as described in Examples 8 and 15. All compounds were used at a concentration of 50 μM. The total number of social interactions was analyzed in a competition for food assay at various time-points (1, 2 or 5 days) of treatment with the compound or vehicle controls as described in Example 2. It was found that among 46 compounds tested (Table 1), 7 showed a particular good effect (FIG. 16). These 7 hit compounds (Table 2) significantly increased the total number of social interaction of Cyfip haploinsufficient (Cyfip^{85.1}/+) flies at day 5 (two way anova analysis) and/or completely rescued the phenotype of Cyfip^{85.1}/+ flies.

The test compounds are shown in Table 1.

TABLE 1
Test compounds

| Prestw- | | |
|---|---|---|
| number | Chemical name | IUPAC name |
| Prestw-4 | Metformin | 1-carbamimidamido-N,N- |
| | hydrochloride | dimethylmethanimidamide |
| Prestw-9 | Hydrochlorothiazide | 6-Chloro-3,4-dihydro-2H-1,2,4-benzothiadiazine- |
| | | 7-sulfonamide 1,1-dioxide, 6-Chloro-7-sulfamyl- |
| | | 3,4-dihydro-1,2,4-benzothiadiazine 1,1-dioxide |
| Prestw-55 | Acetohexamide | 4-Acetyl-N- |
| | | [(cyclohexylamino)carbonyl]benzenesulfonamide |
| Prestw-66 | Minaprine | 4-methyl-N-(2-morpholin-4-ylethyl)-6- |
| | dihydrochloride | phenylpyridazin-3-amine dihydrochloride |
| Prestw-74 | Amitryptiline | 3-(10,11-Dihydro-5H-dibenzo[a,d]cyclohepten-5- |
| | hydrochloride | ylidene)-N,N-dimethyl-1-propanamine |
| | | hydrochloride |
| Prestw-179 | Phenformin | 1-(diaminomethylidene)-2-(2- |
| | hydrochloride | phenylethyl)guanidine hydrochloride |
| Prestw-202 | Tiratricol, 3,3′,5- | 2-[4-(4-hydroxy-3-iodophenoxy)-3,5- |
| | triiodothyroacetic acid | diiodophenyl]acetic acid |
| Prestw-214 | Gemfibrozil | 5-(2,5-dimethylphenoxy)-2,2-dimethylpentanoic |
| | | acid |
| Prestw-222 | Antimycin A | 3-methylbutanoate de (2R,6S,7R,8R)-3-[(3- |
| | | formamido-2-hydroxybenzoyl)amino]-8-hexyl- |
| | | 2,6-dimethyl-4,9-dioxo-1,5-dioxonan-7-yle |
| Prestw-244 | Glutethimide, para- | 3-(4-aminophenyl)-3-ethylpiperidine-2,6-dione |
| | amino | |
| Prestw-275 | Fenofibrate | propan-2-y1 2-[4-(4-chlorobenzoyl)phenoxy]-2- |
| | | methylpropanoate |
| Prestw-284 | Diethylcarbamazine | (N,N-Diethyl-4-methyl-1-piperazine carboxamide) |
| | citrate | Citrate Salt |
| Prestw-316 | Glibenclamide | 5-chloro-N-(4-[N- |
| | | (cyclohexylcarbamoyl)sulfamoyl]phenethyl)-2- |
| | | methoxybenzamide |
| Prestw-323 | Chlorpropamide | (1-[p-Chlorobenzenesulfony1]-3-propylurea) |
| Prestw-325 | Ascorbic acid | (5R)-5-[(1S)-1,2-dihydroxyethyl]-3,4-dihydroxy- |
| | | 2,5-dihydrofuran-2-one |
| Prestw-367 | Benfluorex | 2-[1-[3-(trifluoromethyl)phenyl]propan-2- |
| | | ylamino]ethyl benzoate |
| Prestw-378 | Bezafibrate | 2-[4-[2-[(4-chlorobenzoyl)amino]ethyl]phenoxy]- |
| | | 2-methylpropanoic acid |
| Prestw-384 | Probucol | 2,6-ditert-butyl-4-[2-(3,5-ditert-butyl-4- |
| | | hydroxyphenyl)sulfanylpropan-2- |
| | | ylsulfanyl]phenol |
| Prestw-389 | Ketoconazole | 1-[4-[4-[[(2R,4S)-2-(2,4-dichlorophenyl)-2- |
| | | (imidazol-1-ylmethyl)-1,3-dioxolan-4- |
| | | yl]methoxy]phenyl]piperazin-1-yl]ethanone |
| Prestw-398 | Oxantel pamoate | 3-[(E)-2-(1-methyl-5,6-dihydro-4H-pyrimidin-2- |
| (5H9) | | yl)ethenyl]phenol 4-[(3-carboxy-2- |
| | | hydroxynaphthalen-1-yl)methyl]-3- |
| | | hydroxynaphthalene-2-carboxylic acid |
| Prestw-403 | Thyroxine (L) | (2S)-2-amino-3-[4-(4-hydroxy-3,5- |
| | | diiodophenoxy)-3,5-diiodophenyl]propanoic acid |
| Prestw-421 | Suloctidil | 2-(octylamino)-1-(4-propan-2- |
| | | ylsulfanylphenyl)propan-1-ol |
| Prestw-435 | Cyclosporin A | cyclo[((2S)-2-aminobutyryl)-sarcosyl-N-methyl- |
| | | L-leucyl-L-valyl-N-methyl-L-leucyl-L-alanyl-D- |
| | | alanyl-N-methyl-L-leucyl-N-methyl-L-leucyl-N- |
| | | methyl-L-valyl-N-methyl-(4R)-4-[(E)-but-2-enyl]- |
| | | 4-methyl-L-threonyl] |
| Prestw-449 | Pilocarpine nitrate | (3S,4R)-3-ethyl-4-[(3-methylimidazol-4- |
| (6E10) | | yl)methyl]oxolan-2-one nitric acid |
| Prestw-458 | Melatonin | N-[2-(5-methoxy-1H-indol-3-yl)ethyl]acetamide |
| Prestw-516 | Lovastatin | [(1S,3R,7S,8S,8aR)-8-[2-[(2R,4R)-4-hydroxy-6- |
| | | oxooxan-2-yl]ethyl]-3,7-dimethyl-1,2,3,7,8,8a- |
| | | hexahydronaphthalen-1-yl] (2S)-2- |
| | | methylbutanoate |
| Prestw-549 | Trimetazidine | 1-[(2,3,4-trimethoxyphenyl)methyl]piperazine |
| | dihydrochloride | dihydrochloride |
| Prestw-554 | Tolazamide | 1-(azepan-1-yl)-3-(4-methylphenyl)sulfonylurea |
| Prestw-588 | Galanthamine | (4aS,6R,8aS)-5,6,9,10,11,12-hexahydro-3- |
| | hydrobromide | methoxy-11-methyl-4aH-[1]benzofuro[3a,3,2- |
| | | ef] [2] benzazepin-6-ol hydrobromide |
| Prestw-778 | Proadifen hydrochloride | 2-(diethylamino)ethyl 2,2-diphenylpentanoate |
| | | hydrochloride |
| Prestw-850 | Equilin | (9S,13S,14S)-3-hydroxy-13-methyl- |
| | | 9,11,12,14,15,16-hexahydro-6H- |
| | | cyclopenta[a]phenanthren-17-one |
| Prestw-859 | Fluvastatin sodium salt | (E,3R,5S)-7-[3-(4-fluorophenyl)-1-propan-2- |
| | | ylindol-2-y1]-3,5-dihydroxyhept-6-enoate sodium |
| Prestw-881 | Niacin | pyridine-3-carboxylic acid |
| Prestw-917 | Flurbiprofen | 2-(3-fluoro-4-phenylphenyl)propanoic acid |
| Prestw-978 | Memantine | 1-Amino-3,5,dimethyladamantane hydrochloride |
| | Hydrochloride | |
| Prestw-991 | Gliquidone | 1-cyclohexyl-3-[4-[2-(7-methoxy-4,4-dimethyl- |
| | | 1,3-dioxoisoquinolin-2- |
| | | yl)ethyl]phenyl]sulfonylurea |
| Prestw- | Fursultiamine | N-(4-Amino-2-methylpyrimidin-5-ylmethyl)-N- |
| 1007 | Hydrochloride | [4-hydroxy-1-methyl-2- |
| | | [(tetrahydrofurfuryl)dithio]-1-butenyl]formamide |
| | | monohydrochloride |
| Prestw- | Isradipine | 3-O-methyl 5-O-propan-2-yl 4-(2,1,3- |
| 1021 | | benzoxadiazol-4-yl)-2,6-dimethyl-1,4- |
| | | dihydropyridine-3,5-dicarboxylate |
| Prestw- | Repaglinide | 2-ethoxy-4-[2-[[(1S)-3-methyl-1-(2-piperidin-1- |
| 1046 | | ylphenyl)butyl]amino]-2-oxoethyl]benzoic acid |
| Prestw- | Ciprofibrate | 2-[4-(2,2-dichlorocyclopropyl)phenoxy]-2- |
| 1076 | | methylpropanoic acid |
| Prestw- | (S)-(−)-Cycloserine | (4S)-4-Amino-1,2-oxazolidin-3-one |
| 1089 | | |
| Prestw- | Aripiprazole | 7-[4-[4-(2,3-dichlorophenyl)piperazin-1- |
| 1229 | | yl]butoxy]-3,4-dihydro-1H-quinolin-2-one |
| Prestw- | Clofibrate | ethyl 2-(4-chlorophenoxy)-2-methylpropanoate |
| 1383 | | |
| Prestw- | Mevastatin | [(1S,7S,8S,8aR)-8-[2-[(2R,4R)-4-hydroxy-6- |
| 1441 | | oxooxan-2-yl]ethyl]-7-methyl-1,2,3,7,8,8a- |
| | | hexahydronaphthalen-1-yl] (2S)-2- |
| | | methylbutanoate |
| Prestw- | Donepezil | (RS)-2-[(1-benzyl-4-piperidyl)methyl]- |
| 1706 | hydrochloride | 5,6-dimethoxy-2,3-dihydroinden-1-one |
| Batho | Bathophenanthroline | 4,7-diphenyl-1,10-phenanthroline |

The hit compounds that strongly increased the number of social interactions in Cyfip haploinsufficient flies are shown in Table 2.

TABLE 2
Hit compounds

| Prestwick- | | CAS | |
|---|---|---|---|
| number | Chemical name | number | IUPAC name |
| Prestw-55 | Acetohexamide | 968-81-0 | 4-Acetyl-N-[(cyclohexylamino) |
| | | | carbonyl]benzenesulfonamide |
| Prestw-323 | Chlorpropamide | 94-20-2 | (1-[p-Chlorobenzenesulfonyl]- |
| | | | 3-propylurea) |
| Prestw-435 | Cyclosporin A | 59865-13-3 | |
| Prestw-458 | Melatonin | 73-31-4 | N-[2-(5-methoxy-1H-indol-3- |
| | | | yl)ethyl]acetamide |
| Prestw-549 | Trimetazidine | 13171-25-0 | 1-[(2,3,4-trimethoxyphenyl)methyl] |
| | dihydrochloride | | piperazine dihydrochloride |
| Prestw- | Ciprofibrate | 52214-84-3 | 2-[4-(2,2-dichlorocyclopropyl) |
| 1076 | | | phenoxy]-2-methylpropanoic acid |
| Prestw- | Aripiprazole | 129722-12-9 | 7-[4-[4-(2,3-dichlorophenyl) |
| 1229 | | | piperazin-1-yl]butoxy]-3,4-dihydro- |
| | | | 1H-quinolin-2-one |

1. A m method of treating a neurological disorder in a subject with Cyfip1 haploinsufficiency, which comprises administering to said subject a modulator of gamma-Aminobutyric acid (GABA) signaling.

2. The method according to claim 1, wherein said modulator is a modulator of Aralar.

3. The method according to claim 1, wherein said modulator increases GABA levels in the cytosol and/or synaptic vesicles and/or decreases GABA levels in the mitochondria of a cell containing GABA and Aralar.

4. The method according to claim 1, wherein said modulator indirectly modulates GABA signaling by modulating the mitochondrial respiratory capacity, the respiratory chain, and/or an enzyme of the TCA cycle.

5. The method according to claim 1, wherein said modulator modulates mitochondrial polarization and Aralar activity.

6. The method according to claim 1, wherein said modulator comprises GABA, a GABA analogue, a GABA receptor agonist, a positive allosteric modulator of a GABA receptor, a GABA reuptake inhibitor, and/or a GABA transaminase inhibitor, and/or wherein said modulator enhances GABA release.

7. The method according to claim 1, wherein said modulator comprises Diazepam, GABA, valproic acid, diaminobutyric acid, Acetohexamide, Chlorpropamide, Melatonin, Trimetazidine dihydrochloride, Ciprofibrate, and/or Aripiprazole.

8. The method according to claim 1, wherein said modulator comprises an inhibitor of isocitrate dehydrogenase (IDH) and/or an inhibitor of α-ketoglutarate hydroxylase.

9. (canceled)

10. The method according to claim 8, wherein said inhibitor is ML309 (2-(N-[2-(benzimidazol-1-yl)acetyl]-3-fluoroanilino)-N-cyclopentyl-2-(2-methylphenyl)acetamide).

11. The method according to claim 1, wherein said modulator is Acetohexamide or Cyclosporin A.

12. (canceled)

13. The method according to claim 1, wherein said subject has altered GABA levels in the brain and/or the urine.

14. The method according to claim 1, wherein said subject has an altered mitochondrial activity and/or membrane potential in a brain cell and/or neuron.

15. The method according to claim 1, wherein said subject is a human that comprises a copy number variation (CNV) at the 15q11.2 BP1-BP2 locus.

16. The method according to claim 1, wherein said neurological disorder is a neurodevelopmental disorder and/or psychiatric disorder.

17. The method according to claim 1, wherein said neurological disorder is a neurodevelopmental disorder and/or psychiatric disorder selected from the group consisting of autism spectrum disorder (ASD), schizophrenia, fragile X syndrome, obsessive-compulsive disorder, and disabilities featuring synaptic dysfunctions.

18. The method according to claim 17, wherein said neurodevelopmental disorder and/or psychiatric disorder is autism spectrum disorder (ASD).

19. The method according to claim 1, wherein said subject is a human, a domestic animal or a pet.

20. A method for preventing, alleviating, reversing, and/or stopping at least one behavioral deficit in a subject with Cyfip1 haploinsufficiency, which comprises administering to said subject a modulator of gamma-Aminobutyric acid (GABA) signaling, wherein said subject is not suffering from a neurological disorder, and wherein said at least one behavioral deficit is selected from the group consisting of: impaired learning, impaired memory such as impaired working memory, deficits in reading, writing and/or mathematics such as deficits in grammatical and/or mathematical reasoning, difficulties creating connections with other people, problems with friendships or romantic relationships, reduced social competence, lack of social awareness, unfocused thinking, reduced social engagement, lack of motivation, and reduced attention.

21. The method according to claim 20, wherein said modulator of GABA signaling is a modulator of Aralar.

22. The method according to claim 20, wherein said modulator of GABA signaling comprises Diazepam, GABA, valproic acid, diaminobutyric acid, Acetohexamide, Chlorpropamide, Melatonin, Trimetazidine dihydrochloride, Ciprofibrate, Aripiprazole, an inhibitor of isocitrate dehydrogenase (IDH) such as ML309, and/or an inhibitor of α-ketoglutarate hydroxylase.
